SEQUENCE LISTING (30 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - DNA, 1411 bp, Homo sapiens
ccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 3 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 4 - DNA, 2520 bp, Homo sapiens
ggatggttgtctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaa

SEQ ID NO: 5 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 6 - DNA, 2949 bp, Homo sapiens
agtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaaaaaaaaaaaaaaaaaa

SEQ ID NO: 7 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 8 - DNA, 2379 bp, Homo sapiens
gacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaaaaaaaaaaaaaaa

SEQ ID NO: 9 - protein, 234 aa, Homo sapiens
MLRVPEPRPGEAKAEGAAPPTPSKPLTSFLIQDILRDGAQRQGGRTSSQRQRDPEPEPEPEPEGGRSRAGAQNDQLSTGPRAAPEEAETLAETEPERHLGSYLLDSENTSGALPRLPQTPKQPQKRSRAAFSHTQVIELERKFSHQKYLSAPERAHLAKNLKLTETQVKIWFQNRRYKTKRKQLSSELGDLEKHSSLPALKEEAFSRASLVSVYNSYPYYPYLYCVGSWSPAFW

SEQ ID NO: 10 - DNA, 3281 bp, Homo sapiens
gcggtgcgggccgggcgggtgcattcaggccaaggcggggccgccgggatgctcagggttccggagccgcggcccggggaggcgaaagcggagggggccgcgccgccgaccccgtccaagccgctcacgtccttcctcatccaggacatcctgcgggacggcgcgcagcggcaaggcggccgcacgagcagccagagacagcgcgacccggagccggagccagagccagagccagagggaggacgcagccgcgccggggcgcagaacgaccagctgagcaccgggccccgcgccgcgccggaggaggccgagacgctggcagagaccgagccagaaaggcacttggggtcttatctgttggactctgaaaacacttcaggcgcccttccaaggcttccccaaacccctaagcagccgcagaagcgctcccgagctgccttctcccacactcaggtgatcgagttggagaggaagttcagccatcagaagtacctgtcggcccctgaacgggcccacctggccaagaacctcaagctcacggagacccaagtgaagatatggttccagaacagacgctataagactaagcgaaagcagctctcctcggagctgggagacttggagaagcactcctctttgccggccctgaaagaggaggccttctcccgggcctccctggtctccgtgtataacagctatccttactacccatacctgtactgcgtgggcagctggagcccagctttttggtaatgccagctcaggtgacaaccattatgatcaaaaactgccttccccagggtgtctctatgaaaagcacaaggggccaaggtcagggagcaagaggtgtgcacaccaaagctattggagatttgcgtggaaatctcagattcttcactggtgagacaatgaaacaacagagacagtgaaagttttaatacctaagtcattcctccagtgcatactgtaggtcattttttttgcttctggctacctgtttgaaggggagagagggaaaatcaagtggtattttccagcactttgtatgattttggatgagttgtacacccaaggattctgttctgcaactccatcctcctgtgtcactgaatatcaactctgaaagagcaaacctaacaggagaaaggacaaccaggatgaggatgtcaccaactgaattaaacttaagtccagaagcctcctgttggccttggaatatggccaaggctctctctgtccctgtaaaagagaggggcaaatagagagtctccaagagaacgccctcatgctcagcacatatttgcatgggagggggagatgggtgggaggagatgaaaatatcagcttttcttattcctttttattccttttaaaatggtatgccaacttaagtatttacagggtggcccaaatagaacaagatgcactcgctgtgattttaagacaagctgtataaacagaactccactgcaagagggggggccgggccaggagaatctccgcttgtccaagacaggggcctaaggagggtctccacactgctgctaggggctgttgcatttttttattagtagaaagtggaaaggcctcttctcaacttttttcccttgggctggagaatttagaatcagaagtttcctggagttttcaggctatcatatatactgtatcctgaaaggcaacataattcttccttccctccttttaaaattttgtgttcctttttgcagcaattactcactaaagggcttcattttagtccagatttttagtctggctgcacctaacttatgcctcgcttatttagcccgagatctggtctttttttttttttttttttttttttttccgtctccccaaagctttatctgtcttgactttttaaaaaagtttgggggcagattctgaattggctaaaagacatgcatttttaaaactagcaactcttatttctttcctttaaaaatacatagcattaaatcccaaatcctatttaaagacctgacagcttgagaaggtcactactgcatttataggaccttctggtggttctgctgttacgtttgaagtctgacaatccttgagaatctttgcatgcagaggaggtaagaggtattggattttcacagaggaagaacacagcgcagaatgaagggccaggcttactgagctgtccagtggagggctcatgggtgggacatggaaaagaaggcagcctaggccctggggagcccagtccactgagcaagcaagggactgagtgagccttttgcaggaaaaggctaagaaaaaggaaaaccattctaaaacacaacaagaaactgtccaaatgctttgggaactgtgtttattgcctataatgggtccccaaaatgggtaacctagacttcagagagaatgagcagagagcaaaggagaaatctggctgtccttccattttcattctgttatctcaggtgagctggtagaggggagacattagaaaaaaatgaaacaacaaaacaattactaatgaggtacgctgaggcctgggagtctcttgactccactacttaattccgtttagtgagaaacctttcaattttcttttattagaagggccagcttactgttggtggcaaaattgccaacataagttaatagaaagttggccaatttcaccccattttctgtggtttgggctccacattgcaatgttcaatgccacgtgctgctgacaccgaccggagtactagccagcacaaaaggcagggtagcctgaattgctttctgctctttacatttcttttaaaataagcatttagtgctcagtccctactgagtactctttctctcccctcctctgaatttaattctttcaacttgcaatttgcaaggattacacatttcactgtgatgtatattgtgttgcaaaaaaaaaaaaaaagtgtctttgtttaaaattacttggtttgtgaatccatcttgctttttccccattggaactagtcattaacccatctctgaactggtagaaaaacatctgaagagctagtctatcagcatctgacaggtgaattggatggttctcagaaccatttcacccagacagcctgtttctatcctgtttaataaattagtttgggttctctacatgcataacaaaccctgctccaatctgtcacataaaagtctgtgacttgaagtttagtcagcacccccaccaaactttatttttctatgtgttttttgcaacatatgagtgttttgaaaataaagtacccatgtctttattagattta

SEQ ID NO: 11 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 12 - DNA, 10661 bp, Homo sapiens
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttcaaaaaa

SEQ ID NO: 13 - protein, 472 aa, Homo sapiens
MLGTVKMEGHETSDWNSYYADTQEAYSSVPVSNMNSGLGSMNSMNTYMTMNTMTTSGNMTPASFNMSYANPGLGAGLSPGAVAGMPGGSAGAMNSMTAAGVTAMGTALSPSGMGAMGAQQAASMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 14 - DNA, 3396 bp, Homo sapiens
gggcttcctcttcgcccgggtggcgttgggcccgcgcgggcgctcgggtgactgcagctgctcagctcccctcccccgccccgcgccgcgcggccgcccgtcgcttcgcacagggctggatggttgtattgggcagggtggctccaggatgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctagctcccgggactggggggtttgtctggcatagccatgctggtagcaagagagaaaaaatcaacagcaaacaaaaccacacaaaccaaaccgtcaacagcataataaaatcccaacaactatttttatttcatttttcatgcacaacctttcccccagtgcaaaagactgttactttattattgtattcaaaattcattgtgtatattactacaaagacaaccccaaaccaatttttttcctgcgaagtttaatgatccacaagtgtatatatgaaattctcctccttccttgcccccctctctttcttccctctttcccctccagacattctagtttgtggagggttatttaaaaaaacaaaaaaggaagatggtcaagtttgtaaaatatttgtttgtgctttttccccctccttacctgaccccctacgagtttacaggtctgtggcaatactcttaaccataagaattgaaatggtgaagaaacaagtatacactagaggctcttaaaagtattgaaagacaatactgctgttatatagcaagacataaacagattataaacatcagagccatttgcttctcagtttacatttctgatacatgcagatagcagatgtctttaaatgaaatacatgtatattgtgtatggacttaattatgcacatgctcagatgtgtagacatcctccgtatatttacataacatatagaggtaatagataggtgatatacatgatacattctcaagagttgcttgaccgaaagttacaaggaccccaacccctttgtcctctctacccacagatggccctgggaatcaattcctcaggaattgccctcaagaactctgcttcttgctttgcagagtgccatggtcatgtcattctgaggtcacataacacataaaattagtttctatgagtgtataccatttaaagaatttttttttcagtaaaagggaatattacaatgttggaggagagataagttatagggagctggatttcaaaacgtggtccaagattcaaaaatcctattgatagtggccattttaatcattgccatcgtgtgcttgtttcatccagtgttatgcactttccacagttggacatggtgttagtatagccagacgggtttcattattatttctctttgctttctcaatgttaatttattgcatggtttattctttttctttacagctgaaattgctttaaatgatggttaaaattacaaattaaattgttaatttttatcaatgtgattgtaattaaaaatattttgatttaaataacaaaaataataccagattttaagccgtggaaaatgttcttgatcatttgcagttaaggactttaaataaatcaaatgttaacaaaagagcatttctgttattttttttcacttaactaaatccgaagtgaatatttctgaatacgatatttttcaaattctagaactgaatataaatgacaaaaatgaaaataaaattgttttgtctgttgttataatgaatgtgtagctagtaaaaaggagtgaaagaaattcaagtaaagtgtataagttgatttaatattccaagagttgagatttttaagattctttattcccagtgatgtttacttcatttttttttttttttttgacaccggcttaagccttctgtgtttcctttgagccttttcactacaaaatcaaatattaatttaactacctttcctccttccccaatgtatcacttttctttatctgagaattcttccaatgaaaataaaatatcagctgtggctgatagaattaagttgtgtccaaaaaaaaaaaaaaaaaa

SEQ ID NO: 15 - protein, 463 aa, Homo sapiens
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 16 - DNA, 2428 bp, Homo sapiens
cccgcccacttccaactaccgcctccggcctgcccagggagagagagggagtggagcccagggagagggagcgcgagagagggagggaggaggggacggtgctttggctgacttttttttaaaagagggtgggggtggggggtgattgctggtcgtttgttgtggctgttaaattttaaactgccatgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtcttacttaaaaaaaaaaaaaaattttgtgagtgactcggtgtaaaaccatgtagttttaacagaaccagagggttgtactattgtttaaaaacaggaaaaaaaataatgtaagggtctgttgtaaatgaccaagaaaaagaaaaaaaaagcattcccaatcttgacacggtgaaatccaggtctcgggtccgattaatttatggtttctgcgtgctttatttatggcttataaatgtgtattctggctgcaagggccagagttccacaaatctatattaaagtgttatacccggttttatcccttgaatcttttcttccagatttttcttttctttacttggcttacaaaatatacaggcttggaaattatttcaagaaggagggagggataccctgtctggttgcaggttgtattttattttggcccagggagtgttgctgttttcccaacattttattaataaaattttcagacataaaaaa

SEQ ID NO: 17 - protein, 457 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKKYRRDSASVVDQFFTDTEGLPYSINMNVFLPDITHLRTGLYKSQRPCVTHIKTEPVAIFSHQSETTAPPPAPTQALPEFTSIFSSHQTAAPEVNNIFIKQELPTPDLHLSVPTQQGHLYQLLNTPDLDMPSSTNQTAAMDTLNVSMSAAMAGLNTHTSAVPQTAVKQFQGMPPCTYTMPSQFLPQQATYFPPSPPSSEPGSPDRQAEMLQNLTPPPSYAATIASKLAIHNPNLPTTLPVNSQNIQPVRYNRRSNPDLEKRRIHYCDYPGCTKVYTKSSHLKAHLRTHTGEKPYKCTWEGCDWRFARSDELTRHYRKHTGAKPFQCGVCNRSFSRSDHLALHMKRHQN

SEQ ID NO: 18 - DNA, 3350 bp, Homo sapiens
tagtcgcggggcaggtacgtgcgctcgcggttctctcgcggaggtcggcggtggcgggagcgggctccggagagcctgagagcacggtggggcggggcgggagaaagtggccgcccggaggacgttggcgtttacgtgtggaagagcggaagagttttgcttttcgtgcgcgccttcgaaaactgcctgccgctgtctgaggagtccacccgaaacctcccctcctccgccggcagccccgcgctgagctcgccgacccaagccagcgtgggcgaggtgggaagtgcgcccgacccgcgcctggagctgcgcccccgagtgcccatggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaatgacagacctaactattcctgtgtaaaaacaacaaaaacaaacaaaagcaagaaaaccacaactaaaactggaaatgtatattttgtatatttgagaaaacagggaatacattgtattaataccaaagtgtttggtcattttaagaatctggaatgcttgctgtaatgtatatggctttactcaagcagatctcatctcatgacaggcagccacgtctcaacatgggtaaggggtgggggtggaggggagtgtgtgcagcgtttttacctaggcaccatcatttaatgtgacagtgttcagtaaacaaatcagttggcaggcaccagaagaagaatggattgtatgtcaagattttacttggcattgagtagtttttttcaatagtaggtaattccttagagatacagtatacctggcaattcacaaatagccattgaacaaatgtgtgggtttttaaaaattatatacatatatgagttgcctatatttgctattcaaaattttgtaaatatgcaaatcagctttataggtttattacaagttttttaggattcttttggggaagagtcataattcttttgaaaataaccatgaatacacttacagttaggatttgtggtaaggtacctctcaacattaccaaaatcatttctttagagggaaggaataatcattcaaatgaactttaaaaaagcaaatttcatgcactgattaaaataggattattttaaatacaaaaggcattttatatgaattataaactgaagagcttaaagatagttacaaaatacaaaagttcaacctcttacaataagctaaacgcaatgtcatttttaaaaagaaggacttagggtgtcgttttcacatatgacaatgttgcatttatgatgcagtttcaagtaccaaaacgttgaattgatgatgcagttttcatatatcgagatgttcgctcgtgcagtactgttggttaaatgacaatttatgtggattttgcatgtaatacacagtgagacacagtaattttatctaaattacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttattttgtggtaatatagtaagtttttttagaagacaattttcataacttgataaattatagttttgtttgttagaaaagttgctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaattttttgagagaatgagatgtttatatattaacgacaattttttttttggaaaataaaaagtgcctaaaaga

SEQ ID NO: 19 - protein, 477 aa, Homo sapiens
MTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 20 - DNA, 1892 bp, Homo sapiens
ggcgcccgcgcccgcccccgcgccgggcccggctcggcccgacccggctccgccgcgggcaggcggggcccagcgcactcggagcccgagcccgagccgcagccgccgcctggggcgcttgggtcggcctcgaggacaccggagaggggcgccacgccgccgtggccgcagatttgaaagaagccaacactaaaccacaaatatacaacaaggccattttctcaaacgagagtcagcctttaacgaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaattgctgatagta

SEQ ID NO: 21 - protein, 607 aa, Homo sapiens
MWMNSILPIFLFRSVRLLKNDPVNLQKFSYTSEDEAWKTYLENPLTAATKAMMRVNGDDDSVAALSFLYDYYMGPKEKRILSSSTGGRNDQGKRYYHGMEYETDLTPLESPTHLMKFLTENVSGTPEYPDLLKKNNLMSLEGALPTPGKAAPLPAGPSKLEAGSVDSYLLPTTDMYDNGSLNSLFESIHGVPPTQRWQPDSTFKDDPQESMLFPDILKTSPEPPCPEDYPSLKSDFEYTLGSPKAIHIKSGESPMAYLNKGQFYPVTLRTPAGGKGLALSSNKVKSVVMVVFDNEKVPVEQLRFWKHWHSRQPTAKQRVIDVADCKENFNTVEHIEEVAYNALSFVWNVNEEAKVFIGVNCLSTDFSSQKGVKGVPLNLQIDTYDCGLGTERLVHRAVCQIKIFCDKGAERKMRDDERKQFRRKVKCPDSSNSGVKGCLLSGFRGNETTYLRPETDLETPPVLFIPNVHFSSLQRSGGAAPSAGPSSSNRLPLKRTCSPFTEEFEPLPSKQAKEGDLQRVLLYVRRETEEVFDALMLKTPDLKGLRNAISEKYGFPEENIYKVYKKCKRGILVNMDNNIIQHYSNHVAFLLDMGELDGKIQIILKEL

SEQ ID NO: 22 - DNA, 2710 bp, Homo sapiens
aggagatgtgccaaactgttaagagtggttatttctgagcagaagaatgtggatgaattccattcttcctatttttcttttcaggtctgtgcggctgctaaagaacgacccagtcaacttgcagaaattctcttacactagtgaggatgaggcctggaagacgtacctagaaaacccgttgacagctgccacaaaggccatgatgagagtcaatggagatgatgacagtgttgcggccttgagcttcctctatgattactacatgggtcccaaggagaagcggatattgtcctccagcactgggggcaggaatgaccaaggaaagaggtactaccatggcatggaatatgagacggacctcactccccttgaaagccccacacacctcatgaaattcctgacagagaacgtgtctggaaccccagagtacccagatttgctcaagaagaataacctgatgagcttggagggggccttgcccacccctggcaaggcagctcccctccctgcaggccccagcaagctggaggccggctctgtggacagctacctgttacccaccactgatatgtatgataatggctccctcaactccttgtttgagagcattcatggggtgccgcccacacagcgctggcagccagacagcaccttcaaagatgacccacaggagtcgatgctcttcccagatatcctgaaaacctccccggaacccccatgtccagaggactaccccagcctcaaaagtgactttgaatacaccctgggctcccccaaagccatccacatcaagtcaggcgagtcacccatggcctacctcaacaaaggccagttctaccccgtcaccctgcggaccccagcaggtggcaaaggccttgccttgtcctccaacaaagtcaagagtgtggtgatggttgtcttcgacaatgagaaggtcccagtagagcagctgcgcttctggaagcactggcattcccggcaacccactgccaagcagcgggtcattgacgtggctgactgcaaagaaaacttcaacactgtggagcacattgaggaggtggcctataatgcactgtcctttgtgtggaacgtgaatgaagaggccaaggtgttcatcggcgtaaactgtctgagcacagacttttcctcacaaaagggggtgaagggtgtccccctgaacctgcagattgacacctatgactgtggcttgggcactgagcgcctggtacaccgtgctgtctgccagatcaagatcttctgtgacaagggagctgagaggaagatgcgcgatgacgagcggaagcagttccggaggaaggtcaagtgccctgactccagcaacagtggcgtcaagggctgcctgctgtcgggcttcaggggcaatgagacgacctaccttcggccagagactgacctggagacgccacccgtgctgttcatccccaatgtgcacttctccagcctgcagcgctctggaggggcagccccctcggcaggacccagcagctccaacaggctgcctctgaagcgtacctgctcgcccttcactgaggagtttgagcctctgccctccaagcaggccaaggaaggcgaccttcagagagttctgctgtatgtgcggagggagactgaggaggtgtttgacgcgctcatgttgaagaccccagacctgaaggggctgaggaatgcgatctctgagaagtatgggttccctgaagagaacatttacaaagtctacaagaaatgcaagcgaggaatcttagtcaacatggacaacaacatcattcagcattacagcaaccacgtcgccttcctgctggacatgggggagctggacggcaaaattcagatcatccttaaggagctgtaaggcctctcgagcatccaaaccctcacgacctgcaaggggccagcagggacgtggccccacgccacacacaacctctccacatgcctcagcgctgttacttgaatgccttccctgagggaagaggcccttgagtcacagacccacagacgtcagggccagggagagacctagggggtcccctggcctggatccccatggtatgcttgaatctgctccctgaacttcctgccagtgcctccccgtaccccaaaacaatgtcaccatggttaccacctacccagaagactgttccctcctcccaagacccttgtctgcagtggtgctcctgcaggctgcccgttaagatggtggcggcacacgctccctcccgcagcaccacgccagctggtgcggcccccactctctgtcttccttcaacttcagacaaaggatttctcaacctttggtcagttaacttgaaaactcttgattttcagtgcaaatgacttttaaaagacactatattggagtctctttctcagacttcctcagcgcaggatgtaaatagcactaacgatcgactggaacaaagtgaccgctgtgtaaaactactgccttgccactcactgttgtatacatttcttatttacgattttcatttgttatatatatatataaatatactgtatatatatgcaacattttatatttttcatggatatgtttttatcatttcaaaaaatgtgtatttcacatttcttggactttttttagctgttattcagtgatgcattttgtatactcacgtggtatttagtaataaaaatctatctatgtattacgtcacattaaaaaaaaaaa

SEQ ID NO: 23 - protein, 371 aa, Homo sapiens
MAATCEISNIFSNYFSAMYSSEDSTLASVPPAATFGADDLVLTLSNPQMSLEGTEKASWLGEQPQFWSKTQVLDWISYQVEKNKYDASAIDFSRCDMDGATLCNCALEELRLVFGPLGDQLHAQLRDLTSSSSDELSWIIELLEKDGMAFQEALDPGPFDQGSPFAQELLDDGQQASPYHPGSCGAGAPSPGSSDVSTAGTGASRSSHSSDSGGSDVDLDPTDGKLFPSDGFRDCKKGDPKHGKRKRGRPRKLSKEYWDCLEGKKSKHAPRGTHLWEFIRDILIHPELNEGLMKWENRHEGVFKFLRSEAVAQLWGQKKKNSNMTYEKLSRAMRYYYKREILERVDGRRLVYKFGKNSSGWKEEEVLQSRN

SEQ ID NO: 24 - DNA, 3149 bp, Homo sapiens
ctgagctcagggaggagctccctccaggctctatttagagccgggtaggggagcgcagcggccagatacctcagcgctacctggcggaactggatttctctcccgcctgccggcctgcctgccacagccggactccgccactccggtagcctcatggctgcaacctgtgagattagcaacatttttagcaactacttcagtgcgatgtacagctcggaggactccaccctggcctctgttccccctgctgccacctttggggccgatgacttggtactgaccctgagcaacccccagatgtcattggagggtacagagaaggccagctggttgggggaacagccccagttctggtcgaagacgcaggttctggactggatcagctaccaagtggagaagaacaagtacgacgcaagcgccattgacttctcacgatgtgacatggatggcgccaccctctgcaattgtgcccttgaggagctgcgtctggtctttgggcctctgggggaccaactccatgcccagctgcgagacctcacttccagctcttctgatgagctcagttggatcattgagctgctggagaaggatggcatggccttccaggaggccctagacccagggccctttgaccagggcagcccctttgcccaggagctgctggacgacggtcagcaagccagcccctaccaccccggcagctgtggcgcaggagccccctcccctggcagctctgacgtctccaccgcagggactggtgcttctcggagctcccactcctcagactccggtggaagtgacgtggacctggatcccactgatggcaagctcttccccagcgatggttttcgtgactgcaagaagggggatcccaagcacgggaagcggaaacgaggccggccccgaaagctgagcaaagagtactgggactgtctcgagggcaagaagagcaagcacgcgcccagaggcacccacctgtgggagttcatccgggacatcctcatccacccggagctcaacgagggcctcatgaagtgggagaatcggcatgaaggcgtcttcaagttcctgcgctccgaggctgtggcccaactatggggccaaaagaaaaagaacagcaacatgacctacgagaagctgagccgggccatgaggtactactacaaacgggagatcctggaacgggtggatggccggcgactcgtctacaagtttggcaaaaactcaagcggctggaaggaggaagaggttctccagagtcggaactgagggttggaactatacccgggaccaaactcacggaccactcgaggcctgcaaaccttcctgggaggacaggcaggccagatggcccctccactggggaatgctcccagctgtgctgtggagagaagctgatgttttggtgtattgtcagccatcgtcctgggactcggagactatggcctcgcctccccaccctcctcttggaattacaagccctggggtttgaagctgactttatagctgcaagtgtatctccttttatctggtgcctcctcaaacccagtctcagacactaaatgcagacaacaccttcctcctgcagacacctggactgagccaaggaggcctggggaggccctaggggagcaccgtgatggagaggacagagcaggggctccagcaccttctttctggactggcgttcacctccctgctcagtgcttgggctccacgggcaggggtcagagcactccctaatttatgtgctatataaatatgtcagatgtacatagagatctattttttctaaaacattcccctccccactcctctcccacagagtgctggactgttccaggccctccagtgggctgatgctgggacccttaggatggggctcccagctcctttctcctgtgaatggaggcagagacctccaataaagtgccttctgggctttttctaacctttgtcttagctacctgtgtactgaaatttgggcctttggatcgaatatggtcaagaggttggaggggaggaaaatgaaggtctaccaggctgagggtgagggcaaaggctgacgaagaggggagttacagatttcctgtagcaggtgtgggcttacagacacatggactgggctgggaggcgagcaaaggaagcagctgagactgttggagaacgcttacaagacttcatgcaagcaaggacatgaactcagaacactgaggtcagaagcatcctgctgtcatgacaccgctcgagtgaccttgaccttgaccaagtctgtcctgtttaggactgatttttcctattaggctagggtttggacctgatgttctcaagatgtctagaattgcatggctggccttgtggaatagatggttttgcattccagccaagtgtgctgtaaactgtatatctgtaatatgaatcccagcttttgagtctgacaaaatcagagttaggatcttgtaaaggaaaaaaaaaaaaaaacaaaacaaaatggagatgagtacttgctgagaaagaatgagggaaggagttggcatttgttgaaagtgtagtctttttctcttttttttttaattgcaacttttactttagatttaggaggtcgtgcgcaggtttgttacatgggtatattgtgtgatgctgagcttgggatgcgaatgatcctgtcacccaggtagtgagtatagcacccagtgaaactgtagtctcatgccaggcactgtgctagcccactctggctcatttaatcctctcctaagaagagaggagacacagcgtccccatttgacagatgcagaaagaggttccacaggtgtgccttgattctgtcctaaaaccgtttcccggaagcttttcctggtgtgggcgcttctaacctaatcctcaatcgattccagaactattactctgtttccacagtgatactgtgtctaggttttagggaggacagttcattgatgttacttaagaatgctttccaggtggaaagttccttaagtttgaggcttcaaattccatacagcacattaaaatcccattcatgagtttgaaatactgctctgttgtcttggaaataccaatcagattgttggctgaagtgatgtggataaagaagggatcttagaaaaactaaaaaaaaaaaaaaaaaa

SEQ ID NO: 25 - protein, 322 aa, Homo sapiens
MGLPERRGLVLLLSLAEILFKIMILEGGGVMNLNPGNNLLHQPPAWTDSYSTCNVSSGFFGGQWHEIHPQYWTKYQVWEWLQHLLDTNQLDANCIPFQEFDINGEHLCSMSLQEFTRAAGTAGQLLYSNLQHLKWNGQCSSDLFQSTHNVIVKTEQTEPSIMNTWKDENYLYDTNYGSTVDLLDSKTFCRAQISMTTTSHLPVAESPDMKKEQDPPAKCHTKKHNPRGTHLWEFIRDILLNPDKNPGLIKWEDRSEGVFRFLKSEAVAQLWGKKKNNSSMTYEKLSRAMRYYYKREILERVDGRRLVYKFGKNARGWRENEN

SEQ ID NO: 26 - DNA, 5467 bp, Homo sapiens
aacccactgctttattctgccctgagtggagattggttttggctcaggctgctttgtgaaactcagaagcattatcctctctgccaactccacgtcctagtcagagttttctgtgaaggcaagggcatggggttgccggagagaagaggattggtcctgcttttaagcctagctgaaattcttttcaagatcatgattctggaaggaggtggtgtaatgaatctcaaccccggcaacaacctccttcaccagccgccagcctggacagacagctactccacgtgcaatgtttccagtgggttttttggaggccagtggcatgaaattcatcctcagtactggaccaagtaccaggtgtgggagtggctccagcacctcctggacaccaaccagctggatgccaattgtatccctttccaagagttcgacatcaacggcgagcacctctgcagcatgagtttgcaggagttcacccgggcggcagggacggcggggcagctcctctacagcaacttgcagcatctgaagtggaacggccagtgcagtagtgacctgttccagtccacacacaatgtcattgtcaagactgaacaaactgagccttccatcatgaacacctggaaagacgagaactatttatatgacaccaactatggtagcacagtagatttgttggacagcaaaactttctgccgggctcagatctccatgacaaccaccagtcaccttcctgttgcagagtcacctgatatgaaaaaggagcaagacccccctgccaagtgccacaccaaaaagcacaacccgagagggactcacttatgggaattcatccgcgacatcctcttgaacccagacaagaacccaggattaataaaatgggaagaccgatctgagggcgtcttcaggttcttgaaatcagaggcagtggctcagctatggggtaaaaagaagaacaacagcagcatgacctatgaaaagctcagccgagctatgagatattactacaaaagagaaattctggagcgtgtggatggacgaagactggtatataaatttgggaagaatgcccgaggatggagagaaaatgaaaactgaagctgccaatactttggacacaaaccaaaacacacaccaaataatcagaaacaaagaactcctggacgtaaatatttcaaagactacttttctctgatatttatgtaccatgaggggaacaagaaactacttctaacgggaagaagaaacactacagtcgattaaaaaaattattttgttacttcgaagtatgtcctatatggggaaaaaacgtacacagttttctgtgaaatatgatgctgtatgtggttgtgattttttttcacctctattgtgaattctttttcactgcaagagtaacaggatttgtagccttgtgcttcttgctaagagaaagaaaaacaaaatcagagggcattaaatgttttgtatgtgacatgatttagaaaaaggtgatgcatcctcctcacataagcatccatatggcttcgtcaagggaggtgaacattgttgctgagttaaattccagggtctcagatggttaggacaaagtggatggatgccgggaagtttaacctgagccttaggatccaatgagtggagaatggggacttccaaaacccaaggttggctataatctctgcataaccacatgacttggaatgcttaaatcagcaagaagaataatggtggggtctttatactcattcaggaatggtttatctgatgccagggctgtcttcctttctcccctttggatggttggtgaaatactttaattgccctgtctgctcacttctagctatttaagagagaacccagcttggttcttttttgctccaagtgcttaaaaataagttggaaaaaggagacggtggtgtggaaatggctgaagagtttgctcttgtatccctatagtccaaggtttctcaatctgcacaattgacatttttggccggagtgttctttgtggtgagggctttcctgtgcattgtaagatgttcagcagtatccactcatggtctctaaccacttgacaccagaaaccccccagctgtgataacgcaaaatgtctctagacatcaccaaatgttccctgggggtggcaaatttgcccttgattgagaaccaccagtttagctagtcaatatgaggatggtggtttattctcagaagaaaaagatatgtaaggtcttttagctccttagagtgaagcaaaagcaagacttcaacctcaacctatctttatgttttaaatgttagggacaataagttgaaatagctagaggagcttcttttcagaaccccagatgagagccaatgtcagataaagtaagcatagtaatgtagcaggaactacaatagaagacattttcactggaattacaaagcagaattaaaattatattgtagaaggaaacaccaagaaaagaatttccagggaaaatcctctttgcaggtattaattcttataattttttgtcttttggattatctgtttactgtctcatctgaactgatcccaggtgaacggtttattgcctagatttgtactcagaggaattttttttgttttgttttgtcttttaagaaaggaaagaaaggatgaaaaaaataaacagaaaactcagctcaggcacaattgtcaccaaggagttaaaagcttcttcttcaatagaggaattgttctgggggtcctggagacttaccattgagccatgcaatctgggaagcacaggaataagtagacactttgaaaatggatttgaatgttctcatcccttttgcagcttttctttttggctctctcatgtccttggcttgctcctctattctacctctctttctccagcaataatatgcaaatgaagacatgtatccataagaaggagtgctcttcatcaactaatagagcacctaccacagtgtcatacctggtagaggtgagcaattcatattcaaaggttgcaaagtgtttgtaatatattcatgaggctggaagtaagaagaattaaaaatttgtcctaattacaatgagaaccattctaggtagtgatcttggagcacacatgaataactttctgaaggtgcaaccaaatccatttttatttctgcctggcttggtcacttctgtaaaggtttaacttagtgttgtcaagtaacagttactgaaagagctgagaaaaagaacaatgaacagcaacgatcttgactgtgcaactcagacattcctgcagaaaagacatatgttgctttacaagaaggccaaagaactatggggccttcccagcatttgactgttcattgcatagaatgaattaaatatccagttacttgaatgggtataacgcatgaatatttgtgtgtctgtgtgtgtgtctgagttgtgtgattttattaggggcatctgccaattctctcactgtggttccttctctgactttgcctgttcatcatctaaggaggctagatccttcgctgacttcaccattcctcaaacctgtaagtttctcacttcttccaaattggctttggctctttctgcaacctttccattcaagagcaatctttgctaaggagtaagtgaatgtgaagagtaccaactacaacaattctacagataattagtggattgtgttgtttgttgagagtgaaggtttcttggcatctggtgcctgattaaggcttgagtattaagttctcagcatatctctctattgtcttgacttgagtttgctgcattttctatgtgctgttcgtgacttggagaacttaaagtaatcgagctatgccaacttggggtggtaacagagtacttcccaccacagtgttgaaagggagagcaaagtcttatggataaaccctcctttcttttggggacacatggctctcacttgagaagctcacctgtgctgaatgtccacatggtcactaaacatgttatccttaaaccccccgtatgcctgagttgaaagggctctctcttattaggttttcatgggaacatgaggcagcaaatctattgctaagactttaccaggctcaaatcatctgaggctgatagatatttgacttggtaagacttaagtaaggctctggctcccaggggcataagcaacagtttcttgaatgtgccatctgagaagggagacccaggttgtgagttttcctttgaacacattggtcttttctcaaagttcctgccttgctagactgttagctctttgaggacagggactatgtcttatcaatcactattattttcctgttacctagcatgggacaagtacacaacacatatttgttcaatgaatgaatgaatgtcttctaaaagactcctctgattgggagaccatatctataattgggatgtgaatcatttcttcagtggaataagagcacaacggcacaaccttcaaggacatattatctactatgaacattttactgtgagactctttattttgccttctacttgcgctgaaatgaaaccaaaacaggccgttgggttccacaagtcaatatatgttggatgaggattctgttgccttattgggaactgtgagacttatctggtatgagaagccagtaataaacctttgacctgttttaaccaatgaagattatgaatatgttaatatgatgtaaattgctatttaagtgtaaagcagttctaagttttagtatttgggggattggtttttattatttttttcctttttgaaaaatactgagggatcttttgataaagttagtaatgcatgttagattttagttttgcaagcatgttgtttttcaaatatatcaagtatagaaaaaggtaaaacagttaagaaggaaggcaattatattattcttctgtagttaagcaaacacttgttgagtgcctgctatgtgcacggcatgggcccatatgtgtgaggagcttgtctaattatgtaggaagcaatagatctcggtagttacgtattgggcagatacttactgtatgaatgaaagaacatcacagtaatcacaatatcagagctgaattatcctcagtgtagcttcttggaattcagtttctggaactagagatagagcatttattaaaaaaaactcctgttgagactgtgtcttatgaacctctgaaacgtacaagccttcacaagtttaactaaattgggattaatctttctgtagttatctgcataattcttgtttttctttccatctggctcctgggttgacaatttgtggaaacaactctattgctactatttaaaaaaaatcagaaatctttccctttaagctatgttaaattcaaactattcctgctattcctgttttgtcaaagaattatatttttcaaaatatgtttatttgtttgatgggtcccaggaaacactaataaaaaccacagagaccagcctggaaaaaaaaaaaaaaaaaa

SEQ ID NO: 27 - DNA, 55 bp, Artificial Sequence
  Description of Artificial Sequence Synthetic oligonucleotide
tgtcccctccaccccacagtggggccactagggacaggattggtgacagacactt

SEQ ID NO: 28 - DNA, 120 bp, Artificial Sequence
  Description of Artificial Sequence Synthetic polynucleotide
ttcttcctccaattggtgacccccgttctcctgtggattcgggtcacctctcactcctttcatttgggcagctcccctaccccccttaccttctagtctggttctgggtacttttatctg

SEQ ID NO: 29 - DNA, 60 bp, Artificial Sequence
  Description of Artificial Sequence Synthetic oligonucleotide
gtacttttatctgtcccctccaccccacagtggggccactagggacaggattggtgacag

SEQ ID NO: 30 - DNA, 10 bp, Artificial Sequence
  Description of Artificial Sequence Synthetic oligonucleotide
ttggtgacag

(19) United States
(12) Patent Application Publication
(10) Pub. No.: US 2015/0023934 A1
(43) Pub. Date: Jan. 22, 2015

(54) GENERATION OF EPITHELIAL CELLS AND ORGAN TISSUE IN VIVO BY REPROGRAMMING AND USES THEREOF

(71) Applicant: THE TRUSTEES OF COLUMBIA UNIVERSITY IN THE CITY OF NEW YORK, New York, NY (US)

(72) Inventors: Flaminia IONAS, New York, NY (US); Michael M. SHEN, New York, NY (US)

(21) Appl. No.: 14/471,836

(22) Filed: Aug. 28, 2014

Related U.S. Application Data
(63) Continuation-in-part of application No. PCT/US2013/028265, filed on Feb. 28, 2013.
(60) Provisional application No. 61/604,455, filed on Feb. 28, 2012.

(51) Int. Cl. C12N 15/86; A61K 35/28; A61K 35/36

(52) U.S. Cl. CPC C12N 15/86; A61K 35/36; A61K 35/28; C12N 2770/00042
USPC 424/93.21; 435/456; 435/325

(57) ABSTRACT

The present invention encompasses methods for reprogramming fibroblast cells in culture, which are able to generate generic epithelial cells therefrom.

This application is a continuation-in-part of International Application No. PCT/US2013/028265, filed on Feb. 28, 2013, which claims priority to U.S. Application Ser. No. 61/604,455, filed on Feb. 28, 2012, the contents of each of which are hereby incorporated by reference in their entireties.

### GOVERNMENT SUPPORT

The invention was made with government support under Grant No. R01 DK076602 awarded by the National Institute of Diabetes and Digestive and Kidney Diseases, and under Grant No. P01 CA154293 awarded by the National Cancer Institute. The Government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Prostate disorders, such as prostatitis, benign prostate hyperplasia and prostate cancer are the most common male-related pathologies. Despite recent advances in basic and translational research, prostate cancer remains the second leading cause of cancer in men and a complete cure remains elusive. Complications in the clinic arise from prostate cancer phenotypic heterogeneity, imperfect early prognostic markers able to predict the evolution of the disease to aggressive forms, and the progression to castration-resistant forms.

### SUMMARY OF THE INVENTION

The present invention relates generally to the finding that induced pluripotent stem cells (iPSCs) can be directly differentiated and that mouse and human fibroblasts can be transdifferentiated into prostate and urinary bladder epithelium.

An aspect of the invention is directed to a method for reprogramming embryonic fibroblast cells in culture to epithelial cells. In one embodiment, the method comprises: (a) isolating embryonic fibroblasts (EFs); (b) infecting EFs with a retrovirus comprising a reprogramming factor; and (c) incubating for at least 24 hours at about 37° C. In another embodiment, the method further comprises switching culture medium to a serum-free basal epithelial medium. In some embodiments, the basal epithelial medium contains EGF, FGF, or a combination of the listed growth factors. In one embodiment, the embryonic fibroblasts (EF) has a wild-type genotype, an Oct4-GFP knock-in genotype, or a Nkx3.1-lacZ knock-in genotype. In one embodiment, the embryonic fibroblasts (EF) have a GATA6CreERT2; R26R-CAG-YFP genotype. In one embodiment, the embryonic fibroblasts (EF) have a CK18CreERT2; R26R-Tomato genotype. In another embodiment, the retrovirus is a Rebna retrovirus. In one embodiment, the embryonic fibroblasts are mouse embryonic fibroblasts. In a further embodiment, the reprogramming factor is Oct4, Sox2, Klf4, c-Myc, or a combination of the listed reprogramming factors. In some embodiments, the epithelial cells are induced epithelial cells. In yet other embodiments, the induced epithelial cells express cytokeratin 5 (CK5), CK8, CK14, CK18, beta-catenin, E-cadherin, or a combination of such listed markers. In one embodiment, the induced epithelial cells express EpCAM, CD24, or a combination thereof. In some embodiments, the induced epithelial cells are stably maintained for at least 3 passages, at least 4 passages, at least 5 passages, at least 6 passages, at least 7 passages, at least 8 passages, at least 9 passages, at least 10 passages, at least 11 passages, at least 12 passages, at least 13 passages, at least 14 passages, or at least 15 passages. In further embodiments, the induced epithelial cells are further differentiated in prostate epithelia or bladder epithelia. In some embodiments, the retrovirus is a lentivirus. In another embodiment, the lentivirus is doxycycline regulated.

In one embodiment, the embryonic fibroblasts of (a) express CD140. In another embodiment, the embryonic fibroblasts of (a) do not express CD11, EpCAM, CD24, or a combination thereof.

An aspect of the invention is directed to a method for reconstituting induced epithelial cells into an organ tissue. In one embodiment, the method comprises: (a) isolating induced epithelial cells prepared according to the method described above; (b) transducing the induced epithelial cells with a retrovirus comprising a master regulatory gene; (c) recombining the induced epithelial cells with mesenchymal cells; and (d) performing a graft in an immunodeficient subject. In another embodiment, the master regulatory gene is a master regulatory gene for prostate development. In a further embodiment, the master regulatory gene for prostate development comprises NKX3.1, Androgen Receptor (AR), FOXA1, FOXA2, or a combination of the listed master regulatory genes. In some embodiments, the master regulatory gene is a master regulatory gene for bladder development. In other embodiments, the master regulatory gene for bladder development comprises KLF5, Pparγ, Grhl3, Ovol1, Foxa1, Elf3, Ehf, or a combination of the listed master regulatory genes. In further embodiments, the mesenchymal cells comprise urogenital mesenchyme. In one embodiment, the graft is a renal graft. In another embodiment, the organ tissue is prostate epithelial tissue. In a further embodiment, the organ tissue is bladder epithelial tissue. In some embodiments, the organ tissue expresses p63 and CK5 in the basal layer. In other embodiments, the prostate tissue expresses AR and CK8 in the luminal layer. In further embodiments, the prostate tissue expresses Probasin or PSA. In one embodiment, the bladder tissue expresses CK8 in the luminal layer and uroplakins. In yet other embodiments, the bladder tissue stains positive for the presence of the sub-epithelial connective tissue layer (lamina propria) surrounding the urothelium with Gomori's trichrome. In some embodiments, the retrovirus is a lentivirus. In another embodiment, the lentivirus is doxycycline regulated.

An aspect of the invention is directed to an isolated population of induced epithelial cells obtained from the method described herein. In one embodiment, the cells express cytokeratin 5 (CK5), CK8, CK14, CK18, beta-catenin, E-cadherin, or a combination of the listed markers.

An aspect of the invention is directed to a method for transdifferentiation of embryonic fibroblast cells into an organ tissue, the method comprising: (a) isolating embryonic fibroblasts (EFs); (b) transducing EFs with a retrovirus comprising a reprogramming factor; (c) culturing the infected EFs in stem cell media for at least 24 hours at about 37° C. to generate induced pluripotent stem cells (iPSCs); (d) isolating iPSCs; (e) recombining the cells of (d) with mesenchymal cells; and (f) performing a graft of the recombined cells of (e) into an immunodeficient subject. In one embodiment, the stem cell media comprises LIF. In one embodiment, the graft is maintained in the subject for about 6 to 8 weeks. In one embodiment, the mesenchymal cells comprise urogenital mesenchyme. In one embodiment, the mesenchymal cells comprise bladder mesenchyme. In one embodiment, the graft is a renal graft. In one embodiment, the organ tissue is prostate epithelial tissue. In one embodiment, the organ tissue is bladder epithelial tissue. In one embodiment, the prostate tissue expresses p63, CK5, or a combination thereof, in the basal layer. In one embodiment, the bladder tissue expresses p63, CK5, or a combination thereof, in the basal layer. In one embodiment, the prostate tissue expresses AR, CK8, or a combination thereof, in the luminal layer. In one embodiment, the prostate tissue expresses Probasin, PSA, or a combination thereof. In one embodiment, the bladder tissue expresses CK8, uroplakins, or a combination thereof. In one embodiment, the bladder tissue stains positive for the presence of the sub-epithelial connective tissue layer (lamina propria) surrounding the urothelium with Gomori's trichrome. In one embodiment, the retrovirus is a lentivirus. In one embodiment, the lentivirus is doxycycline regulated.

An aspect of the invention is directed to a method for differentiation of induced pluripotent stem cells (iPSCs) into an organ tissue, the method comprising: (a) isolating iPSCs; (b) recombining the cells of (a) with mesenchymal cells; and (c) performing a graft of the recombined cells of (b) into an immunodeficient subject. In one embodiment, the graft is maintained in the subject for about 6 to 8 weeks. In one embodiment, the mesenchymal cells comprise urogenital mesenchyme. In one embodiment, the mesenchymal cells comprise bladder mesenchyme. In one embodiment, the graft is a renal graft. In one embodiment, the organ tissue is prostate epithelial tissue. In one embodiment, the organ tissue is bladder epithelial tissue. In one embodiment, the prostate tissue expresses p63, CK5, or a combination thereof, in the basal layer. In one embodiment, the bladder tissue expresses p63, CK5, or a combination thereof, in the basal layer. In one embodiment, the prostate tissue expresses AR, CK8, or a combination thereof, in the luminal layer. In one embodiment, the prostate tissue expresses Probasin, PSA, or a combination thereof. In one embodiment, the bladder tissue expresses CK8, uroplakins, or a combination thereof. In one embodiment, the bladder tissue stains positive for the presence of the sub-epithelial connective tissue layer (lamina propria) surrounding the urothelium with Gomori's trichrome.

An aspect of the invention is directed to a method for differentiation of induced pluripotent stem cells (iPSCs) into an organ tissue, the method comprising: (a) isolating iPSCs; (b) culturing iPSCs in endodermal differentiation media; (c) isolating iPSCs that express an endodermal marker; (d) recombining the cells of (c) with mesenchymal cells; and (e) performing a graft of the recombined cells of (d) into an immunodeficient subject. In one embodiment, the endodermal differentiation media contains Activin A, Noggin, and a GSK3β inhibitor. In another embodiment, the endodermal marker is GATA6. In one embodiment, the iPSCs are cultured in a three-dimensional culture. In one embodiment, the iPSCs are cultured in Matrigel. In another embodiment, the graft is maintained in the subject for about 6 to 8 weeks. In another embodiment, the mesenchymal cells comprise urogenital mesenchyme. In another embodiment, the mesenchymal cells comprise bladder mesenchyme. In another embodiment, the graft is a renal graft. In another embodiment, the organ tissue is prostate epithelial tissue. In another embodiment, the organ tissue is bladder epithelial tissue. In another embodiment, the prostate tissue expresses p63, CK5, or a combination thereof, in the basal layer. In another embodiment, the bladder tissue expresses p63, CK5, or a combination thereof, in the basal layer. In another embodiment, the prostate tissue expresses AR, CK8, or a combination thereof, in the luminal layer. In another embodiment, the prostate tissue expresses Probasin, PSA, or a combination thereof. In another embodiment, the bladder tissue expresses CK8, uroplakins, or a combination thereof. In another embodiment, the bladder tissue stains positive for the presence of the sub-epithelial connective tissue layer (lamina propria) surrounding the urothelium with Gomori's trichrome.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1 is a schematic showing master regulator analysis of cancer initiation using the human prostate cancer interactome. The MARINa algorithm was used to identify transcription factors that are putative master regulators of the transition from normal prostate epithelium to prostate cancer. The resulting transcription factors were further analyzed to identify synergistic pairs. 52 pairs were identified using a synergy threshold of 0.05 in comparison of Gleason grade 6 and 7 tumors with adjacent normal tissue. Blue indicates down-regulated pairs, while red indicates up-regulated pairs.

FIGS. 2A-B show graphs depicting reprogrammed MEFs express epithelial markers. FIG. 2A shows MEFs derived from Nkx3.1-lacZ knock-in mice were sorted for CD140a+/CD11b−/EpCAM− cells to be used for reprogramming experiments (red box). FIG. 2B (left) shows MEFs derived from Nkx3.1-lacZ knock-in mice were analyzed for EpCAM and CD24 expression before reprogramming FIG. 2B (right) shows that after infection of these MEFS with retroviruses expressing Oct4, Sox2, Klf4, and c-Myc, and culture for 14 days in prostate basal medium, 39% of the cells were EpCAM+CD24+ (blue box), and were used for tissue recombination experiments.

FIGS. 3A-C show fluorescent photomicrographs of immunostaining for epithelial marker expression. MEFs derived from Nkx3.1-lacZ knock-in mice were infected with retroviruses expressing Oct4, Sox2, Klf4, and c-Myc, followed by culture in prostate basal medium for 14 days and flow-sorting for EpCAM+CD24+ cells. Cells were then replated and immunostained for the indicated markers (FIGS. 3A-C). In FIG. 3A, most cells do not co-express the basal marker CK5 and the luminal marker CK18.

FIGS. 4A-H show photomicrographs of immunostaining for epithelial and prostate markers expression. FIGS. 4A-F show induced primitive epithelial cells were further transduced with Nkx3.1 and AR and used in tissue recombination assays. At 6 weeks, the renal grafts were harvested and analyzed for histology and immunostained with the indicated makers. FIG. 4G shows that in used positive controls, prostate epithelial cells from a 4-month old male mouse generated prostatic tissue in renal graft recombs. FIG. 4H shows induced primitive epithelial cells produced teratomas composed 90% from keratin.

FIG. 5 shows the strategy for production of prostate tissue by direct conversion/transdifferentiation of fibroblasts.

FIGS. 6A-H show the generation and analysis of induced epithelial (iEpt) cells. FIGS. 6A-B show that, after infection of MEFS with retroviruses expressing Oct4, Sox2, Klf4, and c-Myc, and culture for 14 days in prostate basal medium, 39% of the cells were EpCAM⁺CD24⁺, whereas 0.4% of control MEFs were EpCAM⁺CD24⁺. FIG. 6C shows the morphology of iEpt cells. FIGS. 6D-E show iEpt cells that were immunostained for basal (CK5) and luminal (CK8, CK18) markers. Note that iEpt cells represent a heterogeneous population, with many cells expressing basal markers (arrowhead in D) or luminal markers (arrow in E), and some cells co-expressing basal and luminal markers (arrow in D). FIGS. 6F-G show that the majority of iEpt cells display positive immunostaining for the epithelial markers E-cadherin and β-catenin. FIG. 6H shows Human BJ fibroblasts form iEpt cells after lentiviral infection with doxycycline-regulatable OSKM, and express both CK5 and CK8.

FIGS. 7A-P show the generation of reprogrammed mouse prostate tissue in renal grafts. FIGS. 7A,C,E,G,I,K,M show control tissue recombinants using wild-type mouse prostate analyzed by hematoxylin-eosin staining (H&E), or by immunostaining with the indicated markers. FIGS. 7B,D,F,H,J,L,N show reprogrammed prostate tissue derived from MEFs infected with REBNA viruses expressing OSKM, followed by retroviruses expressing AR and Nkx3.1. Arrowheads in F,H indicate basal cells. FIGS. 7O-P show reprogrammed prostate tissue derived from MDFs with transient expression of OSKM from a doxycycline-regulated transgene, followed by infection with retroviruses expressing AR and Nkx3.1.

FIGS. 8A-H show the production of reprogrammed human prostate tissue. FIGS. 8A,C,E,G show normal human prostate immunostained for the indicated markers. FIGS. 8B,D,F,H show reprogrammed prostate tissue from human fibroblasts infected with doxycycline-regulated OSKM lentiviruses, followed by retroviruses expressing AR and NKX3.1. Arrowheads in B,D indicate basal cells.

FIGS. 9A-B shows the identification of master regulators of normal prostate differentiation. FIG. 9A shows the projection of target genes inferred to be induced (red bars) and repressed (blue bars) by the indicated MRs on the genome-wide expression signature of prostate development between E16.5 and P90. Shown at the left is the p-value for the enrichment analysis of each MR target genes on the signature, and the inferred MR differential activity (DA) and differential expression (DE). FIG. 9B shows the synergistic regulation of inferred targets for NKX3.1 and FOXA1. The color of the nodes is proportional to their differential expression, showing down-regulated genes in blue and up-regulated genes in red.

FIGS. 10A-D show TALEN-mediated gene targeting in human prostate epithelial cells and fibroblasts. FIG. 10A shows the correct insertion and expression of GFP transgene in the AAVS1 locus in RWPE-1 cells. FIG. 10B shows the sequence of both AAVS1 alleles in a targeted clone. The allele at top (SEQ ID NOS 27 and 28, respectively, in order of appearance) has multiple insertions and rearrangements, while the allele at bottom (SEQ ID NOS 29 and 30, respectively, in order of appearance) has a large deletion. TALEN binding sites are shown in green and purple, insertions in red, deletions by dashes. FIGS. 10C-D show the targeting of TP53 in human BJ fibroblasts. At 4 days after targeting, cells were treated with 1 μM adriamycin for 6 hours, followed by immunostaining for p53.

FIGS. 11A-B show the generation of inducible Nanog-CreER^{T2} transgenic mice. FIG. 11A shows the BAC recombineering used to insert CreER^{T2} into the Nanog locus. FIG. 11B shows Tomato expression analyzed by direct visualization in Nanog-CreER^{T2}; R26R-Tomato/+ pre-implantation embryos dissected at 3.5 dpc and cultured overnight in the presence of 1 μm 4-OHT.

FIGS. 12A-F show the production of reprogrammed mouse prostate tissue with lentiviral vectors. (FIG. 12A-F) Reprogrammed prostate tissue derived from MEFs infected with Dox-inducible lentiviruses expressing OSKM, followed by lentiviruses expressing human AR, Nkx3.1 and Foxa1. (FIG. 12A) Gross anatomy of a tissue recombinant containing induced prostate tissue at 8 weeks post-grafting. (FIG. 12B-C) H&E histology of the same tissue recombinant. (FIG. 12D-F) Immunostaining with the indicated markers of serial sections to B&C.

FIGS. 13A-F show the production of reprogrammed mouse bladder tissue. (FIG. 13A,C,E) Control wild-type urinary bladder analyzed by H&E or by immunostaining with the indicated markers. (FIG. 13B,D,F) Reprogrammed bladder tissue derived from MEFs infected with Dox-inducible lentiviruses expressing OSKM, followed by lentiviruses expressing KLF5.

FIGS. 14A-K Production of reprogrammed mouse prostate tissue from CK18CreERT2; R26-Tomato iPS cells. (FIG. 14A-D) CK18CreERT2; R26-Tomato MEFs reprogram to iPS through a CK18+ state which is marked by Tomato recombination in the presence of 4-OHT, Dox and LIF. Imaging at 6 days (FIG. 14A,B) and 11 days of Dox induction (FIG. 14C,D). (FIG. 14E,F) Tissue recombinant of Tomato+ iPS colonies and UGM. (FIG. 14G,H) H&E histology of the same renal graft. (FIG. 14I-K) Immunostaining with the indicated markers of the same renal graft.

FIGS. 15A-F Generation of endodermal progenitors in 3D-culture from GATA6CreERT2;R26r-caggYFP iPS. (FIG. 15 A,B) GATA6CreERT2;R26r-caggYFP iPS passage 2 generated from the corresponding MEFs after expression of Dox-inducible OSKM for 11 days. (FIG. 15 C,D). Gata6/YFP+ colonies form in endodermal differentiation media from GATA6CreERT2;R26r-caggYFP iPS. (FIG. 15E,F) Gata6/YFP+ grow as spheres in 3D epithelial culture conditions in the presence of DHT.

### DETAILED DESCRIPTION OF THE INVENTION

Stem cell biologists have sought to generate desired cell types by activating lineage-specific differentiation pathways in the context of pluripotent embryonic stem cells (ESC) or induced pluripotent stem cells (iPSC). The directed differentiation of many epithelial cell types from ESC or iPSC can be challenging, perhaps since they typically reside in heterogeneous tissues containing multiple epithelial cell types within a stromal microenvironment. To overcome this challenge, the invention provides for the use of appropriate cell culture systems as well as tissue recombination methods in which mesenchymal cells are supplied to promote differentiation.

There has also been interest in transdifferentiation as another method for the generation of desired cell types [A1, A2], starting from the original demonstration that MyoD can be a master regulator that can reprogram fibroblasts into muscle cells [A3]. Furthermore, the generation of iPSC by Yamanaka and colleagues through ectopic expression of four “pluripotency factors” (OSKM: Oct4, Sox2, Klf4, c-Myc) [A4] has caused a resurgence of interest in molecular mechanisms of transdifferentiation. Several studies have now demonstrated that expression of lineage-specific master regulators can promote direct conversion or transdifferentiation from one mature differentiated cell type into a distinct differentiated cell type in the apparent absence of an intermediate pluripotent state. For example, fibroblasts can be directly converted to neurons or cardiomyocytes in culture by expression of lineage-specific MR genes [A5-A9], while induction of the pluripotency gene Oct4 combined with cytokine treatment can generate hematopoietic progenitors [A10].

An alternative approach for direct conversion, which has been termed “primed conversion” or “indirect lineage conversion” [A1, A2], has been to use transient expression of pluripotency factors to induce a plastic developmental state permissive for transdifferentiation into desired cell fates after exposure to appropriate external cues, such as specific cell culture conditions [A11, A12]. Neural progenitors generated by this methodology can be expanded in culture and generate different neuronal and glial types after multiple passages [A12, A13]. Thus, pluripotency factors can induce an epigenetically unstable state that is responsive to environmental signals and can be directed to lineage-specific progenitors and differentiated derivatives. The combination of this approach with the expression of lineage-specific master regulators can provide additional specificity or higher efficiency of direct conversion.

For direct conversion approaches, the generation of entire tissue, not just specific cell types, is desirable. This can be accomplished for epithelial tissues by combining epithelial progenitors generated by transdifferentiation with mesenchymal/stromal tissue that is specific for the tissue of interest, thereby recapitulating normal processes of organogenesis. In the case of the prostate, this approach can take advantage of a classic assay for prostate formation involving tissue recombination with rodent embryonic urogenital mesenchyme and renal grafting [A14, A15], which has been used for several studies of prostate differentiation and stem cell function [A16-A21]. This assay has been used for analyses of prostate stem/progenitor cells [A20-A23], and has also shown that human ESC can generate prostate epithelium in the context of teratomas following tissue recombination [A24]. Furthermore, embryonic urogenital mesenchyme is known to have potent reprogramming activity in tissue recombination assays, being capable of respecifying a range of epithelial cell types, such as bladder, vaginal, and mammary gland, to prostate epithelium [A15, A25-A27]. The contribution of organ-specific mesenchyme in enforcing correct lineage-specification and expansion of tissue progenitors has also been recognized for directed differentiation from pluripotent stem cells in culture [A28]. Direct conversion or differentiation to appropriate stem/progenitor cells, such as the prostate luminal stem cells that have previously been identified [A20], can enhance the production of desired cell types of interest.

Systems Analysis of Lineage Specific Master Regulators

The success and efficiency of direct conversion/transdifferentiation approaches depend upon the identification of suitable lineage-specific master regulator (MR) genes that can drive the direct conversion process. Candidate gene approaches to identify such MRs have been used, often by starting with a list of 10-20 transcription factors known to be important in the development and/or differentiation of the cell type of interest. This methodology relies upon the existence of a considerable body of literature on the cell type/tissue of interest, and is not feasible for cell types/tissues that are less well understood.

Candidate MRs for direct conversion can be systematically identified using a systems biology approach. Until recently, the molecular mechanisms underlying cell fate specification have been investigated without the benefit of comprehensive maps of the regulatory interactions that control lineage-specific differentiation. Recent work has led to the development of a large repertoire of computational methods for dissecting the molecular interactions that define the regulatory logic of cells and tissues. Methods for the dissection of cell type-specific regulatory networks and for identification of drivers of both physiological and pathological biological processes can be used. These include methods to infer transcriptional (ARACNe [A29, A30]) and post-translational (MINDy [A31]) interactions from large mRNA profile datasets. The resulting regulatory networks can then be interrogated to identify MR genes whose activity is both necessary and sufficient to implement a specific physiologic or pathologic cell state [A32, A33]. For example, this approach elucidated the synergistic role of the transcription factors C/EBPβ and Stat3 in reprogramming neural stem cells along a mesenchymal lineage [A32], and of the Huwe1-n-Myc-D113 cascade in brain morphogenesis in vivo [A34]. Without being bound by theory, the availability of an appropriate interactome and of signatures representing the gene expression differences of a progenitor state versus a fully differentiated tissue/cell type of interest can allow inference of MR genes governing transitions between these states that can be experimentally validated [A32, A33].

These computational/systems can be used for the identification of MRs of biological processes of interest. This methodology is unbiased, as it does not rely upon prior biological knowledge from functional studies using molecular genetic approaches. Many systems-based approaches have used expression profiling to identify differentially expressed genes, with the premise that highly differentially expressed genes can be enriched for master regulators. In contrast, the MARINa algorithm identifies candidate MRs on the basis of the differential expression of their inferred targets, and consequently can identify MRs that are not themselves differentially expressed, but display differential activity, for example, as a result of post-transcriptional regulation or post-translational modification such as phosphorylation.

Cancer Modeling by Gene Targeting and its Application to Human Prostate Cancer

Genetically-engineered mouse models of cancer have led to advances in understanding the biological and molecular mechanisms of cancer initiation and progression. Genetically-engineered mice can be intrinsically limited as models of human disease due to lack of conservation of tissue morphology, physiological states, and/or molecular pathways and regulatory genes. It is fundamentally important to generate appropriate human cancer models, but, the creation of precise genetically-engineered models can be hampered by technical difficulties with gene targeting in human cells.

Reagents, including zinc-finger nucleases and TALE nucleases (TALENs), can be used as gene targeting methods in experimental systems that have previously not been amenable to such approaches [A35]. TALENs correspond to fusions of sequence-specific TALE DNA-binding domains with the FokI restriction endonuclease [A36, A37], and can be engineered to bind and create a double-stranded break at a specific DNA sequence of interest in genomic DNA. TALENs have technical advantages since TALENs of any desired target specificity can be readily generated from standard starting reagents [A38]. Such TALENs can be used to mutate target genes by small insertions/deletions generated by TALEN-mediated double-strand DNA cleavage followed by non-homologous end-joining, or can be used as the basis for homologous recombination using an insertion vector as is the case for gene targeting in mouse ESCs. TALENs can be used for genetic engineering of human cells using approaches that have been well-developed over the past twenty years for manipulation of mouse ESC. The TALEN methodology is high-efficiency (often able to target both alleles in a single targeting experiment), non-cytotoxic, and has minimal off-target effects [A36, A37].

TALEN-mediated gene targeting can be utilized for the generation of genetically-engineered human models of cancer by mutation of tumor suppressor genes. In combination with direct conversion to generate tissues/cell types of interest, TALEN-mediated targeting can be used in fibroblasts or directly converted progeny cells to mutate target genes, followed by generation of human tissue that is cancer-prone or is undergoing cancer initiation. Since there are histological and physiological differences between the rodent and human prostate that limit the applicability of mouse models, these methods can be used for the generation of models of human prostate cancer. Genetically-engineered human models of prostate cancer based on gene targeting do not currently exist. An existing model that uses human prostate cells for oncogene overexpression in renal grafts [A39] uses primary normal prostate epithelial cells, which are difficult to obtain and cannot be propagated for use in gene targeting approaches.

The availability of genetically-engineered human models of prostate cancer can allow for the direct experimental analysis of prostate cancer initiation. The early events of human prostate cancer formation are poorly understood, due to the general lack of availability of human prostate tissue from men prior to clinical presentation of the disease [A40]. It is unclear when clinically-significant prostate cancer actually arises. Although prostate tissue from men in the twenties and thirties can contain localized areas of prostatic intraepithelial neoplasia (PIN) and latent adenocarcinoma, it is unknown whether this latent prostate cancer actually progresses to give rise to clinically aggressive disease in much older men (discussed in [A40]). Instead, this latent disease may be related to low-grade prostate cancer (histological Gleason grade 6 and 7 (3+4)) that is considered indolent and does not generally require treatment, whereas more aggressive prostate cancer (Gleason grade 7 (4+3) and above) can have an entirely different origin. There can be different origins of human prostate cancer that can be clinically distinct in terms of outcome, and it is unknown whether these differences are related to the mutational events that occur in prostate cancer initiation.

The invention provides for a direct conversion approach that can generate an entire tissue, not just a desired cell type of interest. In some embodiments, a computational systems biology approach can be used for the comprehensive identification of master regulator genes to optimize the direct conversion process. This approach can be combined with new gene targeting methods for the generation of novel genetically-engineered models of human cancer. Without being bound by theory, these approaches can be utilized for the analysis of human prostate cancer, but can also be used to model tumorigenesis in other tissues, as well as other diseases. For example, issues of primary clinical importance can be addressed, such as the molecular mechanisms that underlie the initiation and progression of human prostate cancer as the basis for aggressive versus indolent disease.

The invention is directed to methods for generating induced organ tissues. For example, the invention is directed to methods for the directed differentiation of mouse induced pluripotent stem cells (iPSC). The invention is also directed to transdifferentiation of mouse fibroblasts into prostate and urinary bladder epithelium, which have considerable clinical relevance for the patient-specific generation of normal and transformed prostate and bladder tissue. In one embodiment, the invention provides for methods of generating prostate tissue. In another embodiment, the invention provides for methods of generating bladder tissue. In some embodiments, the tissue is generated in vivo.

The invention encompasses methods for reprogramming fibroblast cells in culture, which are able to generate generic epithelial cells therefrom. These “primitive” epithelial cells can serve as the starting point for epithelial tissue formation in vivo upon transduction with specific tissue master regulatory genes together with grafting or co-culture of appropriate inductive mesenchyme or mesenchymal cells. Such tissues obtained by reprogramming include, but are not limited to prostate, urinary bladder, mammary gland, lung, as well as others.

Early stages of human prostate cancer are androgen-driven and thus respond to androgen-ablation therapy. However, in most cases a relapse occurs as a castration-resistant disease, which is progressive, metastatic and invariably lethal. These findings render mouse studies focused on generating new tissue engineering technologies to investigate the early events of prostate tumorigenesis highly relevant for human disease. Another leading cause of mortality in both men and women is urinary bladder cancer. In 90% of the cases, bladder cancer presents as urothelial cell carcinomas. In most cases, the treatment involves removal of the bladder wall followed by reconstructive surgeries, cystoplasty usually involving colon epithelium. These interventions leave the patient with highly debilitating long-term problems. Although a superior alternative, obtaining healthy functional autologous bladder urothelium has proved a challenging objective.

In one embodiment, the invention encompasses understanding the pathways involved in cellular identity and plasticity, as well as for developing patient-specific cell-based therapies for prostate and bladder disease. This approach can allow for the analysis of human prostate cancer initiation and early progression through the oncogenic transformation of prostate tissue generated by reprogramming. For example, such methods can allow for the analysis of the molecular basis for the differences between indolent and aggressive prostate cancer, which is likely to be established by early events in cancer initiation and progression [49]. This could lead to detection of new early prognostic biomarkers and would offer a new solution for drug screening. Generating bladder urothelium could have a more direct clinical applicability in regenerative medicine for patients with highly debilitating bladder exstrophy or cancer surgeries who need cystoplasty. More generally, the ability to generate patient-specific epithelial cell types from tissues that are otherwise difficult to access would represent a major advance in personalized and regenerative medicine.

Based on recent reprogramming studies [1, 2], the inherent plasticity of readily-accessible fibroblasts can be exploited to generate specific tissues (such as prostate and bladder epithelia) through a combination of reprogramming factors and tissue specific master regulator genes. As discussed in the Examples herein, mouse embryonic fibroblasts can be directly converted into epithelial cells in culture following expression of reprogramming factors, in the absence of an intermediate pluripotent stage. Moreover, these induced epithelial cells are amenable to further terminal differentiation into prostatic or bladder tissue in vivo in tissue recombination assays.

The invention encompasses methods directed to differentiation of mouse induced pluripotent stem cells (iPSC) into prostate and bladder epithelium by activation of master regulator genes of normal prostate and bladder epithelium, identified by bioinformatic analysis of regulatory genetic networks for mouse and human prostate or available from previous studies on urinary bladder development [3]. Expression of putative master regulator genes for prostate and bladder epithelium identified computationally or by a candidate gene approach can enhance prostate and bladder-specific differentiation of iPSC in tissue recombination experiments. In one embodiment, iPSC derived from various genetic backgrounds can be differentiated into mature epithelia through a temporal series of growth factors, genetic manipulations and in vivo recombination assays to mimic embryonic prostate and bladder development.

The invention further encompasses methods directed to conversion of mouse fibroblasts into prostate and bladder epithelium by transient expression of pluripotency factors (Oct4, Sox2, Klf4, c-Myc) to promote the directed transdifferentiation of mouse embryonic fibroblasts (MEFs) and human fibroblasts to “primitive” epithelial cells (iEpi) without undergoing an intermediate pluripotent state. Epithelial cells can be further directed toward prostate or bladder fate through expression of tissue specific master regulators and a pro-epithelial culture system. In one embodiment, MEFs derived from various genetic backgrounds and human fibroblasts can be briefly exposed to the pluripotency factors followed by transduction with prostate or bladder specific factors and cultured in epithelial conditions. In another embodiment, specific cell culture conditions (e.g., three-dimensional culture in Matrigel, co-culture with stromal cells) or tissue recombination assays can enhance the differentiation of desired epithelial cell.

The proposed studies aim at generating new ways to obtain complex tissues in vivo with a direct applicability in regenerative medicine. The resulting system would allow for functional studies to investigate the molecular nature of prostate tumorigenesis initiation in various oncogenic set-ups, and could lead to discovery of patient-specific early prognostic markers. Eventually, iPSC- and transdifferentiation-derived human bladder tissue could be considered for transplantation-based therapies in congenital defects (such as bladder exstrophy) or organ rehabilitation following cancer surgeries.

Direct Transdifferentiation in Regenerative Medicine and Disease Modeling

Stem cell biologists have sought to generate desired cell types by recapitulation of normal lineage-specific differentiation pathways from a pluripotent embryonic stem cell (ESC) or induced pluripotent stem cell (iPSC). To date, however, the directed differentiation of many epithelial cell types from ESC or iPSC has been relatively challenging, perhaps since they typically reside in a tissue containing multiple epithelial cell types within a stromal microenvironment. To overcome this challenge, the invention provides for the use of appropriate cell culture systems as well as tissue recombination methods in which mesenchymal cells are supplied to promote differentiation. Directed differentiation to appropriate adult stem/progenitor cells, such as the prostate luminal stem cells previously identified [4], can enhance the production of desired cell types of interest.

Previous studies have shown that human ESC can undergo complex differentiation along an endodermal lineage to generate prostate epithelium following recombination with rodent embryonic urogenital mesenchyme (UGM) and renal grafting [5, 6]. Similar to prostate, proper bladder development is dependent on proper stromal-epithelial crosstalk and paracrine signaling [7-10]. Tissue recombination techniques were employed to recapitulate bladder epithelium formation. Thus, embryonic bladder mesenchyme (EBLM) induces bladder morphogenesis when grafted together with mouse ESC [11] or bone marrow derived mesenchymal stem cells in tissue recombination models [12].

Prostate and bladder represent two functionally different types of epithelia. While prostate tissue is essentially a secretory glandular epthelium, the bladder is lined by urothelium, a permeability barrier epithelium, surrounded by lamina propria and a smooth muscle layer [13]. However, they appear similar from the point of view of tissue remodeling. Both prostate and urinary bladder are hindgut endodermal derivatives. The prostate develops from the pelvic (middle part) of the urogenital sinus (UGS), while urinary bladder forms from the cranial end of the UGS. Moreover, urogenital sinus mesenchyme (UGM) reprogrammed adult bladder epithelium to transdifferentiate into glandular epithelium in tissue recombination and renal grafting experiments [14]. Without being bound by theory, bladder and prostate can share a common stem cell/progenitor that is controlled by different inductive mesenchyme [11].

The efficiency of directed differentiation of pluripotent stem cells could be enhanced by the expression of lineage-specific master regulator genes that specify cell types of interest and can promote their differentiation. Without being bound by theory, such regulators can be determined by a candidate gene approach, or can be systematically identified using an unbiased reversed engineering approach. The candidate gene approach has been developed to generate and interrogate genome-wide regulatory networks, or interactomes, for cell types and tissues of interest [15-17]. The availability of such interactomes together with gene signatures of the tissue/cell types of interest allows the identification of master regulator genes that govern transitions to the differentiated cell type of interest [18, 19].

In one embodiment, lineage-specific master regulators can be used as an alternative approach to promote direct transdifferentiation from a distinct mature differentiated cell type in the absence of an intermediate pluripotent state. For instance, expression of four master regulator genes is sufficient to promote pancreatic beta-cell differentiation in vivo, albeit at low frequencies [20]; fibroblasts can be directly converted to neurons or cardiomyocytes in culture by expression of lineage-specific master regulator genes [21-23]; induction of the pluripotency gene Oct4 combined with cytokine treatment can generate hematopoietic progenitors [24]; and specific combinations of factors (Hnf4α, Foxa1, Foxa3, Gata4) can generate in vitro functional and proliferative hepatocyte-like cells from mouse fibroblasts [25, 26]. Moreover, the general reprogramming approach can be modified to serve as a platform for transdifferentiation [2]. Thus, transient expression of the four “pluripotency factors” (Oct4, Sox2, Klf4, c-Myc) in fibroblasts can lead to a plastic developmental state permissive for transdifferentiation into desired cell fates after exposure to appropriate external cues [27, 28]. Neural progenitors generated by this methodology can be expanded in culture and generate different neuronal and glial types after multiple passages [28]. Thus, pluripotency factors can induce an epigenetically unstable state that is responsive to environmental signals and can be directed to lineage-specific progenitors and differentiated derivatives. Directed transdifferentiation approaches can potentially overcome inherent limitations in the use of pluripotent cells for personalized treatments or regenerative medicine, such as low yields of differentiated cells, the need to generate patient-specific iPSC, or persistence of tumorigenic pluripotent cells.

Master Regulators of Direct Reprogramming to Prostate and Bladder Epithelium

As part of the candidate gene approach, an embodiment of the invention encompasses investigating whether genes with known biological function in regulating the developmental processes related to prostate and bladder are also appropriate master regulators of direct reprogramming.

The prostate is a secretory tissue of endodermal origin whose function is regulated by male sex hormones. Gene inactivation studies in the mouse, stem cell tracing mouse models combined with organ culture and tissue recombination assays, have highlighted the essential roles of androgenic signaling, epithelial-stromal interactions and specific stem cell populations in directing prostate development and regeneration[29]. The androgen receptor (AR) signaling axis plays a critical role in the development, function and homeostasis of the prostate[30, 31]. Mouse Nkx3.1 homeobox gene is the earliest known marker of prostate epithelium during embryogenesis and is subsequently expressed at all stages of prostate differentiation in vivo as well as in tissue recombinants. In the absence of Nkx3.1, the prostate ductal morphogenesis and secretory functions are disrupted [32]. Previous studies have placed the homeobox gene Nkx3.1, an important known regulator of prostate epithelial differentiation, at the center of prostate tissue homeostasis as a marker of a stem cell population active during prostate regeneration[29]. Based on genetic lineage-tracing analyses in mouse models, this work has shown that prostate stem cells reside among the Nkx3.1-positive luminal population, are castration resistant (Castration-resistant Nkx3.1-expressing cells, CARNs) and are able to regenerate prostatic glandular tissue after castration in an androgen-dependent manner [29]. Mouse Foxa1 expression marks the entire embryonic urogenital sinus epithelium (UGE), while Foxa2 is restricted to the basally located cells during prostate budding. Foxa1 plays a critical role in timing of prostate morphogenesis and cell differentiation. In Foxa1 deficient mice, the prostate has an abnormal ductal pattern composed of primitive epithelial cords surrounded by thick stromal layers [33]. Thus, the prostate epithelium development is blocked at a level similar to embryonic UGE and the primitive epithelial cells do not progress to differentiated and mature epithelial cells [33].

A recent study discussed the role for KLF5 in the formation and terminal differentiation of the urothelium [3]. When KLF5 is missing from the bladder epithelial cells, urothelial precursor cells remain in an undifferentiated state and the resulting urothelium fails to stratify and to express terminal differentiation markers (e.g. uroplakins). Moreover, the study uncovered and validated a plethora of transcriptional targets among the genes known to be coordinately expressed with KLF5 in the developing bladder: Pparγ, Grhl3, Ovol1, Foxa1, Elf3 and Ehf. Most importantly, Pparγ and Grhl3 participate in a KLF5-dependent gene network regulating maturation of the urothelium [3]. This study introduced order in the “black box” of the pathways involved in bladder development and opened the possibility that KLF5 could function as a master regulator of the reprogramming patterns in urothelium.

Without being bound by theory, focusing on a small number of core genes can significantly bias studies because other key players in determining epithelial tissue self-renewal and differentiation hierarchy would not be explored. An integrative systems biology approach can uncover whole gene pathways and networks, as well as new individual gene products which could be further validated experimentally. In one embodiment, the invention encompasses identifying and validating new master regulators (MRs) of epithelial reprogramming through unbiased genome-wide analysis of prostate and bladder urothelium.

Recent studies used powerful computational techniques of reverse-engineering designed to generate unbiased transcriptional and post-translational regulatory gene networks, or “interactomes” [17, 34]. These include an algorithm for the reconstruction of accurate cellular networks (ARACNe) [17], MARINa, for identification of most likely master regulators of specific expression signatures [18], MINDy, for the inference of post-transcriptional modulators of transcription factor activity [35], and master regulator analysis (MRA) [36]. These algorithms have accurately identified regulators of several human malignancies. Interrogation of a high-grade glioma interactome successfully identified two master regulator genes (C/EBPβ/δ and Stat3) that can reprogram neural stem cells along a mesenchymal lineage and that were validated both in vitro and in vivo [19]. In one embodiment, computational/systems biology approaches are used to construct genome-wide regulatory networks (interactomes) for mouse and human prostate tissue to allow identification of master regulator genes that govern prostate epithelial cell fates.

Methods for Isolating or Purifying Fibroblast Cells

The present invention provides methods for separating, enriching, isolating or purifying fibroblast cells from a tissue or mixed population of cells. The methods comprise obtaining a mixed population of cells, contacting the population of cells with an agent that binds to a mesenchymal marker, for example CD140a, and separating the subpopulation of cells that are bound by the agent from the subpopulation of cells that are not bound by the agent, wherein the subpopulation of cells that are bound by the agent is enriched for the mesenchymal marker (for example, CD140a-positive fibroblasts). The methods described herein may be performed using any mesenchymal marker known in the art, including, but not limited to N-cadherin (CD325), CD44, CD90, CD105, CD29, Sca-1, SSEA-4, vimentin, CD73, CD166, BMPR-1A, BMPR-1B, BMPR-II, CDCP1, fibronectin, CD49a, CD51, CD56, nestin, c-kit, STRO-1, and CD106.

The methods for separating, enriching, isolating or purifying fibroblast cells from a mixed population of cells according to the invention may be combined with other methods for separating, enriching, isolating or purifying fibroblast cells that are known in the art (for example, U.S. Pat. No. 4,777,145, U.S. Pat. No. 8,004,661, U.S. Pat. No. 5,367,474, U.S. Pat. No. 4,347,935) and are described in P. T. Sharpe, 1988, Laboratory Techniques in Biochemistry and Molecular Biology Volume 18: Methods of Cell Separation, Elsevier, Amsterdam; M. Zborowski and J. J. Chalmers, 2007, Laboratory Techniques in Biochemistry and Molecular Biology Volume 32: Magnetic Cell Separation, Elsevier, Amsterdam; and T. S. Hawley and R. G. Hawley, 2005, Methods in Molecular Biology Volume 263: Flow Cytometry Protocols, Humana Press Inc, Totowa, N.J. For example, the methods described herein may be performed in conjunction with techniques that use other markers. For example, additional selection steps maybe performed either before, after, or simultaneously with the mesenchymal marker selection step, in which a second agent, such as an antibody, that binds to a second marker is used, separating the subpopulation of cells that are bound by the agent from the subpopulation that are not bound by the agent, wherein the subpopulation of cells that are not bound by the agent is enriched. The second marker may be any marker known in the art that reduces the heterogeneity of the fibroblast population. For example, the second marker is the lineage surface antigens (Lin), Mac-1(CD11b), or epithelial cell adhesion molecule (EpCAM). In one embodiment, the second marker is a marker for blood cells (for example lineage surface antigens (Lin), Mac-1(CD11b), CD2, CD3, CD4, CD5, CD8, CD14, CD16, CD19, CD20, CD56, Ter119, B220, CD33, CD15, or CD45). In another embodiment, the second marker is a marker for endothelial cells (for example, CD34, CD146, CD202b, CD62e, CD54, VEGFR3, CD106, CD144, or CD309). In a further embodiment, the second marker is a marker for epithelial cells (for example, CD44R, CD66a, CD75, CD104, CD167, cytokeratin, EpCAM (CD326), CD138, or E-cadherin). In another embodiment, the second marker is a combination of any markers known in the art that reduce the heterogeneity of the fibroblast population (for example, Lin/Mac-1(CD11b)/EpCAM). The mixed population of cells can be any source of cells from which to obtain fibroblasts, including but not limited to an E13.5 mouse embryo, a P0 mouse, or a human foreskin. In one embodiment, mouse embryonic fibroblasts can be obtained from E13.5 mouse embryos. In another embodiment, mouse dermal fibroblasts can be obtained from P0 mice. In a further embodiment, BJ normal human foreskin fibroblasts can be obtained from human foreskins or from the American Type Culture Collection (for example cell line number CRL-2522).

The agent used can be any agent that binds to the mesenchymal marker (for example, CD140a), or the markers known in the art that reduce the heterogeneity of the fibroblast population (for example, Lin/Mac-1(CD11b)/EpCAM). The term “Agent” includes, but is not limited to small molecule drugs, peptides, proteins, peptidomimetic molecules, and antibodies. It also includes any molecule that binds to the mesenchymal marker, or to markers known in the art that reduce the heterogeneity of the fibroblast population, that is labeled with a detectable moiety, such as a histological stain, an enzyme substrate, a fluorescent moiety, a magnetic moiety or a radio-labeled moiety. Such “labeled” agents are particularly useful for embodiments involving isolation or purification of CD 140 positive cells, or detection of CD 140-positive cells, or isolation or purification of Lin/Mac-1(CD11b)/EpCAM negative cells. In some embodiments, the agent is an antibody that binds to CD140, Lin, Mac-1(CD11b), or EpCAM.

There are many cell separation techniques known in the art (U.S. Pat. No. 4,777,145, U.S. Pat. No. 8,004,661, U.S. Pat. No. 5,367,474, U.S. Pat. No. 4,347,935), and any such technique may be used. For example magnetic cell separation techniques can be used if the agent is labeled with an iron-containing moiety. Cells may also be passed over a solid support that has been conjugated to an agent that binds to a marker, such that the marker positive cells will be selectively retained on the solid support. Cells may also be separated by density gradient methods, particularly if the agent selected significantly increases the density of the marker positive cells to which it binds. For example, the agent can be a fluorescently labeled antibody against the marker, and the marker positive cells are separated from the other cells using fluorescence activated cell sorting (FACS).

DNA Manipulation for Reprogramming Factors and Master Regulatory Genes

One skilled in the art understands that polypeptides (for example Oct4, Sox2, Klf4, c-Myc, NKX3.1, Androgen receptor (AR), FOXA1, FOXA2, KLF5, Pparγ, Grhl3, Elf3, Ehf, and the like) can be obtained in several ways, which include but are not limited to, expressing a nucleotide sequence encoding the protein of interest by genetic engineering methods.

The invention provides for a nucleic acid encoding a reprogramming factor molecule, such as an Oct4 molecule, a Sox2 molecule, a Klf4 molecule, a c-Myc molecule, or a combination thereof. The invention further provides for a nucleic acid encoding a master regulatory molecule, such as a NKX3.1 molecule, an AR molecule, a FOXA1 molecule, a FOXA2 molecule, a KLF5 molecule, a Pparγ molecule, a Grhl3 molecule, a Elf3 molecule, a Ehf molecule, or a combination thereof. In one embodiment, the molecule (such as an Oct4 molecule, a Sox2 molecule, a Klf4 molecule, a c-Myc molecule, a NKX3.1 molecule, an AR molecule, a FOXA1 molecule, a FOXA2 molecule, a KLF5 molecule, a Pparγ molecule, a Grhl3 molecule, a Elf3 molecule, or a Ehf molecule) comprises an expression cassette, for example to achieve overexpression in a cell. The nucleic acids of the invention can be an RNA, cDNA, cDNA-like, or a DNA nucleic acid molecule of interest in an expressible format, such as an expression cassette, which can be expressed from the natural promoter or a derivative thereof or an entirely heterologous promoter. The nucleic acid of interest can encode a protein (for example, Oct4, Sox2, Klf4, c-Myc, NKX3.1, AR, FOXA1, FOXA2, KLF5, Pparγ, Grhl3, Elf3, or Ehf), and may or may not include introns. The nucleic acid of interest can encode only a single protein (for example, Oct4, Sox2, Klf4, c-Myc, NKX3.1, AR, FOXA1, FOXA2, KLF5, Pparγ, Grhl3, Elf3, or Ehf), or can encode for more than one protein of interest (for example, combinations of Oct4, Sox2, Klf4, c-Myc, NKX3.1, AR, FOXA1, FOXA2, KLF5, Pparγ, Grhl3, Elf3, or Ehf).

For example, the polypeptide sequence of human OCT4 (isoform 1) is depicted in SEQ ID NO: 1. OCT 4 is also known as POU5F1 (POU class 5 homeobox 1). The nucleotide sequence of human OCT4 (isoform 1) is shown in SEQ ID NO: 2. Sequence information related to OCT4 (isoform 1) is accessible in public databases by GenBank Accession numbers NP_{—}002692.2 (protein) and NM_{—}002701.4 (nucleic acid).

Sequence information related to OCT4 (isoform 2) is accessible in public databases by GenBank Accession numbers NP_{—}976034.4 (protein) and NM_{—}203289.4 (nucleic acid).

Sequence information related to OCT4 (transcript variant 3) is accessible in public databases by GenBank Accession numbers NP_{—}001167002.1 (protein) and NM_{—}001173531.1 (nucleic acid).

SEQ ID NO: 1 is the human wild type amino acid sequence corresponding to OCT4 isoform 1 (residues 1-360):

```
    1 MAGHLASDFA FSPPPGGGGD GPGGPEPGWV DPRTWLSFQG PPGGPGIGPG VGPGSEVWGI
  61 PPCPPPYEFC GGMAYCGPQV GVGLVPQGGL ETSQPEGEAG VGVESNSDGA SPEPCTVTPG
121 AVKLEKEKLE QNPEESQDIK ALQKELEQFA KLLKQKRITL GYTQADVGLT LGVLFGKVFS
181 QTTICRFEAL QLSFKNMCKL RPLLQKWVEE ADNNENLQEI CKAETLVQAR KRKRTSIENR
241 VRGNLENLFL QCPKPTLQQI SHIAQQLGLE KDVVRVWFCN RRQKGKRSSS DYAQREDFEA
301 AGSPFSGGPV SFPLAPGPHF GTPGYGSPHF TALYSSVPFP EGEAFPPVSV TTLGSPMHSN
```

SEQ ID NO: 2 is the human wild type nucleotide sequence corresponding to OCT4 (isoform 1) (nucleotides 1-1411), wherein the underscored bolded “ATG” denotes the beginning of the open reading frame:

```
      1 ccttcgcaag ccctcatttc accaggcccc cggcttgggg cgccttcctt ccccatggcg
    61 ggacacctgg cttcggattt cgccttctcg ccccctccag gtggtggagg tgatgggcca
  121 ggggggccgg agccgggctg ggttgatcct cggacctggc taagcttcca aggccctcct
  181 ggagggccag gaatcgggcc gggggttggg ccaggctctg aggtgtgggg gattccccca
  241 tgccccccgc cgtatgagtt ctgtgggggg atggcgtact gtgggcccca ggttggagtg
  301 gggctagtgc cccaaggcgg cttggagacc tctcagcctg agggcgaagc aggagtcggg
  361 gtggagagca actccgatgg ggcctccccg gagccctgca ccgtcacccc tggtgccgtg
  421 aagctggaga aggagaagct ggagcaaaac ccggaggagt cccaggacat caaagctctg
  481 cagaaagaac tcgagcaatt tgccaagctc ctgaagcaga agaggatcac cctgggatat
  541 acacaggccg atgtggggct caccctgggg gttctatttg ggaaggtatt cagccaaacg
  601 accatctgcc gctttgaggc tctgcagctt agcttcaaga acatgtgtaa gctgcggccc
  661 ttgctgcaga agtgggtgga ggaagctgac aacaatgaaa atcttcagga gatatgcaaa
  721 gcagaaaccc tcgtgcaggc ccgaaagaga aagcgaacca gtatcgagaa ccgagtgaga
  781 ggcaacctgg agaatttgtt cctgcagtgc ccgaaaccca cactgcagca gatcagccac
  841 atcgcccagc agcttgggct cgagaaggat gtggtccgag tgtggttctg taaccggcgc
  901 cagaagggca agcgatcaag cagcgactat gcacaacgag aggattttga ggctgctggg
  961 tctcctttct cagggggacc agtgtccttt cctctggccc cagggcccca ttttggtacc
1021 ccaggctatg ggagccctca cttcactgca ctgtactcct cggtcccttt ccctgagggg
1081 gaagcctttc cccctgtctc cgtcaccact ctgggctctc ccatgcattc aaactgaggt
1141 gcctgccctt ctaggaatgg gggacagggg gaggggagga gctagggaaa gaaaacctgg
1201 agtttgtgcc agggtttttg ggattaagtt cttcattcac taaggaagga attgggaaca
1261 caaagggtgg gggcagggga gtttggggca actggttgga gggaaggtga agttcaatga
1321 tgctcttgat tttaatccca catcatgtat cacttttttc ttaaataaag aagcctggga
1381 cacagtagat agacacactt aaaaaaaaaa a
```

For example, the polypeptide sequence of human SOX2 is depicted in SEQ ID NO: 3. The nucleotide sequence of human SOX2 is shown in SEQ ID NO: 4. Sequence information related to SOX2 is accessible in public databases by GenBank Accession numbers NP_{—}003097.1 (protein) and NM_{—}003106.3 (nucleic acid).

SEQ ID NO: 3 is the human wild type amino acid sequence corresponding to SOX2 (residues 1-317):

```
    1 MYNMMETELK PPGPQQTSGG GGGNSTAAAA GGNQKNSPDR VKRPMNAFMV WSRGQRRKMA
  61 QENPKMHNSE ISKRLGAEWK LLSETEKRPF IDEAKRLRAL HMKEHPDYKY RPRRKTKTLM
121 KKDKYTLPGG LLAPGGNSMA SGVGVGAGLG AGVNQRMDSY AHMNGWSNGS YSMMQDQLGY
181 PQHPGLNAHG AAQMQPMHRY DVSALQYNSM TSSQTYMNGS PTYSMSYSQQ GTPGMALGSM
241 GSVVKSEASS SPPVVTSSSH SRAPCQAGDL RDMISMYLPG AEVPEPAAPS RLHMSQHYQS
301 GPVPGTAING TLPLSHM
```

SEQ ID NO: 4 is the human wild type nucleotide sequence corresponding to SOX2 (nucleotides 1-2520), wherein the underscored bolded “ATG” denotes the beginning of the open reading frame:

```
      1 ggatggttgt ctattaactt gttcaaaaaa gtatcaggag ttgtcaaggc agagaagaga
    61 gtgtttgcaa aagggggaaa gtagtttgct gcctctttaa gactaggact gagagaaaga
  121 agaggagaga gaaagaaagg gagagaagtt tgagccccag gcttaagcct ttccaaaaaa
  181 taataataac aatcatcggc ggcggcagga tcggccagag gaggagggaa gcgctttttt
  241 tgatcctgat tccagtttgc ctctctcttt ttttccccca aattattctt cgcctgattt
  301 tcctcgcgga gccctgcgct cccgacaccc ccgcccgcct cccctcctcc tctccccccg
  361 cccgcgggcc ccccaaagtc ccggccgggc cgagggtcgg cggccgccgg cgggccgggc
  421 ccgcgcacag cgcccgcatg tacaacatga tggagacgga gctgaagccg ccgggcccgc
  481 agcaaacttc ggggggcggc ggcggcaact ccaccgcggc ggcggccggc ggcaaccaga
  541 aaaacagccc ggaccgcgtc aagcggccca tgaatgcctt catggtgtgg tcccgcgggc
  601 agcggcgcaa gatggcccag gagaacccca agatgcacaa ctcggagatc agcaagcgcc
  661 tgggcgccga gtggaaactt ttgtcggaga cggagaagcg gccgttcatc gacgaggcta
  721 agcggctgcg agcgctgcac atgaaggagc acccggatta taaataccgg ccccggcgga
  781 aaaccaagac gctcatgaag aaggataagt acacgctgcc cggcgggctg ctggcccccg
  841 gcggcaatag catggcgagc ggggtcgggg tgggcgccgg cctgggcgcg ggcgtgaacc
  901 agcgcatgga cagttacgcg cacatgaacg gctggagcaa cggcagctac agcatgatgc
  961 aggaccagct gggctacccg cagcacccgg gcctcaatgc gcacggcgca gcgcagatgc
1021 agcccatgca ccgctacgac gtgagcgccc tgcagtacaa ctccatgacc agctcgcaga
1081 cctacatgaa cggctcgccc acctacagca tgtcctactc gcagcagggc acccctggca
1141 tggctcttgg ctccatgggt tcggtggtca agtccgaggc cagctccagc ccccctgtgg
1201 ttacctcttc ctcccactcc agggcgccct gccaggccgg ggacctccgg gacatgatca
1261 gcatgtatct ccccggcgcc gaggtgccgg aacccgccgc ccccagcaga cttcacatgt
1321 cccagcacta ccagagcggc ccggtgcccg gcacggccat taacggcaca ctgcccctct
1381 cacacatgtg agggccggac agcgaactgg aggggggaga aattttcaaa gaaaaacgag
1441 ggaaatggga ggggtgcaaa agaggagagt aagaaacagc atggagaaaa cccggtacgc
1501 tcaaaaagaa aaaggaaaaa aaaaaatccc atcacccaca gcaaatgaca gctgcaaaag
1561 agaacaccaa tcccatccac actcacgcaa aaaccgcgat gccgacaaga aaacttttat
1621 gagagagatc ctggacttct ttttggggga ctatttttgt acagagaaaa cctggggagg
1681 gtggggaggg cgggggaatg gaccttgtat agatctggag gaaagaaagc tacgaaaaac
1741 tttttaaaag ttctagtggt acggtaggag ctttgcagga agtttgcaaa agtctttacc
1801 aataatattt agagctagtc tccaagcgac gaaaaaaatg ttttaatatt tgcaagcaac
1861 ttttgtacag tatttatcga gataaacatg gcaatcaaaa tgtccattgt ttataagctg
1921 agaatttgcc aatatttttc aaggagaggc ttcttgctga attttgattc tgcagctgaa
1981 atttaggaca gttgcaaacg tgaaaagaag aaaattattc aaatttggac attttaattg
2041 tttaaaaatt gtacaaaagg aaaaaattag aataagtact ggcgaaccat ctctgtggtc
2101 ttgtttaaaa agggcaaaag ttttagactg tactaaattt tataacttac tgttaaaagc
2161 aaaaatggcc atgcaggttg acaccgttgg taatttataa tagcttttgt tcgatcccaa
2221 ctttccattt tgttcagata aaaaaaacca tgaaattact gtgtttgaaa tattttctta
2281 tggtttgtaa tatttctgta aatttattgt gatattttaa ggttttcccc cctttatttt
2341 ccgtagttgt attttaaaag attcggctct gtattatttg aatcagtctg ccgagaatcc
2401 atgtatatat ttgaactaat atcatcctta taacaggtac attttcaact taagttttta
2461 ctccattatg cacagtttga gataaataaa tttttgaaat atggacactg aaaaaaaaaa
```

For example, the polypeptide sequence of human KLF4 is depicted in SEQ ID NO: 5. The nucleotide sequence of human KLF4 is shown in SEQ ID NO: 6. Sequence information related to KLF4 is accessible in public databases by GenBank Accession numbers NP_{—}004226.3 (protein) and NM_{—}004235.4 (nucleic acid).

SEQ ID NO: 5 is the human wild type amino acid sequence corresponding to KLF4 (residues 1-479):

```
    1 MRQPPGESDM AVSDALLPSF STFASGPAGR EKTLRQAGAP NNRWREELSH MKRLPPVLPG
  61 RPYDLAAATV ATDLESGGAG AACGGSNLAP LPRRETEEFN DLLDLDFILS NSLTHPPESV
121 AATVSSSASA SSSSSPSSSG PASAPSTCSF TYPIRAGNDP GVAPGGTGGG LLYGRESAPP
181 PTAPFNLADI NDVSPSGGFV AELLRPELDP VYIPPQQPQP PGGGLMGKFV LKASLSAPGS
241 EYGSPSVISV SKGSPDGSHP VVVAPYNGGP PRTCPKIKQE AVSSCTHLGA GPPLSNGHRP
301 AAHDFPLGRQ LPSRTTPTLG LEEVLSSRDC HPALPLPPGF HPHPGPNYPS FLPDQMQPQV
361 PPLHYQELMP PGSCMPEEPK PKRGRRSWPR KRTATHTCDY AGCGKTYTKS SHLKAHLRTH
421 TGEKPYHCDW DGCGWKFARS DELTRHYRKH TGHRPFQCQK CDRAFSRSDH LALHMKRHF
```

SEQ ID NO: 6 is the human wild type nucleotide sequence corresponding to KLF4 (nucleotides 1-2949), wherein the underscored bolded “ATG” denotes the beginning of the open reading frame:

```
      1 agtttcccga ccagagagaa cgaacgtgtc tgcgggcgcg cggggagcag aggcggtggc
    61 gggcggcggc ggcaccggga gccgccgagt gaccctcccc cgcccctctg gccccccacc
  121 ctcccacccg cccgtggccc gcgcccatgg ccgcgcgcgc tccacacaac tcaccggagt
  181 ccgcgccttg cgccgccgac cagttcgcag ctccgcgcca cggcagccag tctcacctgg
  241 cggcaccgcc cgcccaccgc cccggccaca gcccctgcgc ccacggcagc actcgaggcg
  301 accgcgacag tggtggggga cgctgctgag tggaagagag cgcagcccgg ccaccggacc
  361 tacttactcg ccttgctgat tgtctatttt tgcgtttaca acttttctaa gaacttttgt
  421 atacaaagga actttttaaa aaagacgctt ccaagttata tttaatccaa agaagaagga
  481 tctcggccaa tttggggttt tgggttttgg cttcgtttct tctcttcgtt gactttgggg
  541 ttcaggtgcc ccagctgctt cgggctgccg aggaccttct gggcccccac attaatgagg
  601 cagccacctg gcgagtctga catggctgtc agcgacgcgc tgctcccatc tttctccacg
  661 ttcgcgtctg gcccggcggg aagggagaag acactgcgtc aagcaggtgc cccgaataac
  721 cgctggcggg aggagctctc ccacatgaag cgacttcccc cagtgcttcc cggccgcccc
  781 tatgacctgg cggcggcgac cgtggccaca gacctggaga gcggcggagc cggtgcggct
  841 tgcggcggta gcaacctggc gcccctacct cggagagaga ccgaggagtt caacgatctc
  901 ctggacctgg actttattct ctccaattcg ctgacccatc ctccggagtc agtggccgcc
  961 accgtgtcct cgtcagcgtc agcctcctct tcgtcgtcgc cgtcgagcag cggccctgcc
1021 agcgcgccct ccacctgcag cttcacctat ccgatccggg ccgggaacga cccgggcgtg
1081 gcgccgggcg gcacgggcgg aggcctcctc tatggcaggg agtccgctcc ccctccgacg
1141 gctcccttca acctggcgga catcaacgac gtgagcccct cgggcggctt cgtggccgag
1201 ctcctgcggc cagaattgga cccggtgtac attccgccgc agcagccgca gccgccaggt
1261 ggcgggctga tgggcaagtt cgtgctgaag gcgtcgctga gcgcccctgg cagcgagtac
1321 ggcagcccgt cggtcatcag cgtcagcaaa ggcagccctg acggcagcca cccggtggtg
1381 gtggcgccct acaacggcgg gccgccgcgc acgtgcccca agatcaagca ggaggcggtc
1441 tcttcgtgca cccacttggg cgctggaccc cctctcagca atggccaccg gccggctgca
1501 cacgacttcc ccctggggcg gcagctcccc agcaggacta ccccgaccct gggtcttgag
1561 gaagtgctga gcagcaggga ctgtcaccct gccctgccgc ttcctcccgg cttccatccc
1621 cacccggggc ccaattaccc atccttcctg cccgatcaga tgcagccgca agtcccgccg
1681 ctccattacc aagagctcat gccacccggt tcctgcatgc cagaggagcc caagccaaag
1741 aggggaagac gatcgtggcc ccggaaaagg accgccaccc acacttgtga ttacgcgggc
1801 tgcggcaaaa cctacacaaa gagttcccat ctcaaggcac acctgcgaac ccacacaggt
1861 gagaaacctt accactgtga ctgggacggc tgtggatgga aattcgcccg ctcagatgaa
1921 ctgaccaggc actaccgtaa acacacgggg caccgcccgt tccagtgcca aaaatgcgac
1981 cgagcatttt ccaggtcgga ccacctcgcc ttacacatga agaggcattt ttaaatccca
2041 gacagtggat atgacccaca ctgccagaag agaattcagt attttttact tttcacactg
2101 tcttcccgat gagggaagga gcccagccag aaagcactac aatcatggtc aagttcccaa
2161 ctgagtcatc ttgtgagtgg ataatcagga aaaatgagga atccaaaaga caaaaatcaa
2221 agaacagatg gggtctgtga ctggatcttc tatcattcca attctaaatc cgacttgaat
2281 attcctggac ttacaaaatg ccaagggggt gactggaagt tgtggatatc agggtataaa
2341 ttatatccgt gagttggggg agggaagacc agaattccct tgaattgtgt attgatgcaa
2401 tataagcata aaagatcacc ttgtattctc tttaccttct aaaagccatt attatgatgt
2461 tagaagaaga ggaagaaatt caggtacaga aaacatgttt aaatagccta aatgatggtg
2521 cttggtgagt cttggttcta aaggtaccaa acaaggaagc caaagttttc aaactgctgc
2581 atactttgac aaggaaaatc tatatttgtc ttccgatcaa catttatgac ctaagtcagg
2641 taatatacct ggtttacttc tttagcattt ttatgcagac agtctgttat gcactgtggt
2701 ttcagatgtg caataatttg tacaatggtt tattcccaag tatgccttaa gcagaacaaa
2761 tgtgtttttc tatatagttc cttgccttaa taaatatgta atataaattt aagcaaacgt
2821 ctattttgta tatttgtaaa ctacaaagta aaatgaacat tttgtggagt ttgtattttg
2881 catactcaag gtgagaatta agttttaaat aaacctataa tattttatct gaaaaaaaaa
2941 aaaaaaaaa
```

For example, the polypeptide sequence of human c-MYC is depicted in SEQ ID NO: 7. c-MYC is also known as MYC. The nucleotide sequence of human c-MYC is shown in SEQ ID NO: 8. Sequence information related to c-MYC is accessible in public databases by GenBank Accession numbers NP_{—}002458.2 (protein) and NM_{—}002467.4 (nucleic acid).

SEQ ID NO: 7 is the human wild type amino acid sequence corresponding to c-MYC (residues 1-454):

```
    1 MDFFRVVENQ QPPATMPLNV SFTNRNYDLD YDSVQPYFYC DEEENFYQQQ QQSELQPPAP
  61 SEDIWKKFEL LPTPPLSPSR RSGLCSPSYV AVTPFSLRGD NDGGGGSFST ADQLEMVTEL
121 LGGDMVNQSF ICDPDDETFI KNIIIQDCMW SGFSAAAKLV SEKLASYQAA RKDSGSPNPA
181 RGHSVCSTSS LYLQDLSAAA SECIDPSVVF PYPLNDSSSP KSCASQDSSA FSPSSDSLLS
241 STESSPQGSP EPLVLHEETP PTTSSDSEEE QEDEEEIDVV SVEKRQAPGK RSESGSPSAG
301 GHSKPPHSPL VLKRCHVSTH QHNYAAPPST RKDYPAAKRV KLDSVRVLRQ ISNNRKCTSP
361 RSSDTEENVK RRTHNVLERQ RRNELKRSFF ALRDQIPELE NNEKAPKVVI LKKATAYILS
421 VQAEEQKLIS EEDLLRKRRE QLKHKLEQLR NSCA
```

SEQ ID NO: 8 is the human wild type nucleotide sequence corresponding to c-MYC (nucleotides 1-2379), wherein the underscored bolded “CTG” denotes the beginning of the open reading frame:

```
      1 gacccccgag ctgtgctgct cgcggccgcc accgccgggc cccggccgtc cctggctccc
    61 ctcctgcctc gagaagggca gggcttctca gaggcttggc gggaaaaaga acggagggag
  121 ggatcgcgct gagtataaaa gccggttttc ggggctttat ctaactcgct gtagtaattc
  181 cagcgagagg cagagggagc gagcgggcgg ccggctaggg tggaagagcc gggcgagcag
  241 agctgcgctg cgggcgtcct gggaagggag atccggagcg aatagggggc ttcgcctctg
  301 gcccagccct cccgctgatc ccccagccag cggtccgcaa cccttgccgc atccacgaaa
  361 ctttgcccat agcagcgggc gggcactttg cactggaact tacaacaccc gagcaaggac
  421 gcgactctcc cgacgcgggg aggctattct gcccatttgg ggacacttcc ccgccgctgc
  481 caggacccgc ttctctgaaa ggctctcctt gcagctgctt agacgctgga tttttttcgg
  541 gtagtggaaa accagcagcc tcccgcgacg atgcccctca acgttagctt caccaacagg
  601 aactatgacc tcgactacga ctcggtgcag ccgtatttct actgcgacga ggaggagaac
  661 ttctaccagc agcagcagca gagcgagctg cagcccccgg cgcccagcga ggatatctgg
  721 aagaaattcg agctgctgcc caccccgccc ctgtccccta gccgccgctc cgggctctgc
  781 tcgccctcct acgttgcggt cacacccttc tcccttcggg gagacaacga cggcggtggc
  841 gggagcttct ccacggccga ccagctggag atggtgaccg agctgctggg aggagacatg
  901 gtgaaccaga gtttcatctg cgacccggac gacgagacct tcatcaaaaa catcatcatc
  961 caggactgta tgtggagcgg cttctcggcc gccgccaagc tcgtctcaga gaagctggcc
1021 tcctaccagg ctgcgcgcaa agacagcggc agcccgaacc ccgcccgcgg ccacagcgtc
1081 tgctccacct ccagcttgta cctgcaggat ctgagcgccg ccgcctcaga gtgcatcgac
1141 ccctcggtgg tcttccccta ccctctcaac gacagcagct cgcccaagtc ctgcgcctcg
1201 caagactcca gcgccttctc tccgtcctcg gattctctgc tctcctcgac ggagtcctcc
1261 ccgcagggca gccccgagcc cctggtgctc catgaggaga caccgcccac caccagcagc
1321 gactctgagg aggaacaaga agatgaggaa gaaatcgatg ttgtttctgt ggaaaagagg
1381 caggctcctg gcaaaaggtc agagtctgga tcaccttctg ctggaggcca cagcaaacct
1441 cctcacagcc cactggtcct caagaggtgc cacgtctcca cacatcagca caactacgca
1501 gcgcctccct ccactcggaa ggactatcct gctgccaaga gggtcaagtt ggacagtgtc
1561 agagtcctga gacagatcag caacaaccga aaatgcacca gccccaggtc ctcggacacc
1621 gaggagaatg tcaagaggcg aacacacaac gtcttggagc gccagaggag gaacgagcta
1681 aaacggagct tttttgccct gcgtgaccag atcccggagt tggaaaacaa tgaaaaggcc
1741 cccaaggtag ttatccttaa aaaagccaca gcatacatcc tgtccgtcca agcagaggag
1801 caaaagctca tttctgaaga ggacttgttg cggaaacgac gagaacagtt gaaacacaaa
1861 cttgaacagc tacggaactc ttgtgcgtaa ggaaaagtaa ggaaaacgat tccttctaac
1921 agaaatgtcc tgagcaatca cctatgaact tgtttcaaat gcatgatcaa atgcaacctc
1981 acaaccttgg ctgagtcttg agactgaaag atttagccat aatgtaaact gcctcaaatt
2041 ggactttggg cataaaagaa cttttttatg cttaccatct tttttttttc tttaacagat
2101 ttgtatttaa gaattgtttt taaaaaattt taagatttac acaatgtttc tctgtaaata
2161 ttgccattaa atgtaaataa ctttaataaa acgtttatag cagttacaca gaatttcaat
2221 cctagtatat agtacctagt attataggta ctataaaccc taattttttt tatttaagta
2281 cattttgctt tttaaagttg atttttttct attgttttta gaaaaaataa aataactggc
2341 aaatatatca ttgagccaaa tcttaaaaaa aaaaaaaaa
```

For example, the polypeptide sequence of human NKX3.1 (isoform 1) is depicted in SEQ ID NO: 9. The nucleotide sequence of human NKX3.1 (isoform 1) is shown in SEQ ID NO: 10. Sequence information related to NKX3.1 (isoform 1) is accessible in public databases by GenBank Accession numbers NP_{—}006158.2 (protein) and NM_{—}006167.3 (nucleic acid).

Sequence information related to NKX3.1 (isoform 2) is accessible in public databases by GenBank Accession numbers NP_{—}1243268.1 (protein) and NM_{—}1256339.1 (nucleic acid).

SEQ ID NO: 9 is the human wild type amino acid sequence corresponding to NKX3.1 (isoform 1) (residues 1-234):

```
    1 MLRVPEPRPG EAKAEGAAPP TPSKPLTSFL IQDILRDGAQ RQGGRTSSQR QRDPEPEPEP
  61 EPEGGRSRAG AQNDQLSTGP RAAPEEAETL AETEPERHLG SYLLDSENTS GALPRLPQTP
121 KQPQKRSRAA FSHTQVIELE RKFSHQKYLS APERAHLAKN LKLTETQVKI WFQNRRYKTK
181 RKQLSSELGD LEKHSSLPAL KEEAFSRASL VSVYNSYPYY PYLYCVGSWS PAFW
```

SEQ ID NO: 10 is the human wild type nucleotide sequence corresponding to NKX3.1 (isoform 1) (nucleotides 1-3281), wherein the underscored bolded “ATG” denotes the beginning of the open reading frame:

```
      1 gcggtgcggg ccgggcgggt gcattcaggc caaggcgggg ccgccgggat gctcagggtt
    61 ccggagccgc ggcccgggga ggcgaaagcg gagggggccg cgccgccgac cccgtccaag
  121 ccgctcacgt ccttcctcat ccaggacatc ctgcgggacg gcgcgcagcg gcaaggcggc
  181 cgcacgagca gccagagaca gcgcgacccg gagccggagc cagagccaga gccagaggga
  241 ggacgcagcc gcgccggggc gcagaacgac cagctgagca ccgggccccg cgccgcgccg
  301 gaggaggccg agacgctggc agagaccgag ccagaaaggc acttggggtc ttatctgttg
  361 gactctgaaa acacttcagg cgcccttcca aggcttcccc aaacccctaa gcagccgcag
  421 aagcgctccc gagctgcctt ctcccacact caggtgatcg agttggagag gaagttcagc
  481 catcagaagt acctgtcggc ccctgaacgg gcccacctgg ccaagaacct caagctcacg
  541 gagacccaag tgaagatatg gttccagaac agacgctata agactaagcg aaagcagctc
  601 tcctcggagc tgggagactt ggagaagcac tcctctttgc cggccctgaa agaggaggcc
  661 ttctcccggg cctccctggt ctccgtgtat aacagctatc cttactaccc atacctgtac
  721 tgcgtgggca gctggagccc agctttttgg taatgccagc tcaggtgaca accattatga
  781 tcaaaaactg ccttccccag ggtgtctcta tgaaaagcac aaggggccaa ggtcagggag
  841 caagaggtgt gcacaccaaa gctattggag atttgcgtgg aaatctcaga ttcttcactg
  901 gtgagacaat gaaacaacag agacagtgaa agttttaata cctaagtcat tcctccagtg
  961 catactgtag gtcatttttt ttgcttctgg ctacctgttt gaaggggaga gagggaaaat
1021 caagtggtat tttccagcac tttgtatgat tttggatgag ttgtacaccc aaggattctg
1081 ttctgcaact ccatcctcct gtgtcactga atatcaactc tgaaagagca aacctaacag
1141 gagaaaggac aaccaggatg aggatgtcac caactgaatt aaacttaagt ccagaagcct
1201 cctgttggcc ttggaatatg gccaaggctc tctctgtccc tgtaaaagag aggggcaaat
1261 agagagtctc caagagaacg ccctcatgct cagcacatat ttgcatggga gggggagatg
1321 ggtgggagga gatgaaaata tcagcttttc ttattccttt ttattccttt taaaatggta
1381 tgccaactta agtatttaca gggtggccca aatagaacaa gatgcactcg ctgtgatttt
1441 aagacaagct gtataaacag aactccactg caagaggggg ggccgggcca ggagaatctc
1501 cgcttgtcca agacaggggc ctaaggaggg tctccacact gctgctaggg gctgttgcat
1561 ttttttatta gtagaaagtg gaaaggcctc ttctcaactt ttttcccttg ggctggagaa
1621 tttagaatca gaagtttcct ggagttttca ggctatcata tatactgtat cctgaaaggc
1681 aacataattc ttccttccct ccttttaaaa ttttgtgttc ctttttgcag caattactca
1741 ctaaagggct tcattttagt ccagattttt agtctggctg cacctaactt atgcctcgct
1801 tatttagccc gagatctggt cttttttttt tttttttttt ttttttttcc gtctccccaa
1861 agctttatct gtcttgactt tttaaaaaag tttgggggca gattctgaat tggctaaaag
1921 acatgcattt ttaaaactag caactcttat ttctttcctt taaaaataca tagcattaaa
1981 tcccaaatcc tatttaaaga cctgacagct tgagaaggtc actactgcat ttataggacc
2041 ttctggtggt tctgctgtta cgtttgaagt ctgacaatcc ttgagaatct ttgcatgcag
2101 aggaggtaag aggtattgga ttttcacaga ggaagaacac agcgcagaat gaagggccag
2161 gcttactgag ctgtccagtg gagggctcat gggtgggaca tggaaaagaa ggcagcctag
2221 gccctgggga gcccagtcca ctgagcaagc aagggactga gtgagccttt tgcaggaaaa
2281 ggctaagaaa aaggaaaacc attctaaaac acaacaagaa actgtccaaa tgctttggga
2341 actgtgttta ttgcctataa tgggtcccca aaatgggtaa cctagacttc agagagaatg
2401 agcagagagc aaaggagaaa tctggctgtc cttccatttt cattctgtta tctcaggtga
2461 gctggtagag gggagacatt agaaaaaaat gaaacaacaa aacaattact aatgaggtac
2521 gctgaggcct gggagtctct tgactccact acttaattcc gtttagtgag aaacctttca
2581 attttctttt attagaaggg ccagcttact gttggtggca aaattgccaa cataagttaa
2641 tagaaagttg gccaatttca ccccattttc tgtggtttgg gctccacatt gcaatgttca
2701 atgccacgtg ctgctgacac cgaccggagt actagccagc acaaaaggca gggtagcctg
2761 aattgctttc tgctctttac atttctttta aaataagcat ttagtgctca gtccctactg
2821 agtactcttt ctctcccctc ctctgaattt aattctttca acttgcaatt tgcaaggatt
2881 acacatttca ctgtgatgta tattgtgttg caaaaaaaaa aaaaaagtgt ctttgtttaa
2941 aattacttgg tttgtgaatc catcttgctt tttccccatt ggaactagtc attaacccat
3001 ctctgaactg gtagaaaaac atctgaagag ctagtctatc agcatctgac aggtgaattg
3061 gatggttctc agaaccattt cacccagaca gcctgtttct atcctgttta ataaattagt
3121 ttgggttctc tacatgcata acaaaccctg ctccaatctg tcacataaaa gtctgtgact
3181 tgaagtttag tcagcacccc caccaaactt tatttttcta tgtgtttttt gcaacatatg
3241 agtgttttga aaataaagta cccatgtctt tattagattt a
```

For example, the polypeptide sequence of human AR (Androgen Receptor) (isoform 1) is depicted in SEQ ID NO: 11. The nucleotide sequence of human AR (isoform 1) is shown in SEQ ID NO: 12. Sequence information related to AR (isoform 1) is accessible in public databases by GenBank Accession numbers NP_{—}000035.2 (protein) and NM_{—}000044.3 (nucleic acid).

Sequence information related to AR (isoform 2) is accessible in public databases by GenBank Accession numbers NP_{—}1011645.1 (protein) and NM_{—}10111645.2 (nucleic acid).

SEQ ID NO: 11 is the human wild type amino acid sequence corresponding to AR (isoform 1) (residues 1-920):

```
    1 MEVQLGLGRV YPRPPSKTYR GAFQNLFQSV REVIQNPGPR HPEAASAAPP GASLLLLQQQ
  61 QQQQQQQQQQ QQQQQQQQQQ ETSPRQQQQQ QGEDGSPQAH RRGPTGYLVL DEEQQPSQPQ
121 SALECHPERG CVPEPGAAVA ASKGLPQQLP APPDEDDSAA PSTLSLLGPT FPGLSSCSAD
181 LKDILSEAST MQLLQQQQQE AVSEGSSSGR AREASGAPTS SKDNYLGGTS TISDNAKELC
241 KAVSVSMGLG VEALEHLSPG EQLRGDCMYA PLLGVPPAVR PTPCAPLAEC KGSLLDDSAG
301 KSTEDTAEYS PFKGGYTKGL EGESLGCSGS AAAGSSGTLE LPSTLSLYKS GALDEAAAYQ
361 SRDYYNFPLA LAGPPPPPPP PHPHARIKLE NPLDYGSAWA AAAAQCRYGD LASLHGAGAA
421 GPGSGSPSAA ASSSWHTLFT AEEGQLYGPC GGGGGGGGGG GGGGGGGGGG GGGEAGAVAP
481 YGYTRPPQGL AGQESDFTAP DVWYPGGMVS RVPYPSPTCV KSEMGPWMDS YSGPYGDMRL
541 ETARDHVLPI DYYFPPQKTC LICGDEASGC HYGALTCGSC KVFFKRAAEG KQKYLCASRN
601 DCTIDKFRRK NCPSCRLRKC YEAGMTLGAR KLKKLGNLKL QEEGEASSTT SPTEETTQKL
661 TVSHIEGYEC QPIFLNVLEA IEPGVVCAGH DNNQPDSFAA LLSSLNELGE RQLVHVVKWA
721 KALPGFRNLH VDDQMAVIQY SWMGLMVFAM GWRSFTNVNS RMLYFAPDLV FNEYRMHKSR
781 MYSQCVRMRH LSQEFGWLQI TPQEFLCMKA LLLFSIIPVD GLKNQKFFDE LRMNYIKELD
841 RIIACKRKNP TSCSRRFYQL TKLLDSVQPI ARELHQFTFD LLIKSHMVSV DFPEMMAEII
901 SVQVPKILSG KVKPIYFHTQ
```

SEQ ID NO: 12 is the human wild type nucleotide sequence corresponding to AR (isoform 1) (nucleotides 1-10661), wherein the underscored bolded “ATG” denotes the beginning of the open reading frame:

```
        1 cgagatcccg gggagccagc ttgctgggag agcgggacgg tccggagcaa gcccagaggc
      61 agaggaggcg acagagggaa aaagggccga gctagccgct ccagtgctgt acaggagccg
    121 aagggacgca ccacgccagc cccagcccgg ctccagcgac agccaacgcc tcttgcagcg
    181 cggcggcttc gaagccgccg cccggagctg ccctttcctc ttcggtgaag tttttaaaag
    241 ctgctaaaga ctcggaggaa gcaaggaaag tgcctggtag gactgacggc tgcctttgtc
    301 ctcctcctct ccaccccgcc tccccccacc ctgccttccc cccctccccc gtcttctctc
    361 ccgcagctgc ctcagtcggc tactctcagc caacccccct caccaccctt ctccccaccc
    421 gcccccccgc ccccgtcggc ccagcgctgc cagcccgagt ttgcagagag gtaactccct
    481 ttggctgcga gcgggcgagc tagctgcaca ttgcaaagaa ggctcttagg agccaggcga
    541 ctggggagcg gcttcagcac tgcagccacg acccgcctgg ttaggctgca cgcggagaga
    601 accctctgtt ttcccccact ctctctccac ctcctcctgc cttccccacc ccgagtgcgg
    661 agccagagat caaaagatga aaaggcagtc aggtcttcag tagccaaaaa acaaaacaaa
    721 caaaaacaaa aaagccgaaa taaaagaaaa agataataac tcagttctta tttgcaccta
    781 cttcagtgga cactgaattt ggaaggtgga ggattttgtt tttttctttt aagatctggg
    841 catcttttga atctaccctt caagtattaa gagacagact gtgagcctag cagggcagat
    901 cttgtccacc gtgtgtcttc ttctgcacga gactttgagg ctgtcagagc gctttttgcg
    961 tggttgctcc cgcaagtttc cttctctgga gcttcccgca ggtgggcagc tagctgcagc
  1021 gactaccgca tcatcacagc ctgttgaact cttctgagca agagaagggg aggcggggta
  1081 agggaagtag gtggaagatt cagccaagct caaggatgga agtgcagtta gggctgggaa
  1141 gggtctaccc tcggccgccg tccaagacct accgaggagc tttccagaat ctgttccaga
  1201 gcgtgcgcga agtgatccag aacccgggcc ccaggcaccc agaggccgcg agcgcagcac
  1261 ctcccggcgc cagtttgctg ctgctgcagc agcagcagca gcagcagcag cagcagcagc
  1321 agcagcagca gcagcagcag cagcagcagc agcaagagac tagccccagg cagcagcagc
  1381 agcagcaggg tgaggatggt tctccccaag cccatcgtag aggccccaca ggctacctgg
  1441 tcctggatga ggaacagcaa ccttcacagc cgcagtcggc cctggagtgc caccccgaga
  1501 gaggttgcgt cccagagcct ggagccgccg tggccgccag caaggggctg ccgcagcagc
  1561 tgccagcacc tccggacgag gatgactcag ctgccccatc cacgttgtcc ctgctgggcc
  1621 ccactttccc cggcttaagc agctgctccg ctgaccttaa agacatcctg agcgaggcca
  1681 gcaccatgca actccttcag caacagcagc aggaagcagt atccgaaggc agcagcagcg
  1741 ggagagcgag ggaggcctcg ggggctccca cttcctccaa ggacaattac ttagggggca
  1801 cttcgaccat ttctgacaac gccaaggagt tgtgtaaggc agtgtcggtg tccatgggcc
  1861 tgggtgtgga ggcgttggag catctgagtc caggggaaca gcttcggggg gattgcatgt
  1921 acgccccact tttgggagtt ccacccgctg tgcgtcccac tccttgtgcc ccattggccg
  1981 aatgcaaagg ttctctgcta gacgacagcg caggcaagag cactgaagat actgctgagt
  2041 attccccttt caagggaggt tacaccaaag ggctagaagg cgagagccta ggctgctctg
  2101 gcagcgctgc agcagggagc tccgggacac ttgaactgcc gtctaccctg tctctctaca
  2161 agtccggagc actggacgag gcagctgcgt accagagtcg cgactactac aactttccac
  2221 tggctctggc cggaccgccg ccccctccgc cgcctcccca tccccacgct cgcatcaagc
  2281 tggagaaccc gctggactac ggcagcgcct gggcggctgc ggcggcgcag tgccgctatg
  2341 gggacctggc gagcctgcat ggcgcgggtg cagcgggacc cggttctggg tcaccctcag
  2401 ccgccgcttc ctcatcctgg cacactctct tcacagccga agaaggccag ttgtatggac
  2461 cgtgtggtgg tggtgggggt ggtggcggcg gcggcggcgg cggcggcggc ggcggcggcg
  2521 gcggcggcgg cggcgaggcg ggagctgtag ccccctacgg ctacactcgg ccccctcagg
  2581 ggctggcggg ccaggaaagc gacttcaccg cacctgatgt gtggtaccct ggcggcatgg
  2641 tgagcagagt gccctatccc agtcccactt gtgtcaaaag cgaaatgggc ccctggatgg
  2701 atagctactc cggaccttac ggggacatgc gtttggagac tgccagggac catgttttgc
  2761 ccattgacta ttactttcca ccccagaaga cctgcctgat ctgtggagat gaagcttctg
  2821 ggtgtcacta tggagctctc acatgtggaa gctgcaaggt cttcttcaaa agagccgctg
  2881 aagggaaaca gaagtacctg tgcgccagca gaaatgattg cactattgat aaattccgaa
  2941 ggaaaaattg tccatcttgt cgtcttcgga aatgttatga agcagggatg actctgggag
  3001 cccggaagct gaagaaactt ggtaatctga aactacagga ggaaggagag gcttccagca
  3061 ccaccagccc cactgaggag acaacccaga agctgacagt gtcacacatt gaaggctatg
  3121 aatgtcagcc catctttctg aatgtcctgg aagccattga gccaggtgta gtgtgtgctg
  3181 gacacgacaa caaccagccc gactcctttg cagccttgct ctctagcctc aatgaactgg
  3241 gagagagaca gcttgtacac gtggtcaagt gggccaaggc cttgcctggc ttccgcaact
  3301 tacacgtgga cgaccagatg gctgtcattc agtactcctg gatggggctc atggtgtttg
  3361 ccatgggctg gcgatccttc accaatgtca actccaggat gctctacttc gcccctgatc
  3421 tggttttcaa tgagtaccgc atgcacaagt cccggatgta cagccagtgt gtccgaatga
  3481 ggcacctctc tcaagagttt ggatggctcc aaatcacccc ccaggaattc ctgtgcatga
  3541 aagcactgct actcttcagc attattccag tggatgggct gaaaaatcaa aaattctttg
  3601 atgaacttcg aatgaactac atcaaggaac tcgatcgtat cattgcatgc aaaagaaaaa
  3661 atcccacatc ctgctcaaga cgcttctacc agctcaccaa gctcctggac tccgtgcagc
  3721 ctattgcgag agagctgcat cagttcactt ttgacctgct aatcaagtca cacatggtga
  3781 gcgtggactt tccggaaatg atggcagaga tcatctctgt gcaagtgccc aagatccttt
  3841 ctgggaaagt caagcccatc tatttccaca cccagtgaag cattggaaac cctatttccc
  3901 caccccagct catgccccct ttcagatgtc ttctgcctgt tataactctg cactactcct
  3961 ctgcagtgcc ttggggaatt tcctctattg atgtacagtc tgtcatgaac atgttcctga
  4021 attctatttg ctgggctttt tttttctctt tctctccttt ctttttcttc ttccctccct
  4081 atctaaccct cccatggcac cttcagactt tgcttcccat tgtggctcct atctgtgttt
  4141 tgaatggtgt tgtatgcctt taaatctgtg atgatcctca tatggcccag tgtcaagttg
  4201 tgcttgttta cagcactact ctgtgccagc cacacaaacg tttacttatc ttatgccacg
  4261 ggaagtttag agagctaaga ttatctgggg aaatcaaaac aaaaacaagc aaacaaaaaa
  4321 aaaaagcaaa aacaaaacaa aaaataagcc aaaaaacctt gctagtgttt tttcctcaaa
  4381 aataaataaa taaataaata aatacgtaca tacatacaca catacataca aacatataga
  4441 aatccccaaa gaggccaata gtgacgagaa ggtgaaaatt gcaggcccat ggggagttac
  4501 tgattttttc atctcctccc tccacgggag actttatttt ctgccaatgg ctattgccat
  4561 tagagggcag agtgacccca gagctgagtt gggcaggggg gtggacagag aggagaggac
  4621 aaggagggca atggagcatc agtacctgcc cacagccttg gtccctgggg gctagactgc
  4681 tcaactgtgg agcaattcat tatactgaaa atgtgcttgt tgttgaaaat ttgtctgcat
  4741 gttaatgcct cacccccaaa cccttttctc tctcactctc tgcctccaac ttcagattga
  4801 ctttcaatag tttttctaag acctttgaac tgaatgttct cttcagccaa aacttggcga
  4861 cttccacaga aaagtctgac cactgagaag aaggagagca gagatttaac cctttgtaag
  4921 gccccatttg gatccaggtc tgctttctca tgtgtgagtc agggaggagc tggagccaga
  4981 ggagaagaaa atgatagctt ggctgttctc ctgcttagga cactgactga atagttaaac
  5041 tctcactgcc actacctttt ccccaccttt aaaagacctg aatgaagttt tctgccaaac
  5101 tccgtgaagc cacaagcacc ttatgtcctc ccttcagtgt tttgtgggcc tgaatttcat
  5161 cacactgcat ttcagccatg gtcatcaagc ctgtttgctt cttttgggca tgttcacaga
  5221 ttctctgtta agagccccca ccaccaagaa ggttagcagg ccaacagctc tgacatctat
  5281 ctgtagatgc cagtagtcac aaagatttct taccaactct cagatcgctg gagcccttag
  5341 acaaactgga aagaaggcat caaagggatc aggcaagctg ggcgtcttgc ccttgtcccc
  5401 cagagatgat accctcccag caagtggaga agttctcact tccttcttta gagcagctaa
  5461 aggggctacc cagatcaggg ttgaagagaa aactcaatta ccagggtggg aagaatgaag
  5521 gcactagaac cagaaaccct gcaaatgctc ttcttgtcac ccagcatatc cacctgcaga
  5581 agtcatgaga agagagaagg aacaaagagg agactctgac tactgaatta aaatcttcag
  5641 cggcaaagcc taaagccaga tggacaccat ctggtgagtt tactcatcat cctcctctgc
  5701 tgctgattct gggctctgac attgcccata ctcactcaga ttccccacct ttgttgctgc
  5761 ctcttagtca gagggaggcc aaaccattga gactttctac agaaccatgg cttctttcgg
  5821 aaaggtctgg ttggtgtggc tccaatactt tgccacccat gaactcaggg tgtgccctgg
  5881 gacactggtt ttatatagtc ttttggcaca cctgtgttct gttgacttcg ttcttcaagc
  5941 ccaagtgcaa gggaaaatgt ccacctactt tctcatcttg gcctctgcct ccttacttag
  6001 ctcttaatct catctgttga actcaagaaa tcaagggcca gtcatcaagc tgcccatttt
  6061 aattgattca ctctgtttgt tgagaggata gtttctgagt gacatgatat gatccacaag
  6121 ggtttccttc cctgatttct gcattgatat taatagccaa acgaacttca aaacagcttt
  6181 aaataacaag ggagagggga acctaagatg agtaatatgc caatccaaga ctgctggaga
  6241 aaactaaagc tgacaggttc cctttttggg gtgggataga catgttctgg ttttctttat
  6301 tattacacaa tctggctcat gtacaggatc acttttagct gttttaaaca gaaaaaaata
  6361 tccaccactc ttttcagtta cactaggtta cattttaata ggtcctttac atctgttttg
  6421 gaatgatttt catcttttgt gatacacaga ttgaattata tcattttcat atctctcctt
  6481 gtaaatacta gaagctctcc tttacatttc tctatcaaat ttttcatctt tatgggtttc
  6541 ccaattgtga ctcttgtctt catgaatata tgtttttcat ttgcaaaagc caaaaatcag
  6601 tgaaacagca gtgtaattaa aagcaacaac tggattactc caaatttcca aatgacaaaa
  6661 ctagggaaaa atagcctaca caagccttta ggcctactct ttctgtgctt gggtttgagt
  6721 gaacaaagga gattttagct tggctctgtt ctcccatgga tgaaaggagg aggatttttt
  6781 ttttcttttg gccattgatg ttctagccaa tgtaattgac agaagtctca ttttgcatgc
  6841 gctctgctct acaaacagag ttggtatggt tggtatactg tactcacctg tgagggactg
  6901 gccactcaga cccacttagc tggtgagcta gaagatgagg atcactcact ggaaaagtca
  6961 caaggaccat ctccaaacaa gttggcagtg ctcgatgtgg acgaagagtg aggaagagaa
  7021 aaagaaggag caccagggag aaggctccgt ctgtgctggg cagcagacag ctgccaggat
  7081 cacgaactct gtagtcaaag aaaagagtcg tgtggcagtt tcagctctcg ttcattgggc
  7141 agctcgccta ggcccagcct ctgagctgac atgggagttg ttggattctt tgtttcatag
  7201 ctttttctat gccataggca atattgttgt tcttggaaag tttattattt ttttaactcc
  7261 cttactctga gaaagggata ttttgaagga ctgtcatata tctttgaaaa aagaaaatct
  7321 gtaatacata tatttttatg tatgttcact ggcactaaaa aatatagaga gcttcattct
  7381 gtcctttggg tagttgctga ggtaattgtc caggttgaaa aataatgtgc tgatgctaga
  7441 gtccctctct gtccatactc tacttctaaa tacatatagg catacatagc aagttttatt
  7501 tgacttgtac tttaagagaa aatatgtcca ccatccacat gatgcacaaa tgagctaaca
  7561 ttgagcttca agtagcttct aagtgtttgt ttcattaggc acagcacaga tgtggccttt
  7621 ccccccttct ctcccttgat atctggcagg gcataaaggc ccaggccact tcctctgccc
  7681 cttcccagcc ctgcaccaaa gctgcatttc aggagactct ctccagacag cccagtaact
  7741 acccgagcat ggcccctgca tagccctgga aaaataagag gctgactgtc tacgaattat
  7801 cttgtgccag ttgcccaggt gagagggcac tgggccaagg gagtggtttt catgtttgac
  7861 ccactacaag gggtcatggg aatcaggaat gccaaagcac cagatcaaat ccaaaactta
  7921 aagtcaaaat aagccattca gcatgttcag tttcttggaa aaggaagttt ctacccctga
  7981 tgcctttgta ggcagatctg ttctcaccat taatcttttt gaaaatcttt taaagcagtt
  8041 tttaaaaaga gagatgaaag catcacatta tataaccaaa gattacattg tacctgctaa
  8101 gataccaaaa ttcataaggg caggggggga gcaagcatta gtgcctcttt gataagctgt
  8161 ccaaagacag actaaaggac tctgctggtg actgacttat aagagctttg tgggtttttt
  8221 tttccctaat aatatacatg tttagaagaa ttgaaaataa tttcgggaaa atgggattat
  8281 gggtccttca ctaagtgatt ttataagcag aactggcttt ccttttctct agtagttgct
  8341 gagcaaattg ttgaagctcc atcattgcat ggttggaaat ggagctgttc ttagccactg
  8401 tgtttgctag tgcccatgtt agcttatctg aagatgtgaa acccttgctg ataagggagc
  8461 atttaaagta ctagattttg cactagaggg acagcaggca gaaatcctta tttctgccca
  8521 ctttggatgg cacaaaaagt tatctgcagt tgaaggcaga aagttgaaat acattgtaaa
  8581 tgaatatttg tatccatgtt tcaaaattga aatatatata tatatatata tatatatata
  8641 tatatatata tagtgtgtgt gtgtgttctg atagctttaa ctttctctgc atctttatat
  8701 ttggttccag atcacacctg atgccatgta cttgtgagag aggatgcagt tttgttttgg
  8761 aagctctctc agaacaaaca agacacctgg attgatcagt taactaaaag ttttctcccc
  8821 tattgggttt gacccacagg tcctgtgaag gagcagaggg ataaaaagag tagaggacat
  8881 gatacattgt actttactag ttcaagacag atgaatgtgg aaagcataaa aactcaatgg
  8941 aactgactga gatttaccac agggaaggcc caaacttggg gccaaaagcc tacccaagtg
  9001 attgaccagt ggccccctaa tgggacctga gctgttggaa gaagagaact gttccttggt
  9061 cttcaccatc cttgtgagag aagggcagtt tcctgcattg gaacctggag caagcgctct
  9121 atctttcaca caaattccct cacctgagat tgaggtgctc ttgttactgg gtgtctgtgt
  9181 gctgtaattc tggttttgga tatgttctgt aaagattttg acaaatgaaa atgtgttttt
  9241 ctctgttaaa acttgtcaga gtactagaag ttgtatctct gtaggtgcag gtccatttct
  9301 gcccacaggt agggtgtttt tctttgatta agagattgac acttctgttg cctaggacct
  9361 cccaactcaa ccatttctag gtgaaggcag aaaaatccac attagttact cctcttcaga
  9421 catttcagct gagataacaa atcttttgga attttttcac ccatagaaag agtggtagat
  9481 atttgaattt agcaggtgga gtttcatagt aaaaacagct tttgactcag ctttgattta
  9541 tcctcatttg atttggccag aaagtaggta atatgcattg attggcttct gattccaatt
  9601 cagtatagca aggtgctagg ttttttcctt tccccacctg tctcttagcc tggggaatta
  9661 aatgagaagc cttagaatgg gtggcccttg tgacctgaaa cacttcccac ataagctact
  9721 taacaagatt gtcatggagc tgcagattcc attgcccacc aaagactaga acacacacat
  9781 atccatacac caaaggaaag acaattctga aatgctgttt ctctggtggt tccctctctg
  9841 gctgctgcct cacagtatgg gaacctgtac tctgcagagg tgacaggcca gatttgcatt
  9901 atctcacaac cttagccctt ggtgctaact gtcctacagt gaagtgcctg gggggttgtc
  9961 ctatcccata agccacttgg atgctgacag cagccaccat cagaatgacc cacgcaaaaa
10021 aaagaaaaaa aaaattaaaa agtcccctca caacccagtg acacctttct gctttcctct
10081 agactggaac attgattagg gagtgcctca gacatgacat tcttgtgctg tccttggaat
10141 taatctggca gcaggaggga gcagactatg taaacagaga taaaaattaa ttttcaatat
10201 tgaaggaaaa aagaaataag aagagagaga gaaagaaagc atcacacaaa gattttctta
10261 aaagaaacaa ttttgcttga aatctcttta gatggggctc atttctcacg gtggcacttg
10321 gcctccactg ggcagcagga ccagctccaa gcgctagtgt tctgttctct ttttgtaatc
10381 ttggaatctt ttgttgctct aaatacaatt aaaaatggca gaaacttgtt tgttggacta
10441 catgtgtgac tttgggtctg tctctgcctc tgctttcaga aatgtcatcc attgtgtaaa
10501 atattggctt actggtctgc cagctaaaac ttggccacat cccctgttat ggctgcagga
10561 tcgagttatt gttaacaaag agacccaaga aaagctgcta atgtcctctt atcattgttg
10621 ttaatttgtt aaaacataaa gaaatctaaa atttcaaaaa a
```

For example, the polypeptide sequence of human FOXA1 is depicted in SEQ ID NO: 13. The nucleotide sequence of human FOXA1 is shown in SEQ ID NO: 14. Sequence information related to FOXA1 is accessible in public databases by GenBank Accession numbers NP_{—}004487.2 (protein) and NM_{—}004496.3 (nucleic acid).

SEQ ID NO: 13 is the human wild type amino acid sequence corresponding to FOXA1 (residues 1-472):

| | |
|---|---|
| 1 | MLGTVKMEGH ETSDWNSYYA DTQEAYSSVP VSNMNSGLGS |
| | MNSMNTYMTM NTMTTSGNMT |
| 61 | PASFNMSYAN PGLGAGLSPG AVAGMPGGSA GAMNSMTAAG |
| | VTAMGTALSP SGMGAMGAQQ |
| 121 | AASMNGLGPY AAAMNPCMSP MAYAPSNLGR SRAGGGGDAK |
| | TFKRSYPHAK PPYSYISLIT |
| 181 | MAIQQAPSKM LTLSEIYQWI MDLFPYYRQN QQRWQNSIRH |
| | SLSFNDCFVK VARSPDKPGK |
| 241 | GSYWTLHPDS GNMFENGCYL RRQKRFKCEK QPGAGGGGGS |
| | GSGGSGAKGG PESRKDPSGA |
| 301 | SNPSADSPLH RGVHGKTGQL EGAPAPGPAA SPQTLDHSGA |
| | TATGGASELK TPASSTAPPI |
| 361 | SSGPGALASV PASHPAHGLA PHESQLHLKG DPHYSFNHPF |
| | SINNLMSSSE QQHKLDFKAY |
| 421 | EQALQYSPYG STLPASLPLG SASVTTRSPI EPSALEPAYY |
| | QGVYSRPVLN TS |

SEQ ID NO: 14 is the human wild type nucleotide sequence corresponding to FOXA1 (nucleotides 1-3396), wherein the underscored bolded “ATG” denotes the beginning of the open reading frame:

| | |
|---|---|
| 1 | gggcttcctc ttcgcccggg tggcgttggg cccgcgcggg cgctcgggtg actgcagctg |
| 61 | ctcagctccc ctcccccgcc ccgcgccgcg cggccgcccg tcgcttcgca cagggctgga |
| 121 | tggttgtatt gggcagggtg gctccaggat gttaggaact gtgaagatgg aagggcatga |
| 181 | aaccagcgac tggaacagct actacgcaga cacgcaggag gcctactcct ccgtcccggt |
| 241 | cagcaacatg aactcaggcc tgggctccat gaactccatg aacacctaca tgaccatgaa |
| 301 | caccatgact acgagcggca acatgacccc ggcgtccttc aacatgtcct atgccaaccc |
| 361 | gggcctaggg gccggcctga gtcccggcgc agtagccggc atgccggggg gctcggcggg |
| 421 | cgccatgaac agcatgactg cggccggcgt gacggccatg ggtacggcgc tgagcccgag |
| 481 | cggcatgggc gccatgggtg cgcagcaggc ggcctccatg aatggcctgg gcccctacgc |
| 541 | ggccgccatg aacccgtgca tgagccccat ggcgtacgcg ccgtccaacc tgggccgcag |
| 601 | ccgcgcgggc ggcggcggcg acgccaagac gttcaagcgc agctacccgc acgccaagcc |
| 661 | gccctactcg tacatctcgc tcatcaccat ggccatccag caggcgccca gcaagatgct |
| 721 | cacgctgagc gagatctacc agtggatcat ggacctcttc ccctattacc ggcagaacca |
| 781 | gcagcgctgg cagaactcca tccgccactc gctgtccttc aatgactgct tcgtcaaggt |
| 841 | ggcacgctcc ccggacaagc cgggcaaggg ctcctactgg acgctgcacc cggactccgg |
| 901 | caacatgttc gagaacggct gctacttgcg ccgccagaag cgcttcaagt gcgagaagca |
| 961 | gccgggggcc ggcggcgggg gcgggagcgg aagcgggggc agcggcgcca agggcggccc |
| 1021 | tgagagccgc aaggacccct ctggcgcctc taaccccagc gccgactcgc ccctccatcg |
| 1081 | gggtgtgcac gggaagaccg gccagctaga gggcgcgccg gcccccgggc ccgccgccag |
| 1141 | cccccagact ctggaccaca gtggggcgac ggcgacaggg ggcgcctcgg agttgaagac |
| 1201 | tccagcctcc tcaactgcgc cccccataag ctccgggccc ggggcgctgg cctctgtgcc |
| 1261 | cgcctctcac ccggcacacg gcttggcacc ccacgagtcc cagctgcacc tgaaagggga |
| 1321 | cccccactac tccttcaacc acccgttctc catcaacaac ctcatgtcct cctcggagca |
| 1381 | gcagcataag ctggacttca aggcatacga acaggcactg caatactcgc cttacggctc |
| 1441 | tacgttgccc gccagcctgc ctctaggcag cgcctcggtg accaccagga gccccatcga |
| 1501 | gccctcagcc ctggagccgg cgtactacca aggtgtgtat tccagacccg tcctaaacac |
| 1561 | ttcctagctc ccgggactgg ggggtttgtc tggcatagcc atgctggtag caagagagaa |
| 1621 | aaaatcaaca gcaaacaaaa ccacacaaac caaaccgtca acagcataat aaaatcccaa |
| 1681 | caactatttt tatttcattt ttcatgcaca acctttcccc cagtgcaaaa gactgttact |
| 1741 | ttattattgt attcaaaatt cattgtgtat attactacaa agacaacccc aaaccaattt |
| 1801 | ttttcctgcg aagtttaatg atccacaagt gtatatatga aattctcctc cttccttgcc |
| 1861 | cccctctctt tcttccctct ttcccctcca gacattctag tttgtggagg gttatttaaa |
| 1921 | aaaacaaaaa aggaagatgg tcaagtttgt aaaatatttg tttgtgcttt ttccccctcc |
| 1981 | ttacctgacc ccctacgagt ttacaggtct gtggcaatac tcttaaccat aagaattgaa |
| 2041 | atggtgaaga aacaagtata cactagaggc tcttaaaagt attgaaagac aatactgctg |
| 2101 | ttatatagca agacataaac agattataaa catcagagcc atttgcttct cagtttacat |
| 2161 | ttctgataca tgcagatagc agatgtcttt aaatgaaata catgtatatt gtgtatggac |
| 2221 | ttaattatgc acatgctcag atgtgtagac atcctccgta tatttacata acatatagag |
| 2281 | gtaatagata ggtgatatac atgatacatt ctcaagagtt gcttgaccga aagttacaag |
| 2341 | gaccccaacc cctttgtcct ctctacccac agatggccct gggaatcaat tcctcaggaa |
| 2401 | ttgccctcaa gaactctgct tcttgctttg cagagtgcca tggtcatgtc attctgaggt |
| 2461 | cacataacac ataaaattag tttctatgag tgtataccat ttaaagaatt tttttttcag |
| 2521 | taaaagggaa tattacaatg ttggaggaga gataagttat agggagctgg atttcaaaac |
| 2581 | gtggtccaag attcaaaaat cctattgata gtggccattt taatcattgc catcgtgtgc |
| 2641 | ttgtttcatc cagtgttatg cactttccac agttggacat ggtgttagta tagccagacg |
| 2701 | ggtttcatta ttatttctct ttgctttctc aatgttaatt tattgcatgg tttattcttt |
| 2761 | ttctttacag ctgaaattgc tttaaatgat ggttaaaatt acaaattaaa ttgttaattt |
| 2821 | ttatcaatgt gattgtaatt aaaaatattt tgatttaaat aacaaaaata ataccagatt |
| 2881 | ttaagccgtg gaaaatgttc ttgatcattt gcagttaagg actttaaata aatcaaatgt |
| 2941 | taacaaaaga gcatttctgt tatttttttt cacttaacta aatccgaagt gaatatttct |
| 3001 | gaatacgata tttttcaaat tctagaactg aatataaatg acaaaaatga aaataaaatt |
| 3061 | gttttgtctg ttgttataat gaatgtgtag ctagtaaaaa ggagtgaaag aaattcaagt |
| 3121 | aaagtgtata agttgattta atattccaag agttgagatt tttaagattc tttattccca |
| 3181 | gtgatgttta cttcattttt tttttttttt ttgacaccgg cttaagcctt ctgtgtttcc |
| 3241 | tttgagcctt ttcactacaa aatcaaatat taatttaact acctttcctc cttccccaat |
| 3301 | gtatcacttt tctttatctg agaattcttc caatgaaaat aaaatatcag ctgtggctga |
| 3361 | tagaattaag ttgtgtccaa aaaaaaaaaa aaaaaa |

For example, the polypeptide sequence of human FOXA2 (isoform 1) is depicted in SEQ ID NO: 15. The nucleotide sequence of human FOXA2 (isoform 1) is shown in SEQ ID NO: 16. Sequence information related to FOXA2 (isoform 1) is accessible in public databases by GenBank Accession numbers NP_{—}068556.2 (protein) and NM_{—}021784.4 (nucleic acid).

Sequence information related to FOXA2 (isoform 2) is accessible in public databases by GenBank Accession numbers NP_{—}710141.1 (protein) and NM_{—}153675.2 (nucleic acid).

SEQ ID NO: 15 is the human wild type amino acid sequence corresponding to FOXA2 (isoform 1) (residues 1-463):

| | |
|---|---|
| 1 | MHSASSMLGA VKMEGHEPSD WSSYYAEPEG YSSVSNMNAG |
| | LGMNGMNTYM SMSAAAMGSG |
| 61 | SGNMSAGSMN MSSYVGAGMS PSLAGMSPGA GAMAGMGGSA |
| | GAAGVAGMGP HLSPSLSPLG |
| 121 | GQAAGAMGGL APYANMNSMS PMYGQAGLSR ARDPKTYRRS |
| | YTHAKPPYSY ISLITMAIQQ |
| 181 | SPNKMLTLSE IYQWIMDLFP FYRQNQQRWQ NSIRHSLSFN |
| | DCFLKVPRSP DKPGKGSFWT |
| 241 | LHPDSGNMFE NGCYLRRQKR FKCEKQLALK EAAGAAGSGK |
| | KAAAGAQASQ AQLGEAAGPA |
| 301 | SETPAGTESP HSSASPCQEH KRGGLGELKG TPAAALSPPE |
| | PAPSPGQQQQ AAAHLLGPPH |
| 361 | HPGLPPEAHL KPEHHYAFNH PFSINNLMSS EQQHHHSHHH |
| | HQPHKMDLKA YEQVMHYPGY |
| 421 | GSPMPGSLAM GPVTNKTGLD ASPLAADTSY YQGVYSRPIM |
| | NSS |

SEQ ID NO: 16 is the human wild type nucleotide sequence corresponding to FOXA2 (isoform 1) (nucleotides 1-2428), wherein the underscored bolded “ATG” denotes the beginning of the open reading frame:

| | |
|---|---|
| 1 | cccgcccact tccaactacc gcctccggcc tgcccaggga gagagaggga gtggagccca |
| 61 | gggagaggga gcgcgagaga gggagggagg aggggacggt gctttggctg actttttttt |
| 121 | aaaagagggt gggggtgggg ggtgattgct ggtcgtttgt tgtggctgtt aaattttaaa |
| 181 | ctgccatgca ctcggcttcc agtatgctgg gagcggtgaa gatggaaggg cacgagccgt |
| 241 | ccgactggag cagctactat gcagagcccg agggctactc ctccgtgagc aacatgaacg |
| 301 | ccggcctggg gatgaacggc atgaacacgt acatgagcat gtcggcggcc gccatgggca |
| 361 | gcggctcggg caacatgagc gcgggctcca tgaacatgtc gtcgtacgtg ggcgctggca |
| 421 | tgagcccgtc cctggcgggg atgtcccccg gcgcgggcgc catggcgggc atgggcggct |
| 481 | cggccggggc ggccggcgtg gcgggcatgg ggccgcactt gagtcccagc ctgagcccgc |
| 541 | tcggggggca ggcggccggg gccatgggcg gcctggcccc ctacgccaac atgaactcca |
| 601 | tgagccccat gtacgggcag gcgggcctga gccgcgcccg cgaccccaag acctacaggc |
| 661 | gcagctacac gcacgcaaag ccgccctact cgtacatctc gctcatcacc atggccatcc |
| 721 | agcagagccc caacaagatg ctgacgctga gcgagatcta ccagtggatc atggacctct |
| 781 | tccccttcta ccggcagaac cagcagcgct ggcagaactc catccgccac tcgctctcct |
| 841 | tcaacgactg tttcctgaag gtgccccgct cgcccgacaa gcccggcaag ggctccttct |
| 901 | ggaccctgca ccctgactcg ggcaacatgt tcgagaacgg ctgctacctg cgccgccaga |
| 961 | agcgcttcaa gtgcgagaag cagctggcgc tgaaggaggc cgcaggcgcc gccggcagcg |
| 1021 | gcaagaaggc ggccgccgga gcccaggcct cacaggctca actcggggag gccgccgggc |
| 1081 | cggcctccga gactccggcg ggcaccgagt cgcctcactc gagcgcctcc ccgtgccagg |
| 1141 | agcacaagcg agggggcctg ggagagctga aggggacgcc ggctgcggcg ctgagccccc |
| 1201 | cagagccggc gccctctccc gggcagcagc agcaggccgc ggcccacctg ctgggcccgc |
| 1261 | cccaccaccc gggcctgccg cctgaggccc acctgaagcc ggaacaccac tacgccttca |
| 1321 | accacccgtt ctccatcaac aacctcatgt cctcggagca gcagcaccac cacagccacc |
| 1381 | accaccacca accccacaaa atggacctca aggcctacga acaggtgatg cactaccccg |
| 1441 | gctacggttc ccccatgcct ggcagcttgg ccatgggccc ggtcacgaac aaaacgggcc |
| 1501 | tggacgcctc gcccctggcc gcagatacct cctactacca gggggtgtac tcccggccca |
| 1561 | ttatgaactc ctcttaagaa gacgacggct tcaggcccgg ctaactctgg caccccggat |
| 1621 | cgaggacaag tgagagagca agtgggggtc gagactttgg ggagacggtg ttgcagagac |
| 1681 | gcaagggaga agaaatccat aacaccccca ccccaacacc cccaagacag cagtcttctt |
| 1741 | cacccgctgc agccgttccg tcccaaacag agggccacac agatacccca cgttctatat |
| 1801 | aaggaggaaa acgggaaaga atataaagtt aaaaaaaagc ctccggtttc cactactgtg |
| 1861 | tagactcctg cttcttcaag cacctgcaga ttctgatttt tttgttgttg ttgttctcct |
| 1921 | ccattgctgt tgttgcaggg aagtcttact taaaaaaaaa aaaaaatttt gtgagtgact |
| 1981 | cggtgtaaaa ccatgtagtt ttaacagaac cagagggttg tactattgtt taaaaacagg |
| 2041 | aaaaaaaata atgtaagggt ctgttgtaaa tgaccaagaa aaagaaaaaa aaagcattcc |
| 2101 | caatcttgac acggtgaaat ccaggtctcg ggtccgatta atttatggtt tctgcgtgct |
| 2161 | ttatttatgg cttataaatg tgtattctgg ctgcaagggc cagagttcca caaatctata |
| 2221 | ttaaagtgtt atacccggtt ttatcccttg aatcttttct tccagatttt tcttttcttt |
| 2281 | acttggctta caaaatatac aggcttggaa attatttcaa gaaggaggga gggataccct |
| 2341 | gtctggttgc aggttgtatt ttattttggc ccagggagtg ttgctgtttt cccaacattt |
| 2401 | tattaataaa attttcagac ataaaaaa |

For example, the polypeptide sequence of human KLF5 is depicted in SEQ ID NO: 17. The nucleotide sequence of human KLF5 is shown in SEQ ID NO: 18. Sequence information related to KLF5 is accessible in public databases by GenBank Accession numbers NP_{—}001721.2 (protein) and NM_{—}001730.3 (nucleic acid).

SEQ ID NO: 17 is the human wild type amino acid sequence corresponding to KLF5 (residues 1-457):

| | |
|---|---|
| 1 | MATRVLSMSA RLGPVPQPPA PQDEPVFAQL KPVLGAANPA |
| | RDAALFPGEE LKHAHHRPQA |
| 61 | QPAPAQAPQP AQPPATGPRL PPEDLVQTRC EMEKYLTPQL |
| | PPVPIIPEHK KYRRDSASVV |
| 121 | DQFFTDTEGL PYSINMNVFL PDITHLRTGL YKSQRPCVTH |
| | IKTEPVAIFS HQSETTAPPP |
| 181 | APTQALPEFT SIFSSHQTAA PEVNNIFIKQ ELPTPDLHLS |
| | VPTQQGHLYQ LLNTPDLDMP |
| 241 | SSTNQTAAMD TLNVSMSAAM AGLNTHTSAV PQTAVKQFQG |
| | MPPCTYTMPS QFLPQQATYF |
| 301 | PPSPPSSEPG SPDRQAEMLQ NLTPPPSYAA TIASKLAIHN |
| | PNLPTTLPVN SQNIQPVRYN |
| 361 | RRSNPDLEKR RIHYCDYPGC TKVYTKSSHL KAHLRTHTGE |
| | KPYKCTWEGC DWRFARSDEL |
| 421 | TRHYRKHTGA KPFQCGVCNR SFSRSDHLAL HMKRHQN |

SEQ ID NO: 18 is the human wild type nucleotide sequence corresponding to KLF5 (nucleotides 1-3350), wherein the underscored bolded “ATG” denotes the beginning of the open reading frame:

| | |
|---|---|
| 1 | tagtcgcggg gcaggtacgt gcgctcgcgg ttctctcgcg gaggtcggcg gtggcgggag |
| 61 | cgggctccgg agagcctgag agcacggtgg ggcggggcgg gagaaagtgg ccgcccggag |
| 121 | gacgttggcg tttacgtgtg gaagagcgga agagttttgc ttttcgtgcg cgccttcgaa |
| 181 | aactgcctgc cgctgtctga ggagtccacc cgaaacctcc cctcctccgc cggcagcccc |
| 241 | gcgctgagct cgccgaccca agccagcgtg ggcgaggtgg gaagtgcgcc cgacccgcgc |
| 301 | ctggagctgc gcccccgagt gcccatggct acaagggtgc tgagcatgag cgcccgcctg |
| 361 | ggacccgtgc cccagccgcc ggcgccgcag gacgagccgg tgttcgcgca gctcaagccg |
| 421 | gtgctgggcg ccgcgaatcc ggcccgcgac gcggcgctct tccccggcga ggagctgaag |
| 481 | cacgcgcacc accgcccgca ggcgcagccc gcgcccgcgc aggccccgca gccggcccag |
| 541 | ccgcccgcca ccggcccgcg gctgcctcca gaggacctgg tccagacaag atgtgaaatg |
| 601 | gagaagtatc tgacacctca gcttcctcca gttcctataa ttccagagca taaaaagtat |
| 661 | agacgagaca gtgcctcagt cgtagaccag ttcttcactg acactgaagg gttaccttac |
| 721 | agtatcaaca tgaacgtctt cctccctgac atcactcacc tgagaactgg cctctacaaa |
| 781 | tcccagagac cgtgcgtaac acacatcaag acagaacctg ttgccatttt cagccaccag |
| 841 | agtgaaacga ctgcccctcc tccggccccg acccaggccc tccctgagtt caccagtata |
| 901 | ttcagctcac accagaccgc agctccagag gtgaacaata ttttcatcaa acaagaactt |
| 961 | cctacaccag atcttcatct ttctgtccct acccagcagg gccacctgta ccagctactg |
| 1021 | aatacaccgg atctagatat gcccagttct acaaatcaga cagcagcaat ggacactctt |
| 1081 | aatgtttcta tgtcagctgc catggcaggc cttaacacac acacctctgc tgttccgcag |
| 1141 | actgcagtga aacaattcca gggcatgccc ccttgcacat acacaatgcc aagtcagttt |
| 1201 | cttccacaac aggccactta ctttcccccg tcaccaccaa gctcagagcc tggaagtcca |
| 1261 | gatagacaag cagagatgct ccagaattta accccacctc catcctatgc tgctacaatt |
| 1321 | gcttctaaac tggcaattca caatccaaat ttacccacca ccctgccagt taactcacaa |
| 1381 | aacatccaac ctgtcagata caatagaagg agtaaccccg atttggagaa acgacgcatc |
| 1441 | cactactgcg attaccctgg ttgcacaaaa gtttatacca agtcttctca tttaaaagct |
| 1501 | cacctgagga ctcacactgg tgaaaagcca tacaagtgta cctgggaagg ctgcgactgg |
| 1561 | aggttcgcgc gatcggatga gctgacccgc cactaccgga agcacacagg cgccaagccc |
| 1621 | ttccagtgcg gggtgtgcaa ccgcagcttc tcgcgctctg accacctggc cctgcatatg |
| 1681 | aagaggcacc agaactgagc actgcccgtg tgacccgttc caggtcccct gggctccctc |
| 1741 | aaatgacaga cctaactatt cctgtgtaaa aacaacaaaa acaaacaaaa gcaagaaaac |
| 1801 | cacaactaaa actggaaatg tatattttgt atatttgaga aaacagggaa tacattgtat |
| 1861 | taataccaaa gtgtttggtc attttaagaa tctggaatgc ttgctgtaat gtatatggct |
| 1921 | ttactcaagc agatctcatc tcatgacagg cagccacgtc tcaacatggg taaggggtgg |
| 1981 | gggtggaggg gagtgtgtgc agcgttttta cctaggcacc atcatttaat gtgacagtgt |
| 2041 | tcagtaaaca aatcagttgg caggcaccag aagaagaatg gattgtatgt caagatttta |
| 2101 | cttggcattg agtagttttt ttcaatagta ggtaattcct tagagataca gtatacctgg |
| 2161 | caattcacaa atagccattg aacaaatgtg tgggttttta aaaattatat acatatatga |
| 2221 | gttgcctata tttgctattc aaaattttgt aaatatgcaa atcagcttta taggtttatt |
| 2281 | acaagttttt taggattctt ttggggaaga gtcataattc ttttgaaaat aaccatgaat |
| 2341 | acacttacag ttaggatttg tggtaaggta cctctcaaca ttaccaaaat catttcttta |
| 2401 | gagggaagga ataatcattc aaatgaactt taaaaaagca aatttcatgc actgattaaa |
| 2461 | ataggattat tttaaataca aaaggcattt tatatgaatt ataaactgaa gagcttaaag |
| 2521 | atagttacaa aatacaaaag ttcaacctct tacaataagc taaacgcaat gtcattttta |
| 2581 | aaaagaagga cttagggtgt cgttttcaca tatgacaatg ttgcatttat gatgcagttt |
| 2641 | caagtaccaa aacgttgaat tgatgatgca gttttcatat atcgagatgt tcgctcgtgc |
| 2701 | agtactgttg gttaaatgac aatttatgtg gattttgcat gtaatacaca gtgagacaca |
| 2761 | gtaattttat ctaaattaca gtgcagttta gttaatctat taatactgac tcagtgtctg |
| 2821 | cctttaaata taaatgatat gttgaaaact taaggaagca aatgctacat atatgcaata |
| 2881 | taaaatagta atgtgatgct gatgctgtta accaaagggc agaataaata agcaaaatgc |
| 2941 | caaaaggggt cttaattgaa atgaaaattt aattttgttt ttaaaatatt gtttatcttt |
| 3001 | atttattttg tggtaatata gtaagttttt ttagaagaca attttcataa cttgataaat |
| 3061 | tatagttttg tttgttagaa aagttgctct taaaagatgt aaatagatga caaacgatgt |
| 3121 | aaataatttt gtaagaggct tcaaaatgtt tatacgtgga aacacaccta catgaaaagc |
| 3181 | agaaatcggt tgctgttttg cttctttttc cctcttattt ttgtattgtg gtcatttcct |
| 3241 | atgcaaataa tggagcaaac agctgtatag ttgtagaatt ttttgagaga atgagatgtt |
| 3301 | tatatattaa cgacaatttt ttttttggaa aataaaaagt gcctaaaaga |

For example, the polypeptide sequence of human PPARγ (isoform 1, variant 1) is depicted in SEQ ID NO: 19. PPARγ is also known as PPARG. The nucleotide sequence of human PPARγ (isoform 1, variant 1) is shown in SEQ ID NO: 20. Sequence information related to PPARγ (isoform 1, variant 1) is accessible in public databases by GenBank Accession numbers NP_{—}619726.2 (protein) and NM_{—}138712.3 (nucleic acid).

Sequence information related to PPARγ (isoform 1, variant 3) is accessible in public databases by GenBank Accession numbers NP_{—}619725.2 (protein) and NM_{—}138711.3 (nucleic acid).

Sequence information related to PPARγ (isoform 1, variant 4) is accessible in public databases by GenBank Accession numbers NP_{—}005028.4 (protein) and NM_{—}005037.5 (nucleic acid).

Sequence information related to PPARγ (isoform 2, variant 2) is accessible in public databases by GenBank Accession numbers NP_{—}056953.2 (protein) and NM_{—}015869.4 (nucleic acid).

SEQ ID NO: 19 is the human wild type amino acid sequence corresponding to PPARγ (isoform 1, variant 1) (residues 1-477):

| | |
|---|---|
| 1 | MTMVDTEMPF WPTNFGISSV DLSVMEDHSH SFDIKPFTTV |
| | DFSSISTPHY EDIPFTRTDP |
| 61 | VVADYKYDLK LQEYQSAIKV EPASPPYYSE KTQLYNKPHE |
| | EPSNSLMAIE CRVCGDKASG |
| 121 | FHYGVHACEG CKGFFRRTIR LKLIYDRCDL NCRIHKKSRN |
| | KCQYCRFQKC LAVGMSHNAI |
| 181 | RFGRMPQAEK EKLLAEISSD IDQLNPESAD LRALAKHLYD |
| | SYIKSFPLTK AKARAILTGK |
| 241 | TTDKSPFVIY DMNSLMMGED KIKFKHITPL QEQSKEVAIR |
| | IFQGCQFRSV EAVQEITEYA |
| 301 | KSIPGFVNLD LNDQVTLLKY GVHEIIYTML ASLMNKDGVL |
| | ISEGQGFMTR EFLKSLRKPF |
| 361 | GDFMEPKFEF AVKFNALELD DSDLAIFIAV IILSGDRPGL |
| | LNVKPIEDIQ DNLLQALELQ |
| 421 | LKLNHPESSQ LFAKLLQKMT DLRQIVTEHV QLLQVIKKTE |
| | TDMSLHPLLQ EIYKDLY |

SEQ ID NO: 20 is the human wild type nucleotide sequence corresponding to PPARγ (isoform 1, variant 1) (nucleotides 1-1892), wherein the underscored bolded “ATG” denotes the beginning of the open reading frame:

| | |
|---|---|
| 1 | ggcgcccgcg cccgcccccg cgccgggccc ggctcggccc |
| | gacccggctc cgccgcgggc |
| 61 | aggcggggcc cagcgcactc ggagcccgag cccgagccgc |
| | agccgccgcc tggggcgctt |
| 121 | gggtcggcct cgaggacacc ggagaggggc gccacgccgc |
| | cgtggccgca gatttgaaag |
| 181 | aagccaacac taaaccacaa atatacaaca aggccatttt |
| | ctcaaacgag agtcagcctt |
| 241 | taacgaaatg accatggttg acacagagat gccattctgg |
| | cccaccaact ttgggatcag |
| 301 | ctccgtggat ctctccgtaa tggaagacca ctcccactcc |
| | tttgatatca agcccttcac |
| 361 | tactgttgac ttctccagca tttctactcc acattacgaa |
| | gacattccat tcacaagaac |
| 421 | agatccagtg gttgcagatt acaagtatga cctgaaactt |
| | caagagtacc aaagtgcaat |
| 481 | caaagtggag cctgcatctc caccttatta ttctgagaag |
| | actcagctct acaataagcc |
| 541 | tcatgaagag ccttccaact ccctcatggc aattgaatgt |
| | cgtgtctgtg gagataaagc |
| 601 | ttctggattt cactatggag ttcatgcttg tgaaggatgc |
| | aagggtttct tccggagaac |
| 661 | aatcagattg aagcttatct atgacagatg tgatcttaac |
| | tgtcggatcc acaaaaaaag |
| 721 | tagaaataaa tgtcagtact gtcggtttca gaaatgcctt |
| | gcagtgggga tgtctcataa |
| 781 | tgccatcagg tttgggcgga tgccacaggc cgagaaggag |
| | aagctgttgg cggagatctc |
| 841 | cagtgatatc gaccagctga atccagagtc cgctgacctc |
| | cgggccctgg caaaacattt |
| 901 | gtatgactca tacataaagt ccttcccgct gaccaaagca |
| | aaggcgaggg cgatcttgac |
| 961 | aggaaagaca acagacaaat caccattcgt tatctatgac |
| | atgaattcct taatgatggg |
| 1021 | agaagataaa atcaagttca aacacatcac ccccctgcag |
| | gagcagagca aagaggtggc |
| 1081 | catccgcatc tttcagggct gccagtttcg ctccgtggag |
| | gctgtgcagg agatcacaga |
| 1141 | gtatgccaaa agcattcctg gttttgtaaa tcttgacttg |
| | aacgaccaag taactctcct |
| 1201 | caaatatgga gtccacgaga tcatttacac aatgctggcc |
| | tccttgatga ataaagatgg |
| 1261 | ggttctcata tccgagggcc aaggcttcat gacaagggag |
| | tttctaaaga gcctgcgaaa |
| 1321 | gccttttggt gactttatgg agcccaagtt tgagtttgct |
| | gtgaagttca atgcactgga |
| 1381 | attagatgac agcgacttgg caatatttat tgctgtcatt |
| | attctcagtg gagaccgccc |
| 1441 | aggtttgctg aatgtgaagc ccattgaaga cattcaagac |
| | aacctgctac aagccctgga |
| 1501 | gctccagctg aagctgaacc accctgagtc ctcacagctg |
| | tttgccaagc tgctccagaa |
| 1561 | aatgacagac ctcagacaga ttgtcacgga acacgtgcag |
| | ctactgcagg tgatcaagaa |
| 1621 | gacggagaca gacatgagtc ttcacccgct cctgcaggag |
| | atctacaagg acttgtacta |
| 1681 | gcagagagtc ctgagccact gccaacattt cccttcttcc |
| | agttgcacta ttctgaggga |
| 1741 | aaatctgaca cctaagaaat ttactgtgaa aaagcatttt |
| | aaaaagaaaa ggttttagaa |
| 1801 | tatgatctat tttatgcata ttgtttataa agacacattt |
| | acaatttact tttaatatta |
| 1861 | aaaattacca tattatgaaa ttgctgatag ta |

For example, the polypeptide sequence of human GRHL3 (isoform 1) is depicted in SEQ ID NO: 21. The nucleotide sequence of human GRHL3 (isoform 1) is shown in SEQ ID NO: 22. Sequence information related to GRHL3 (isoform 1) is accessible in public databases by GenBank Accession numbers NP_{—}067003.2 (protein) and NM_{—}021180.3 (nucleic acid).

Sequence information related to GRHL3 (isoform 2) is accessible in public databases by GenBank Accession numbers NP_{—}937816.1 (protein) and NM_{—}198173.2 (nucleic acid).

Sequence information related to GRHL3 (isoform 3) is accessible in public databases by GenBank Accession numbers NP_{—}937817.3 (protein) and NM_{—}198174.2 (nucleic acid).

Sequence information related to GRHL3 (isoform 4) is accessible in public databases by GenBank Accession numbers NP_{—}1181939.1 (protein) and NM_{—}1195010.1 (nucleic acid).

SEQ ID NO: 21 is the human wild type amino acid sequence corresponding to GRHL3 (isoform 1) (residues 1-607):

| | |
|---|---|
| 1 | MWMNSILPIF LFRSVRLLKN DPVNLQKFSY TSEDEAWKTY |
| | LENPLTAATK AMMRVNGDDD |
| 61 | SVAALSFLYD YYMGPKEKRI LSSSTGGRND QGKRYYHGME |
| | YETDLTPLES PTHLMKFLTE |
| 121 | NVSGTPEYPD LLKKNNLMSL EGALPTPGKA APLPAGPSKL |
| | EAGSVDSYLL PTTDMYDNGS |
| 181 | LNSLFESIHG VPPTQRWQPD STFKDDPQES MLFPDILKTS |
| | PEPPCPEDYP SLKSDFEYTL |
| 241 | GSPKAIHIKS GESPMAYLNK GQFYPVTLRT PAGGKGLALS |
| | SNKVKSVVMV VFDNEKVPVE |
| 301 | QLRFWKHWHS RQPTAKQRVI DVADCKENFN TVEHIEEVAY |
| | NALSFVWNVN EEAKVFIGVN |
| 361 | CLSTDFSSQK GVKGVPLNLQ IDTYDCGLGT ERLVHRAVCQ |
| | IKIFCDKGAE RKMRDDERKQ |
| 421 | FRRKVKCPDS SNSGVKGCLL SGFRGNETTY LRPETDLETP |
| | PVLFIPNVHF SSLQRSGGAA |
| 481 | PSAGPSSSNR LPLKRTCSPF TEEFEPLPSK QAKEGDLQRV |
| | LLYVRRETEE VFDALMLKTP |
| 541 | DLKGLRNAIS EKYGFPEENI YKVYKKCKRG ILVNMDNNII |
| | QHYSNHVAFL LDMGELDGKI |
| 601 | QIILKEL |

SEQ ID NO: 22 is the human wild type nucleotide sequence corresponding to GRHL3 (isoform 1) (nucleotides 1-2710), wherein the underscored bolded “ATG” denotes the beginning of the open reading frame:

| | |
|---|---|
| 1 | aggagatgtg ccaaactgtt aagagtggtt atttctgagc agaagaatgt ggatgaattc |
| 61 | cattcttcct atttttcttt tcaggtctgt gcggctgcta aagaacgacc cagtcaactt |
| 121 | gcagaaattc tcttacacta gtgaggatga ggcctggaag acgtacctag aaaacccgtt |
| 181 | gacagctgcc acaaaggcca tgatgagagt caatggagat gatgacagtg ttgcggcctt |
| 241 | gagcttcctc tatgattact acatgggtcc caaggagaag cggatattgt cctccagcac |
| 301 | tgggggcagg aatgaccaag gaaagaggta ctaccatggc atggaatatg agacggacct |
| 361 | cactcccctt gaaagcccca cacacctcat gaaattcctg acagagaacg tgtctggaac |
| 421 | cccagagtac ccagatttgc tcaagaagaa taacctgatg agcttggagg gggccttgcc |
| 481 | cacccctggc aaggcagctc ccctccctgc aggccccagc aagctggagg ccggctctgt |
| 541 | ggacagctac ctgttaccca ccactgatat gtatgataat ggctccctca actccttgtt |
| 601 | tgagagcatt catggggtgc cgcccacaca gcgctggcag ccagacagca ccttcaaaga |
| 661 | tgacccacag gagtcgatgc tcttcccaga tatcctgaaa acctccccgg aacccccatg |
| 721 | tccagaggac taccccagcc tcaaaagtga ctttgaatac accctgggct cccccaaagc |
| 781 | catccacatc aagtcaggcg agtcacccat ggcctacctc aacaaaggcc agttctaccc |
| 841 | cgtcaccctg cggaccccag caggtggcaa aggccttgcc ttgtcctcca acaaagtcaa |
| 901 | gagtgtggtg atggttgtct tcgacaatga gaaggtccca gtagagcagc tgcgcttctg |
| 961 | gaagcactgg cattcccggc aacccactgc caagcagcgg gtcattgacg tggctgactg |
| 1021 | caaagaaaac ttcaacactg tggagcacat tgaggaggtg gcctataatg cactgtcctt |
| 1081 | tgtgtggaac gtgaatgaag aggccaaggt gttcatcggc gtaaactgtc tgagcacaga |
| 1141 | cttttcctca caaaaggggg tgaagggtgt ccccctgaac ctgcagattg acacctatga |
| 1201 | ctgtggcttg ggcactgagc gcctggtaca ccgtgctgtc tgccagatca agatcttctg |
| 1261 | tgacaaggga gctgagagga agatgcgcga tgacgagcgg aagcagttcc ggaggaaggt |
| 1321 | caagtgccct gactccagca acagtggcgt caagggctgc ctgctgtcgg gcttcagggg |
| 1381 | caatgagacg acctaccttc ggccagagac tgacctggag acgccacccg tgctgttcat |
| 1441 | ccccaatgtg cacttctcca gcctgcagcg ctctggaggg gcagccccct cggcaggacc |
| 1501 | cagcagctcc aacaggctgc ctctgaagcg tacctgctcg cccttcactg aggagtttga |
| 1561 | gcctctgccc tccaagcagg ccaaggaagg cgaccttcag agagttctgc tgtatgtgcg |
| 1621 | gagggagact gaggaggtgt ttgacgcgct catgttgaag accccagacc tgaaggggct |
| 1681 | gaggaatgcg atctctgaga agtatgggtt ccctgaagag aacatttaca aagtctacaa |
| 1741 | gaaatgcaag cgaggaatct tagtcaacat ggacaacaac atcattcagc attacagcaa |
| 1801 | ccacgtcgcc ttcctgctgg acatggggga gctggacggc aaaattcaga tcatccttaa |
| 1861 | ggagctgtaa ggcctctcga gcatccaaac cctcacgacc tgcaaggggc cagcagggac |
| 1921 | gtggccccac gccacacaca acctctccac atgcctcagc gctgttactt gaatgccttc |
| 1981 | cctgagggaa gaggcccttg agtcacagac ccacagacgt cagggccagg gagagaccta |
| 2041 | gggggtcccc tggcctggat ccccatggta tgcttgaatc tgctccctga acttcctgcc |
| 2101 | agtgcctccc cgtaccccaa aacaatgtca ccatggttac cacctaccca gaagactgtt |
| 2161 | ccctcctccc aagacccttg tctgcagtgg tgctcctgca ggctgcccgt taagatggtg |
| 2221 | gcggcacacg ctccctcccg cagcaccacg ccagctggtg cggcccccac tctctgtctt |
| 2281 | ccttcaactt cagacaaagg atttctcaac ctttggtcag ttaacttgaa aactcttgat |
| 2341 | tttcagtgca aatgactttt aaaagacact atattggagt ctctttctca gacttcctca |
| 2401 | gcgcaggatg taaatagcac taacgatcga ctggaacaaa gtgaccgctg tgtaaaacta |
| 2461 | ctgccttgcc actcactgtt gtatacattt cttatttacg attttcattt gttatatata |
| 2521 | tatataaata tactgtatat atatgcaaca ttttatattt ttcatggata tgtttttatc |
| 2581 | atttcaaaaa atgtgtattt cacatttctt ggactttttt tagctgttat tcagtgatgc |
| 2641 | attttgtata ctcacgtggt atttagtaat aaaaatctat ctatgtatta cgtcacatta |
| 2701 | aaaaaaaaaa |

For example, the polypeptide sequence of human ELF3 (transcript variant 1) is depicted in SEQ ID NO: 23. The nucleotide sequence of human ELF3 (transcript variant 1) is shown in SEQ ID NO: 24. Sequence information related to ELF3 (transcript variant 1) is accessible in public databases by GenBank Accession numbers NP_{—}004424.3 (protein) and NM_{—}004433.4 (nucleic acid).

Sequence information related to ELF3 (transcript variant 2) is accessible in public databases by GenBank Accession numbers NP_{—}1107781.1 (protein) and NM_{—}1114309.1 (nucleic acid).

SEQ ID NO: 23 is the human wild type amino acid sequence corresponding to ELF3 (transcript variant 1) (residues 1-371):

| | |
|---|---|
| 1 | MAATCEISNI FSNYFSAMYS SEDSTLASVP PAATFGADDL |
| | VLTLSNPQMS LEGTEKASWL |
| 61 | GEQPQFWSKT QVLDWISYQV EKNKYDASAI DFSRCDMDGA |
| | TLCNCALEEL RLVFGPLGDQ |
| 121 | LHAQLRDLTS SSSDELSWII ELLEKDGMAF QEALDPGPFD |
| | QGSPFAQELL DDGQQASPYH |
| 181 | PGSCGAGAPS PGSSDVSTAG TGASRSSHSS DSGGSDVDLD |
| | PTDGKLFPSD GFRDCKKGDP |
| 241 | KHGKRKRGRP RKLSKEYWDC LEGKKSKHAP RGTHLWEFIR |
| | DILIHPELNE GLMKWENRHE |
| 301 | GVFKFLRSEA VAQLWGQKKK NSNMTYEKLS RAMRYYYKRE |
| | ILERVDGRRL VYKFGKNSSG |
| 361 | WKEEEVLQSR N |

SEQ ID NO: 24 is the human wild type nucleotide sequence corresponding to ELF3 (transcript variant 1) (nucleotides 1-3149), wherein the underscored bolded “ATG” denotes the beginning of the open reading frame:

| | |
|---|---|
| 1 | ctgagctcag ggaggagctc cctccaggct ctatttagag ccgggtaggg gagcgcagcg |
| 61 | gccagatacc tcagcgctac ctggcggaac tggatttctc tcccgcctgc cggcctgcct |
| 121 | gccacagccg gactccgcca ctccggtagc ctcatggctg caacctgtga gattagcaac |
| 181 | atttttagca actacttcag tgcgatgtac agctcggagg actccaccct ggcctctgtt |
| 241 | ccccctgctg ccacctttgg ggccgatgac ttggtactga ccctgagcaa cccccagatg |
| 301 | tcattggagg gtacagagaa ggccagctgg ttgggggaac agccccagtt ctggtcgaag |
| 361 | acgcaggttc tggactggat cagctaccaa gtggagaaga acaagtacga cgcaagcgcc |
| 421 | attgacttct cacgatgtga catggatggc gccaccctct gcaattgtgc ccttgaggag |
| 481 | ctgcgtctgg tctttgggcc tctgggggac caactccatg cccagctgcg agacctcact |
| 541 | tccagctctt ctgatgagct cagttggatc attgagctgc tggagaagga tggcatggcc |
| 601 | ttccaggagg ccctagaccc agggcccttt gaccagggca gcccctttgc ccaggagctg |
| 661 | ctggacgacg gtcagcaagc cagcccctac caccccggca gctgtggcgc aggagccccc |
| 721 | tcccctggca gctctgacgt ctccaccgca gggactggtg cttctcggag ctcccactcc |
| 781 | tcagactccg gtggaagtga cgtggacctg gatcccactg atggcaagct cttccccagc |
| 841 | gatggttttc gtgactgcaa gaagggggat cccaagcacg ggaagcggaa acgaggccgg |
| 901 | ccccgaaagc tgagcaaaga gtactgggac tgtctcgagg gcaagaagag caagcacgcg |
| 961 | cccagaggca cccacctgtg ggagttcatc cgggacatcc tcatccaccc ggagctcaac |
| 1021 | gagggcctca tgaagtggga gaatcggcat gaaggcgtct tcaagttcct gcgctccgag |
| 1081 | gctgtggccc aactatgggg ccaaaagaaa aagaacagca acatgaccta cgagaagctg |
| 1141 | agccgggcca tgaggtacta ctacaaacgg gagatcctgg aacgggtgga tggccggcga |
| 1201 | ctcgtctaca agtttggcaa aaactcaagc ggctggaagg aggaagaggt tctccagagt |
| 1261 | cggaactgag ggttggaact atacccggga ccaaactcac ggaccactcg aggcctgcaa |
| 1321 | accttcctgg gaggacaggc aggccagatg gcccctccac tggggaatgc tcccagctgt |
| 1381 | gctgtggaga gaagctgatg ttttggtgta ttgtcagcca tcgtcctggg actcggagac |
| 1441 | tatggcctcg cctccccacc ctcctcttgg aattacaagc cctggggttt gaagctgact |
| 1501 | ttatagctgc aagtgtatct ccttttatct ggtgcctcct caaacccagt ctcagacact |
| 1561 | aaatgcagac aacaccttcc tcctgcagac acctggactg agccaaggag gcctggggag |
| 1621 | gccctagggg agcaccgtga tggagaggac agagcagggg ctccagcacc ttctttctgg |
| 1681 | actggcgttc acctccctgc tcagtgcttg ggctccacgg gcaggggtca gagcactccc |
| 1741 | taatttatgt gctatataaa tatgtcagat gtacatagag atctattttt tctaaaacat |
| 1801 | tcccctcccc actcctctcc cacagagtgc tggactgttc caggccctcc agtgggctga |
| 1861 | tgctgggacc cttaggatgg ggctcccagc tcctttctcc tgtgaatgga ggcagagacc |
| 1921 | tccaataaag tgccttctgg gctttttcta acctttgtct tagctacctg tgtactgaaa |
| 1981 | tttgggcctt tggatcgaat atggtcaaga ggttggaggg gaggaaaatg aaggtctacc |
| 2041 | aggctgaggg tgagggcaaa ggctgacgaa gaggggagtt acagatttcc tgtagcaggt |
| 2101 | gtgggcttac agacacatgg actgggctgg gaggcgagca aaggaagcag ctgagactgt |
| 2161 | tggagaacgc ttacaagact tcatgcaagc aaggacatga actcagaaca ctgaggtcag |
| 2221 | aagcatcctg ctgtcatgac accgctcgag tgaccttgac cttgaccaag tctgtcctgt |
| 2281 | ttaggactga tttttcctat taggctaggg tttggacctg atgttctcaa gatgtctaga |
| 2341 | attgcatggc tggccttgtg gaatagatgg ttttgcattc cagccaagtg tgctgtaaac |
| 2401 | tgtatatctg taatatgaat cccagctttt gagtctgaca aaatcagagt taggatcttg |
| 2461 | taaaggaaaa aaaaaaaaaa acaaaacaaa atggagatga gtacttgctg agaaagaatg |
| 2521 | agggaaggag ttggcatttg ttgaaagtgt agtctttttc tctttttttt ttaattgcaa |
| 2581 | cttttacttt agatttagga ggtcgtgcgc aggtttgtta catgggtata ttgtgtgatg |
| 2641 | ctgagcttgg gatgcgaatg atcctgtcac ccaggtagtg agtatagcac ccagtgaaac |
| 2701 | tgtagtctca tgccaggcac tgtgctagcc cactctggct catttaatcc tctcctaaga |
| 2761 | agagaggaga cacagcgtcc ccatttgaca gatgcagaaa gaggttccac aggtgtgcct |
| 2821 | tgattctgtc ctaaaaccgt ttcccggaag cttttcctgg tgtgggcgct tctaacctaa |
| 2881 | tcctcaatcg attccagaac tattactctg tttccacagt gatactgtgt ctaggtttta |
| 2941 | gggaggacag ttcattgatg ttacttaaga atgctttcca ggtggaaagt tccttaagtt |
| 3001 | tgaggcttca aattccatac agcacattaa aatcccattc atgagtttga aatactgctc |
| 3061 | tgttgtcttg gaaataccaa tcagattgtt ggctgaagtg atgtggataa agaagggatc |
| 3121 | ttagaaaaac taaaaaaaaa aaaaaaaaa |

For example, the polypeptide sequence of human EHF (isoform 1) is depicted in SEQ ID NO: 25. The nucleotide sequence of human EHF (isoform 1) is shown in SEQ ID NO: 26. Sequence information related to EHF (isoform 1) is accessible in public databases by GenBank Accession numbers NP_{—}1193545.1 (protein) and NM_{—}1206616.1 (nucleic acid).

Sequence information related to EHF (isoform 2) is accessible in public databases by GenBank Accession numbers NP_{—}036285.2 (protein) and NM_{—}012153.5 (nucleic acid).

Sequence information related to EHF (isoform 3) is accessible in public databases by GenBank Accession numbers NP_{—}1193544.1 (protein) and NM_{—}1206615.1 (nucleic acid).

SEQ ID NO: 25 is the human wild type amino acid sequence corresponding to EHF (isoform 1) (residues 1-322):

| | |
|---|---|
| 1 | MGLPERRGLV LLLSLAEILF KIMILEGGGV MNLNPGNNLL |
| | HQPPAWTDSY STCNVSSGFF |
| 61 | GGQWHEIHPQ YWTKYQVWEW LQHLLDTNQL DANCIPFQEF |
| | DINGEHLCSM SLQEFTRAAG |
| 121 | TAGQLLYSNL QHLKWNGQCS SDLFQSTHNV IVKTEQTEPS |
| | IMNTWKDENY LYDTNYGSTV |
| 181 | DLLDSKTFCR AQISMTTTSH LPVAESPDMK KEQDPPAKCH |
| | TKKHNPRGTH LWEFIRDILL |
| 241 | NPDKNPGLIK WEDRSEGVFR FLKSEAVAQL WGKKKNNSSM |
| | TYEKLSRAMR YYYKREILER |
| 301 | VDGRRLVYKF GKNARGWREN EN |

SEQ ID NO: 26 is the human wild type nucleotide sequence corresponding to EHF (isoform 1) (nucleotides 1-5467), wherein the underscored bolded “ATG” denotes the beginning of the open reading frame:

| | |
|---|---|
| 1 | aacccactgc tttattctgc cctgagtgga gattggtttt ggctcaggct gctttgtgaa |
| 61 | actcagaagc attatcctct ctgccaactc cacgtcctag tcagagtttt ctgtgaaggc |
| 121 | aagggcatgg ggttgccgga gagaagagga ttggtcctgc ttttaagcct agctgaaatt |
| 181 | cttttcaaga tcatgattct ggaaggaggt ggtgtaatga atctcaaccc cggcaacaac |
| 241 | ctccttcacc agccgccagc ctggacagac agctactcca cgtgcaatgt ttccagtggg |
| 301 | ttttttggag gccagtggca tgaaattcat cctcagtact ggaccaagta ccaggtgtgg |
| 361 | gagtggctcc agcacctcct ggacaccaac cagctggatg ccaattgtat ccctttccaa |
| 421 | gagttcgaca tcaacggcga gcacctctgc agcatgagtt tgcaggagtt cacccgggcg |
| 481 | gcagggacgg cggggcagct cctctacagc aacttgcagc atctgaagtg gaacggccag |
| 541 | tgcagtagtg acctgttcca gtccacacac aatgtcattg tcaagactga acaaactgag |
| 601 | ccttccatca tgaacacctg gaaagacgag aactatttat atgacaccaa ctatggtagc |
| 661 | acagtagatt tgttggacag caaaactttc tgccgggctc agatctccat gacaaccacc |
| 721 | agtcaccttc ctgttgcaga gtcacctgat atgaaaaagg agcaagaccc ccctgccaag |
| 781 | tgccacacca aaaagcacaa cccgagaggg actcacttat gggaattcat ccgcgacatc |
| 841 | ctcttgaacc cagacaagaa cccaggatta ataaaatggg aagaccgatc tgagggcgtc |
| 901 | ttcaggttct tgaaatcaga ggcagtggct cagctatggg gtaaaaagaa gaacaacagc |
| 961 | agcatgacct atgaaaagct cagccgagct atgagatatt actacaaaag agaaattctg |
| 1021 | gagcgtgtgg atggacgaag actggtatat aaatttggga agaatgcccg aggatggaga |
| 1081 | gaaaatgaaa actgaagctg ccaatacttt ggacacaaac caaaacacac accaaataat |
| 1141 | cagaaacaaa gaactcctgg acgtaaatat ttcaaagact acttttctct gatatttatg |
| 1201 | taccatgagg ggaacaagaa actacttcta acgggaagaa gaaacactac agtcgattaa |
| 1261 | aaaaattatt ttgttacttc gaagtatgtc ctatatgggg aaaaaacgta cacagttttc |
| 1321 | tgtgaaatat gatgctgtat gtggttgtga ttttttttca cctctattgt gaattctttt |
| 1381 | tcactgcaag agtaacagga tttgtagcct tgtgcttctt gctaagagaa agaaaaacaa |
| 1441 | aatcagaggg cattaaatgt tttgtatgtg acatgattta gaaaaaggtg atgcatcctc |
| 1501 | ctcacataag catccatatg gcttcgtcaa gggaggtgaa cattgttgct gagttaaatt |
| 1561 | ccagggtctc agatggttag gacaaagtgg atggatgccg ggaagtttaa cctgagcctt |
| 1621 | aggatccaat gagtggagaa tggggacttc caaaacccaa ggttggctat aatctctgca |
| 1681 | taaccacatg acttggaatg cttaaatcag caagaagaat aatggtgggg tctttatact |
| 1741 | cattcaggaa tggtttatct gatgccaggg ctgtcttcct ttctcccctt tggatggttg |
| 1801 | gtgaaatact ttaattgccc tgtctgctca cttctagcta tttaagagag aacccagctt |
| 1861 | ggttcttttt tgctccaagt gcttaaaaat aagttggaaa aaggagacgg tggtgtggaa |
| 1921 | atggctgaag agtttgctct tgtatcccta tagtccaagg tttctcaatc tgcacaattg |
| 1981 | acatttttgg ccggagtgtt ctttgtggtg agggctttcc tgtgcattgt aagatgttca |
| 2041 | gcagtatcca ctcatggtct ctaaccactt gacaccagaa accccccagc tgtgataacg |
| 2101 | caaaatgtct ctagacatca ccaaatgttc cctgggggtg gcaaatttgc ccttgattga |
| 2161 | gaaccaccag tttagctagt caatatgagg atggtggttt attctcagaa gaaaaagata |
| 2221 | tgtaaggtct tttagctcct tagagtgaag caaaagcaag acttcaacct caacctatct |
| 2281 | ttatgtttta aatgttaggg acaataagtt gaaatagcta gaggagcttc ttttcagaac |
| 2341 | cccagatgag agccaatgtc agataaagta agcatagtaa tgtagcagga actacaatag |
| 2401 | aagacatttt cactggaatt acaaagcaga attaaaatta tattgtagaa ggaaacacca |
| 2461 | agaaaagaat ttccagggaa aatcctcttt gcaggtatta attcttataa ttttttgtct |
| 2521 | tttggattat ctgtttactg tctcatctga actgatccca ggtgaacggt ttattgccta |
| 2581 | gatttgtact cagaggaatt ttttttgttt tgttttgtct tttaagaaag gaaagaaagg |
| 2641 | atgaaaaaaa taaacagaaa actcagctca ggcacaattg tcaccaagga gttaaaagct |
| 2701 | tcttcttcaa tagaggaatt gttctggggg tcctggagac ttaccattga gccatgcaat |
| 2761 | ctgggaagca caggaataag tagacacttt gaaaatggat ttgaatgttc tcatcccttt |
| 2821 | tgcagctttt ctttttggct ctctcatgtc cttggcttgc tcctctattc tacctctctt |
| 2881 | tctccagcaa taatatgcaa atgaagacat gtatccataa gaaggagtgc tcttcatcaa |
| 2941 | ctaatagagc acctaccaca gtgtcatacc tggtagaggt gagcaattca tattcaaagg |
| 3001 | ttgcaaagtg tttgtaatat attcatgagg ctggaagtaa gaagaattaa aaatttgtcc |
| 3061 | taattacaat gagaaccatt ctaggtagtg atcttggagc acacatgaat aactttctga |
| 3121 | aggtgcaacc aaatccattt ttatttctgc ctggcttggt cacttctgta aaggtttaac |
| 3181 | ttagtgttgt caagtaacag ttactgaaag agctgagaaa aagaacaatg aacagcaacg |
| 3241 | atcttgactg tgcaactcag acattcctgc agaaaagaca tatgttgctt tacaagaagg |
| 3301 | ccaaagaact atggggcctt cccagcattt gactgttcat tgcatagaat gaattaaata |
| 3361 | tccagttact tgaatgggta taacgcatga atatttgtgt gtctgtgtgt gtgtctgagt |
| 3421 | tgtgtgattt tattaggggc atctgccaat tctctcactg tggttccttc tctgactttg |
| 3481 | cctgttcatc atctaaggag gctagatcct tcgctgactt caccattcct caaacctgta |
| 3541 | agtttctcac ttcttccaaa ttggctttgg ctctttctgc aacctttcca ttcaagagca |
| 3601 | atctttgcta aggagtaagt gaatgtgaag agtaccaact acaacaattc tacagataat |
| 3661 | tagtggattg tgttgtttgt tgagagtgaa ggtttcttgg catctggtgc ctgattaagg |
| 3721 | cttgagtatt aagttctcag catatctctc tattgtcttg acttgagttt gctgcatttt |
| 3781 | ctatgtgctg ttcgtgactt ggagaactta aagtaatcga gctatgccaa cttggggtgg |
| 3841 | taacagagta cttcccacca cagtgttgaa agggagagca aagtcttatg gataaaccct |
| 3901 | cctttctttt ggggacacat ggctctcact tgagaagctc acctgtgctg aatgtccaca |
| 3961 | tggtcactaa acatgttatc cttaaacccc ccgtatgcct gagttgaaag ggctctctct |
| 4021 | tattaggttt tcatgggaac atgaggcagc aaatctattg ctaagacttt accaggctca |
| 4081 | aatcatctga ggctgataga tatttgactt ggtaagactt aagtaaggct ctggctccca |
| 4141 | ggggcataag caacagtttc ttgaatgtgc catctgagaa gggagaccca ggttgtgagt |
| 4201 | tttcctttga acacattggt cttttctcaa agttcctgcc ttgctagact gttagctctt |
| 4261 | tgaggacagg gactatgtct tatcaatcac tattattttc ctgttaccta gcatgggaca |
| 4321 | agtacacaac acatatttgt tcaatgaatg aatgaatgtc ttctaaaaga ctcctctgat |
| 4381 | tgggagacca tatctataat tgggatgtga atcatttctt cagtggaata agagcacaac |
| 4441 | ggcacaacct tcaaggacat attatctact atgaacattt tactgtgaga ctctttattt |
| 4501 | tgccttctac ttgcgctgaa atgaaaccaa aacaggccgt tgggttccac aagtcaatat |
| 4561 | atgttggatg aggattctgt tgccttattg ggaactgtga gacttatctg gtatgagaag |
| 4621 | ccagtaataa acctttgacc tgttttaacc aatgaagatt atgaatatgt taatatgatg |
| 4681 | taaattgcta tttaagtgta aagcagttct aagttttagt atttggggga ttggttttta |
| 4741 | ttattttttt cctttttgaa aaatactgag ggatcttttg ataaagttag taatgcatgt |
| 4801 | tagattttag ttttgcaagc atgttgtttt tcaaatatat caagtataga aaaaggtaaa |
| 4861 | acagttaaga aggaaggcaa ttatattatt cttctgtagt taagcaaaca cttgttgagt |
| 4921 | gcctgctatg tgcacggcat gggcccatat gtgtgaggag cttgtctaat tatgtaggaa |
| 4981 | gcaatagatc tcggtagtta cgtattgggc agatacttac tgtatgaatg aaagaacatc |
| 5041 | acagtaatca caatatcaga gctgaattat cctcagtgta gcttcttgga attcagtttc |
| 5101 | tggaactaga gatagagcat ttattaaaaa aaactcctgt tgagactgtg tcttatgaac |
| 5161 | ctctgaaacg tacaagcctt cacaagttta actaaattgg gattaatctt tctgtagtta |
| 5221 | tctgcataat tcttgttttt ctttccatct ggctcctggg ttgacaattt gtggaaacaa |
| 5281 | ctctattgct actatttaaa aaaaatcaga aatctttccc tttaagctat gttaaattca |
| 5341 | aactattcct gctattcctg ttttgtcaaa gaattatatt tttcaaaata tgtttatttg |
| 5401 | tttgatgggt cccaggaaac actaataaaa accacagaga ccagcctgga aaaaaaaaaa |
| 5461 | aaaaaaa |

A reprogramming factor molecule or a master regulatory molecule can also encompass ortholog genes, which are genes conserved among different biological species such as humans, dogs, cats, mice, and rats, that encode proteins (for example, homologs (including splice variants), mutants, and derivatives) having biologically equivalent functions as the human-derived protein. Orthologs of a reprogramming factor molecule or a master regulatory molecule include any mammalian ortholog inclusive of the ortholog in humans and other primates, experimental mammals (such as mice, rats, hamsters and guinea pigs), mammals of commercial significance (such as horses, cows, camels, pigs and sheep), and also companion mammals (such as domestic animals, e.g., rabbits, ferrets, dogs, and cats).

In one embodiment of the present invention, the gene encoding a protein of interest (for example for example, Oct4, Sox2, Klf4, c-Myc, NKX3.1, AR, FOXA1, FOXA2, KLF5, Pparγ, Grhl3, Elf3, Ehf, and the like), can be cloned from either a genomic library or a cDNA according to standard protocols familiar to one skilled in the art (J. Sambrook et al., 1989, Molecular Cloning, A Laboratory Manual, Cold Spring Harbor Press, Plainview, N.Y.; F. M. Ausubel et al., 1989, Current Protocols in Molecular Biology, John Wiley & Sons, New York, N.Y.). A cDNA, for example, encoding Oct4, Sox2, Klf4, c-Myc, NKX3.1, AR, FOXA1, FOXA2, KLF5, Pparγ, Grhl3, Elf3, or Ehf, can be obtained by isolating total mRNA from a suitable cell line. Double stranded cDNAs can be prepared from the total mRNA using methods known in the art, and subsequently can be inserted into a suitable plasmid or vector. Genes can also be cloned using PCR techniques well established in the art. In one embodiment, a gene encoding Oct4, Sox2, Klf4, c-Myc, NKX3.1, AR, FOXA1, FOXA2, KLF5, Pparγ, Grhl3, Elf3, or Ehf, can be cloned via PCR in accordance with the nucleotide sequence information provided by Genbank. In a further embodiment, a DNA vector containing Oct4, Sox2, Klf4, c-Myc, NKX3.1, AR, FOXA1, FOXA2, KLF5, Pparγ, Grhl3, Elf3, or Ehf, can act as a template in PCR reactions wherein oligonucleotide primers designed to amplify a region of interest can be used, so as to obtain an isolated DNA fragment encompassing that region.

An expression vector of the current invention can include nucleotide sequences that encode either an Oct4, Sox2, Klf4, c-Myc, NKX3.1, AR, FOXA1, FOXA2, KLF5, Pparγ, Grhl3, Elf3, or Ehf protein linked to at least one sequence in a manner allowing expression of the nucleotide sequence in a host cell. Regulatory sequences are well known to those skilled in the art, and can be selected to direct the expression of a protein of interest (such as Oct4, Sox2, Klf4, c-Myc, NKX3.1, AR, FOXA1, FOXA2, KLF5, Pparγ, Grhl3, Elf3, or Ehf) in an appropriate host cell as described in Goeddel, Gene Expression Technology: Methods in Enzymology 185, Academic Press, San Diego, Calif. (1990). Non-limiting examples of regulatory sequences include: polyadenylation signals, promoters (such as CMV, ASV, SV40, or other viral promoters such as those derived from bovine papilloma, polyoma, and Adenovirus 2 viruses (Fiers, et al., 1973, Nature 273:113; Hager G L et al., Curr Opin Genet Dev, 2002, 12(2):137-41) enhancers, and other expression control elements.

One skilled in the art also understands that enhancer regions, which are those sequences found upstream or downstream of the promoter region in non-coding DNA regions, are also important in optimizing expression. If needed, origins of replication from viral sources can be employed, such as if a prokaryotic host is utilized for introduction of plasmid DNA. However, in eukaryotic organisms, chromosome integration is a common mechanism for DNA replication.

In one embodiment of the present invention, the gene encoding a protein of interest (such as Oct4, Sox2, Klf4, c-Myc, NKX3.1, AR, FOXA1, FOXA2, KLF5, Pparγ, Grhl3, Elf3, or Ehf) is controlled by an inducible promoter. For example, transcription of the gene encoding a protein of interest is reversibly controlled by the presence of an antibiotic, such as doxycycline. Inducible expression systems are well known in the art, and include but are not limited to, the Tet-On system, or the Tet-Off system (U.S. Pat. No. 5,464,758; U.S. Pat. No. 5,814,618; Bujard H. & Gossen M., 1992, PNAS 89(12):5547-51)

It is understood by those skilled in the art that for stable amplification and expression of a desired protein, a vector harboring DNA encoding a protein of interest (for example, Oct4, Sox2, Klf4, c-Myc, NKX3.1, AR, FOXA1, FOXA2, KLF5, Pparγ, Grhl3, Elf3, or Ehf) is stably integrated into the genome of eukaryotic cells (for example, mammalian cells, such as mouse embryonic fibroblasts, mouse dermal fibroblasts, or BJ normal human foreskin fibroblasts), resulting in the stable expression of transfected genes. The expression vector and method of introduction of the exogenous nucleic acid to the cell can be factors that contribute to a successful integration event. For example, an exogenous nucleic acid can be integrated into the genome of eukaryotic cells (such as a mammalian cell) for stable expression by using a retrovirus to introduce the exogenous nucleic acid into the cell. In another example, an exogenous nucleic acid sequence can be introduced into a cell by homologous recombination as disclosed in U.S. Pat. No. 5,641,670, the contents of which are herein incorporated by reference.

A gene that encodes a selectable marker (for example, resistance to antibiotics or drugs, such as ampicillin, G418, and hygromycin) can be introduced into host cells along with the gene of interest in order to identify and select clones that stably express a gene encoding a protein of interest. The gene encoding a selectable marker can be introduced into a host cell on the same plasmid as the gene of interest or can be introduces on a separate plasmid. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein (for example, Oct4, Sox2, Klf4, c-Myc, NKX3.1, AR, FOXA1, FOXA2, KLF5, Pparγ, Grhl3, Elf3, or Ehf)

Introduction of Reprogramming Factors into Fibroblasts

A eukaryotic expression vector can be introduced into cells in order to produce proteins (for example, Oct4, Sox2, Klf4, or c-Myc) encoded by nucleotide sequences of the vector. Cells (such as embryonic fibroblasts, mouse dermal fibroblasts, or BJ normal human foreskin fibroblasts) can harbor an expression vector (for example, one that contains a gene encoding Oct4, Sox2, Klf4, or c-Myc) via introducing the expression vector into an appropriate host cell via methods known in the art.

An exogenous nucleic acid can be introduced into a cell via a variety of techniques known in the art. For example, a retrovirus can be used to introduce a nucleotide sequence into cells (such as embryonic fibroblasts, mouse dermal fibroblasts, or BJ normal human foreskin fibroblasts). In one embodiment, the retrovirus is a Rebna retrovirus. Other viral vectors known in the art can be used to introduce a nucleotide sequence, including, but not limited to a lentivirus, a adenovirus, or a adeno-associated virus.

In one embodiment, a retrovirus can be used to introduce a nucleotide sequence into embryonic fibroblasts, dermal fibroblasts, or human foreskin fibroblasts, in order to produce proteins encoded by said nucleotide sequences (for example, Oct4, Sox2, Klf4, and c-Myc). For example, the Rebna retrovirus is used to introduce DNA into an embryonic fibroblast, or a dermal fibroblast, to confer high-level stable expression of reprogramming factors (for example, Oct4, Sox2, Klf4, and c-Myc). In other embodiments, lentivirus is used to introduce DNA into embryonic fibroblasts, dermal fibroblasts, or human foreskin fibroblasts, to confer high-level stable expression of reprogramming factors (for example, Oct4, Sox2, Klf4, and c-Myc). In further embodiments, lentivirus is used to introduce DNA into embryonic fibroblasts, dermal fibroblasts, or human foreskin fibroblasts to confer transient doxycycline-inducible expression of reprogramming factors (for example, Oct4, Sox2, Klf4, and c-Myc). The nucleic acid of interest can encode only a single protein (for example, Oct4, Sox2, Klf4, or c-Myc), or can encode for more than one proteins of interest (for example, combinations of Oct4, Sox2, Klf4, c-Myc). In one embodiment, doxycycline-inducible expression of reprogramming factors (for example, Oct4, Sox2, Klf4, and/or c-Myc) is used. Reprogramming factors include, but are not limited to, Oct4, Sox2, Klf4, c-Myc, nanog, Lin28, Esrrb, or Nr5a2.

A eukaryotic expression vector can be used to transfect cells in order to produce proteins (for example, Oct4, Sox2, Klf4, or c-Myc) encoded by nucleotide sequences of the vector. Mammalian cells (such as mouse embryonic fibroblasts, mouse dermal fibroblasts, or BJ normal human foreskin fibroblasts) can harbor an expression vector (for example, one that encodes a gene encoding Oct4, Sox2, Klf4, or c-Myc) via introducing the expression vector into an appropriate host cell via methods known in the art.

An exogenous nucleic acid can be introduced into a cell via a variety of techniques known in the art, such as lipofection, microinjection, calcium phosphate or calcium chloride precipitation, DEAE-dextrin-mediated transfection, or electroporation. Other methods used to transfect cells can also include calcium phosphate precipitation, modified calcium phosphate precipitation, polybrene precipitation, microinjection liposome fusion, and receptor-mediated gene delivery.

Cells to be genetically engineered can be primary and secondary cells, which can be obtained from various tissues and include cell types which can be maintained and propagated in culture. Vertebrate tissue can be obtained by methods known to one skilled in the art, such as dissection of an E13.5 mouse embryo. In one embodiment, tissue can be obtained from an E12.5, E13, E13.5, E14, or E14.5 mouse embryo. In another embodiment, dissection of a E13.5 mouse embryo can be used to obtain a source of embryonic fibroblast cells. In further embodiments, tissue can be obtained from a P0, P1, P2, or P3 mouse. For example, dissection of a P0 mouse can be used to obtain a source of mouse dermal fibroblasts. In another embodiment, human foreskins can be used to obtain a source of BJ normal human foreskin fibroblasts.

In certain embodiments, embryonic fibroblast cells or mouse dermal fibroblasts can be acquired from a mouse which has been genetically engineered. For example, embryonic fibroblasts or mouse dermal fibroblasts may be derived from mice with an Oct4-GFP knock-in genotype. In another embodiment, embryonic fibroblasts or mouse dermal fibroblasts may be derived from mice with a Nkx3.1-lacZ knock-in genotype. In further embodiments, embryonic fibroblasts or mouse dermal fibroblasts may be derived from mice with a doxycycline-regulated transgene encoding a protein, or proteins of interest (for example, Oct4, Sox2, Klf4, c-Myc, or a combination thereof). Embryonic fibroblasts or mouse dermal fibroblasts may also be derived from mice with other genetically engineered genomes including, but not limited to, Nanog-CreER^{T2};R26R-Tomato mice, CK5-CreER^{T2}; R26R-YFP mice, CK8-CreER^{T2}; R26R-YFP mice, or CK18-CreER^{T2}; R26R-YFP mice. In other embodiments, embryonic fibroblast cells or mouse dermal fibroblast cells can be acquired from a mouse which has a wild-type genome. In some embodiments, embryonic fibroblasts or mouse dermal fibroblasts may be derived from mice with a GATA6CreERT2; R26R-CAG-YFP genotype. In some embodiments, embryonic fibroblasts or mouse dermal fibroblasts may be derived from mice with a CK18CreERT2; R26R-Tomato genotype.

Cell Culturing of Eukaryotic Cells

Various culturing parameters can be used with respect to the host cell being cultured. Appropriate culture conditions for mammalian cells are well known in the art or can be determined by the skilled artisan (see, for example, Animal Cell Culture: A Practical Approach 2^{nd} Ed., Rickwood, D. and Hames, B. D., eds. (Oxford University Press: New York, 1992)), and vary according to the particular cell selected. Commercially available medium can be utilized. Non-limiting examples of medium include, for example, Dulbecco's Modified Eagle Medium (DMEM, Life Technologies), Minimal Essential Medium (MEM, Sigma, St. Louis, Mo.); HyClone cell culture medium (HyClone, Logan, Utah); and serum-free basal epithelial medium (CellnTech).

The media described above can be supplemented as necessary with supplementary components or ingredients, including optional components, in appropriate concentrations or amounts, as necessary or desired. Cell medium solutions provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range.

The medium also can be supplemented electively with one or more components from any of the following categories: (1) salts, for example, magnesium, calcium, and phosphate; (2) hormones and other growth factors such as, serum, insulin, transferrin, epidermal growth factor and fibroblast growth factor; (3) protein and tissue hydrolysates, for example peptone or peptone mixtures which can be obtained from purified gelatin, plant material, or animal byproducts; (4) nucleosides and bases such as, adenosine, thymidine, and hypoxanthine; (5) buffers, such as HEPES; (6) antibiotics, such as gentamycin or ampicillin; (7) cell protective agents, for example, pluronic polyol; and (8) galactose.

The mammalian cell culture that can be used with the present invention is prepared in a medium suitable for the particular cell being cultured. In one embodiment, the culture medium can be one of the aforementioned (for example, DMEM) that is supplemented with serum from a mammalian source (for example, fetal bovine serum (FBS)). For example, DMEM supplemented with FBS can be used to sustain the growth of embryonic fibroblasts, dermal fibroblasts or human foreskin fibroblasts. In another embodiment, the medium can be serum-free basal epithelial medium. For example, serum-free basal epithelial medium can used to sustain the growth of epithelial cells obtained from the reprogramming of fibroblast cells. In further embodiments, serum-free basal epithelial medium contains epidermal growth factor (EGF), fibroblast growth factor (FGF), or a combination thereof.

In one embodiment, fibroblasts cultured in an acceptable medium (such as DMEM supplemented with FBS), can be transduced with DNA vectors harboring genes that encode a protein of interest (such as Oct4, Sox2, Klf4 or c-Myc, or a combination thereof). In one embodiment, following transduction with DNA vectors harboring genes that encode a protein of interest (such as Oct4, Sox2, Klf4 or c-Myc, or a combination thereof), fibroblasts are incubated for at least 24 hours at about 37° C. In another embodiment, cells are incubated for at least 48, 72, or 96 hours, following transduction. Cells are incubated at about 35° C., about 36° C., about 37° C., about 38° C., or about 39° C.

In one embodiment, following transduction of fibroblasts with DNA vectors harboring genes that encode a protein of interest (such as Oct4, Sox2, Klf4 or c-Myc, or a combination thereof), the medium used to sustain the growth of fibroblasts is switched to serum-free basal epithelial medium. In a further embodiments, the serum-free basal epithelial medium contains EGF, FGF or a combination thereof. In another embodiment, following transduction with DNA vectors harboring genes that encode a protein of interest (such as Oct4, Sox2, Klf4 or c-Myc, or a combination thereof), fibroblasts are reprogrammed to epithelial cells. For example, the epithelial cells are induced epithelial cells.

Cells maintained in culture can be passaged by their transfer from a previous culture to a culture with fresh medium. In one embodiment, induced epithelial cells are stably maintained in cell culture for at least 3 passages, at least 4 passages, at least 5 passages, at least 6 passages, at least 7 passages, at least 8 passages, at least 9 passages, at least 10 passages, at least 11 passages, at least 12 passages, at least 13 passages, at least 14 passages, at least 15 passages, at least 20 passages, at least 25 passages, or at least 30 passages.

The cells suitable for culturing according to the methods of the present invention can harbor introduced expression vectors (constructs), such as plasmids and the like. The expression vector constructs can be introduced via transformation, microinjection, transfection, lipofection, electroporation, or infection. The expression vectors can contain coding sequences, or portions thereof, encoding the proteins for expression and production. Expression vectors containing sequences encoding the produced proteins and polypeptides, as well as the appropriate transcriptional and translational control elements, can be generated using methods well known to and practiced by those skilled in the art. These methods include synthetic techniques, in vitro recombinant DNA techniques, and in vivo genetic recombination which are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

In one embodiment, induced epithelial cells can express a variety of markers that distinguish them from fibroblasts. These markers include, but are not limited to cytokeratin 5 (CK5), CK8, CK14, CK18, beta-catenin, E-cadherin, Epithelial Membrane Antigen (EMA/Muc1), or EpCAM or a combination thereof. Expression of markers can be evaluated by a variety of methods known in the art. The presence of markers can be determined at the DNA, RNA or polypeptide level.

In one embodiment, the method can comprise detecting the presence of a marker gene (such as, CK5, CK8, CK14, CK18, beta-catenin or E-cadherin) polypeptide expression. Polypeptide expression includes the presence of a marker gene polypeptide sequence, or the presence of an elevated quantity of marker gene polypeptide as compared to non-epithelial cells. These can be detected by various techniques known in the art, including by sequencing and/or binding to specific ligands (such as antibodies). For example, polypeptide expression maybe evaluated by methods including, but not limited to, immunostaining, FACS analysis, or Western blot. These methods are well known in the art (for example, U.S. Pat. No. 8,004,661, U.S. Pat. No. 5,367,474, U.S. Pat. No. 4,347,935) and are described in T. S. Hawley & R. G. Hawley, 2005, Methods in Molecular Biology Volume 263: Flow Cytometry Protocols, Humana Press Inc; I. B. Buchwalow & W. BoEcker, 2010, Immunohistochemistry: Basics & Methods, Springer, Medford, Mass.; O. J. Bjerrum & N. H. H. Heegaard, 2009, Western Blotting: Immunoblotting, John Wiley & Sons, Chichester, UK.

In another embodiment, the method can comprise detecting the presence of marker gene (CK5, CK8, CK14, CK18, beta-catenin or E-cadherin) RNA expression, for example in reconstituted induced epithelial cells. RNA expression includes the presence of an RNA sequence, the presence of an RNA splicing or processing, or the presence of a quantity of RNA. These can be detected by various techniques known in the art, including by sequencing all or part of the marker gene RNA, or by selective hybridization or selective amplification of all or part of the RNA.

In one embodiment, following transduction of fibroblasts with DNA vectors harboring genes that encode a protein of interest (such as Oct4, Sox2, Klf4 or c-Myc, or a combination thereof), the medium used to sustain the growth of fibroblasts is switched to stem cell media. In a further embodiments, stem cell media is mouse embryonic stem cell media. In further embodiments, the stem cell media contains LIF, In another embodiment, following transduction with DNA vectors harboring genes that encode a protein of interest (such as Oct4, Sox2, Klf4 or c-Myc, or a combination thereof), fibroblasts are reprogrammed to induced pluripotent stem cells (iPSCs).

Cells maintained in culture can be passaged by their transfer from a previous culture to a culture with fresh medium. In one embodiment, iPSCs are stably maintained in cell culture for at least 3 passages, at least 4 passages, at least 5 passages, at least 6 passages, at least 7 passages, at least 8 passages, at least 9 passages, at least 10 passages, at least 11 passages, at least 12 passages, at least 13 passages, at least 14 passages, at least 15 passages, at least 20 passages, at least 25 passages, or at least 30 passages.

Methods for Reconstituting Induced Epithelial Cells into an Organ Tissue

A eukaryotic expression vector can be introduced into cells in order to produce proteins (for example, Nkx3.1, Androgen receptor (AR), FOXA1, FOXA2, KLF5, Pparγ, Grhl3, Ovo1, Foxa1, Elf3, Ehf) encoded by nucleotide sequences of the vector. Cells (such as induced epithelial cells) can harbor an expression vector (for example, one that contains a gene encoding Nkx3.1, AR, FOXA1, FOXA2, KLF5, Pparγ, Grhl3, Ovo1, Foxa1, Elf3, or Ehf) via introducing the expression vector into an appropriate host cell via methods known in the art.

An exogenous nucleic acid can be introduced into a cell via a variety of techniques known in the art. For example, a retrovirus can be used to introduce a nucleotide sequence into cells (such as induced epithelial cells). In one embodiment, the retrovirus is a Rebna retrovirus. In another embodiment, the retrovirus is a lentivirus. In yet another embodiment, the retrovirus is a LZRS retrovirus. Other viral vectors known in the art can be used to introduce a nucleotide sequence, including, but not limited to a lentivirus, a adenovirus, or a adeno-associated virus.

In one embodiment, a retrovirus can be used to introduce a nucleotide sequence into induced epithelial cells to produce proteins encoded by said nucleotide sequences (for example, Nkx3.1, AR, FOXA1, FOXA2, KLF5, Pparγ, Grhl3, Ovo1, Foxa1, Elf3, or Ehf). For example, the LZRS retrovirus, or a lentivirus, is used to introduce DNA into an induced epithelial cells to confer high-level stable expression of master regulatory genes (for example, Nkx3.1, AR, FOXA1, FOXA2, KLF5, Pparγ, Grhl3, Ovo1, Foxa1, Elf3, or Ehf). The nucleic acid of interest can encode only a single protein (for example, Nkx3.1, AR, FOXA1, FOXA2, KLF5, Pparγ, Grhl3, Ovo1, Foxa1, Elf3, or Ehf), or can encode for more than one protein of interest (for example, combinations of Nkx3.1, AR, FOXA1, FOXA2, KLF5, Pparγ, Grhl3, Ovo1, Foxa1, Elf3, or Ehf).

In one embodiment, induced epithelial cells can be transduced with DNA vectors harboring genes that encode a master regulatory gene. For example, a master regulatory gene can be a master regulatory gene for prostate development, such as Nkx3.1, AR, FOXA1, FOXA2, or a combination thereof. In another embodiment, a master regulatory gene can be a master regulatory gene for bladder development, such as KLF5, Pparγ, Grhl3, Ovo1, Foxa1, Elf3, Ehf, or a combination thereof. Master regulatory genes include, but are not limited to, XBP1, FOXA1, ACAD8, NKX3.1, MAP2K1, CREB3L4, HIPK2, YWHAQ, RIPK2, CREB3, FOXM1, TRIP13, CENPF, MEF2C, and ZNF423.

An exogenous nucleic acid can be introduced into a cell via a variety of techniques known in the art, such as lipofection, microinjection, calcium phosphate or calcium chloride precipitation, DEAE-dextrin-mediated transfection, or electroporation. Other methods used to transfect cells can also include calcium phosphate precipitation, modified calcium phosphate precipitation, polybrene precipitation, microinjection liposome fusion, and receptor-mediated gene delivery.

Cells to be genetically engineered can be primary and secondary cells, which can be obtained from various tissues and include cell types which can be maintained and propagated in culture. In one embodiment, cells are induced epithelial cells which can be obtained by the methods described by this invention.

In one embodiment, following transduction of induced epithelial cells with DNA vectors harboring genes that encode a master regulatory gene, cells are recombined with mesenchymal cells and a graft is performed in a subject. Tissue recombination assays are well known to one in the art (A14-A21). In one example, the mesenchymal cells comprise urogenital mesenchyme. In another example, the mesenchymal cells comprise embryonic bladder mesenchyme. Various routes of administration and various sites of graft can be utilized, such as, a renal graft, in order to introduced the transduced recombined cells into a site of preference. Once implanted into a subject (such as, a mouse, rat, or human), the transduced recombined cells can reconstitute into an organ tissue (such as, prostate epithelial tissue, or bladder epithelial tissue). In one example the graft is a renal graft. Administration of the recombined cells is not restricted to a single route, but may encompass administration by multiple routes. Exemplary administrations include a renal graft. Other modes of administration by multiple routes will be apparent to the skilled artisan.

In some embodiments, the cells used for administration will generally be subject-specific genetically engineered cells. In another embodiment, cells obtained from a different species or another individual of the same species can be used. Thus, using such cells may require administering an immunosuppressant to prevent rejection of the administered cells. Such methods have also been described in United States Patent Application Publication 2004/0057937 and PCT application publication WO 2001/32840, and are hereby incorporated by reference.

In one embodiment, cells may be introduced into an immunodeficient subject. For example, the cells may be introduced into an immunodeficient mouse such as an athymic nude mouse, a BALB/c nude mouse, a CD-1 nude mouse, a Fox Chase SCID beige mouse, a Fox Chase SCID mouse, a NIH-III nude mouse, a NOD SCID mouse, a NU/NU nude mouse, a SCID hairless congenic mouse, or a SCID hairless outbred mouse.

In one embodiment, induced epithelial cells are reconstituted into an organ tissue. For example, induced epithelial cells can be reconstituted into prostate epithelial tissue. In another example, induced epithelial cells can be reconstituted into bladder epithelial tissue. In one embodiment, reconstituted organ tissue can express a variety of markers that distinguish them as, for example, prostate epithelial tissue, or bladder epithelial tissue. These markers include, but are not limited to p63, CK5, AR, CK8, NKX3.1, PSA, Probasin, uroplakins or a combination thereof.

Expression of markers can be evaluated by a variety of methods known in the art. The presence of markers can be determined at the DNA, RNA or polypeptide level. In one embodiment, the method can comprise detecting the presence of a marker gene polypeptide expression. Polypeptide expression includes the presence of a marker gene polypeptide sequence, or the presence of an elevated quantity of marker gene polypeptide as compared to non-epithelial cells. These can be detected by various techniques known in the art, including by sequencing and/or binding to specific ligands (such as antibodies). For example, polypeptide expression maybe evaluated by methods including, but not limited to, immunostaining, FACS analysis, or Western blot. These methods are well known in the art (for example, U.S. Pat. No. 8,004,661, U.S. Pat. No. 5,367,474, U.S. Pat. No. 4,347,935) and are described in T. S. Hawley & R. G. Hawley, 2005, Methods in Molecular Biology Volume 263: Flow Cytometry Protocols, Humana Press Inc; I. B. Buchwalow & W. BoEcker, 2010, Immunohistochemistry: Basics & Methods, Springer, Medford, Mass.; O. J. Bjerrum & N. H. H. Heegaard, 2009, Western Blotting: Immunoblotting, John Wiley & Sons, Chichester, UK.

In another embodiment, the method can comprise detecting the presence of marker gene (such as, p63, CK5, AR, CK8, Probasin, or a combination thereof) RNA expression, for example in reconstituted organ tissue. RNA expression includes the presence of an RNA sequence, the presence of an RNA splicing or processing, or the presence of a quantity of RNA. These can be detected by various techniques known in the art, including by sequencing all or part of the marker gene RNA, or by selective hybridization or selective amplification of all or part of the RNA.

In another embodiment, reconstituted organ tissue can express markers that reveal reconstituted organ tissue architecture and are localized to specific areas. For example, the method can comprise detecting the presence of a marker gene (for example, p63, CK5, or a combination thereof) in the basal layer of prostate epithelial tissue, or bladder epithelial tissue. In another example, the method can comprise detecting the presence of a marker gene (for example, AR, CK8, or a combination thereof) in the luminal layer of prostate epithelial tissue. In a further example, the method can comprise detecting the presence of a marker gene (for example, CK8) in the luminal layer of bladder epithelial tissue. These can be detected by various techniques known in the art, including by sequencing and/or binding to specific ligands (such as antibodies). For example, marker gene expression can be evaluated by immunostaining. Other markers that known in the art that reveal reconstituted organ tissue architecture can also be used.

In one embodiment, reconstituted organ tissue can express markers that reveal reconstituted organ tissue functionality. For example, the method can comprise detecting the presence of a marker gene (for example, Probasin) in prostate epithelial tissue. These can be detected by various techniques known in the art, including by sequencing and/or binding to specific ligands (such as antibodies). For example, marker gene expression can be evaluated by immunostaining.

In one embodiment, reconstituted organ tissue can display characteristic tissue architecture. For example, reconstituted bladder epithelium can stain positive for the presence of the sub-epithelial connective tissue layer (lamina propria) surrounding the urothelium with Gomori's trichrome. The method can comprise detecting other characteristic tissue architecture in reconstituted organ tissue using various techniques known in the art, including staining of tissue with various stains including, but not limited to, Gomori's trichrome, haematoxylin and eosin, periodic acid-Schiff, Masson's trichrome, Silver staining, or Sudan staining.

Methods for Reconstituting Induced Pluripotent Stem Cells (iPSCs) into an Organ Tissue

In one embodiment, following the reprogramming of fibroblasts into iPSCs, iPSCs are recombined with mesenchymal cells and a graft is performed in a subject. Tissue recombination assays are well known to one in the art (A14-A21). In one example, the mesenchymal cells comprise urogenital mesenchyme. In another example, the mesenchymal cells comprise embryonic bladder mesenchyme. Various routes of administration and various sites of graft can be utilized, such as, a renal graft, in order to introduced the transduced recombined cells into a site of preference. Once implanted into a subject (such as, a mouse, rat, or human), the iPSCs can reconstitute into an organ tissue (such as, prostate epithelial tissue, or bladder epithelial tissue). In one example the graft is a renal graft. Administration of the recombined cells is not restricted to a single route, but may encompass administration by multiple routes. Exemplary administrations include a renal graft. Other modes of administration by multiple routes will be apparent to the skilled artisan.

In another embodiment, following the reprogramming of fibroblasts into iPSCs, the medium used to sustain the growth of iPSCs is switched to endodermal differentiation media. In one embodiment, the endodermal differentiation media contains Activin A, Noggin, and a GSK3β inhibitor. In one embodiment, iPSCs expressing endodermal markers are isolated. For example, endodermal markers include, but are not limited to GATA6. In one embodiment, the iPSCs express GATA6. The methods for separating, enriching, isolating or purifying iPSCs expressing endodermal markers according to the invention may be combined with other methods for separating, enriching, isolating or purifying cells that are known in the art. The presence of markers can be determined at the DNA, RNA or polypeptide level. In one embodiment, following the isolation of iPSCs expressing endodermal markers (e.g. GATA6), the iPSCs are recombined with mesenchymal cells and a graft is performed in a subject. In one embodiment, the iPSCs are cultured in a three-dimensional culture. In one embodiment, the iPSCs are cultured in Matrigel.

In some embodiments, the cells used for administration will generally be subject-specific genetically engineered cells. In another embodiment, cells obtained from a different species or another individual of the same species can be used. Thus, using such cells may require administering an immunosuppressant to prevent rejection of the administered cells. Such methods have also been described in United States Patent Application Publication 2004/0057937 and PCT application publication WO 2001/32840, and are hereby incorporated by reference.

In one embodiment, cells may be introduced into an immunodeficient subject. For example, the cells may be introduced into an immunodeficient mouse such as an athymic nude mouse, a BALB/c nude mouse, a CD-1 nude mouse, a Fox Chase SCID beige mouse, a Fox Chase SCID mouse, a NIH-III nude mouse, a NOD SCID mouse, a NU/NU nude mouse, a SCID hairless congenic mouse, or a SCID hairless outbred mouse.

In one embodiment, iPSCs are reconstituted into an organ tissue. For example, iPSCs can be reconstituted into prostate epithelial tissue. In another example, iPSCs can be reconstituted into bladder epithelial tissue. In one embodiment, reconstituted organ tissue can express a variety of markers that distinguish them as, for example, prostate epithelial tissue, or bladder epithelial tissue. These markers include, but are not limited to p63, CK5, AR, CK8, NKX3.1, PSA, Probasin, uroplakins or a combination thereof

In one embodiment, iPSCs expressing an endodermal marker are reconstituted into an organ tissue. For example, iPSCs expressing an endodermal marker can be reconstituted into prostate epithelial tissue. In another example, iPSCs expressing an endodermal marker can be reconstituted into bladder epithelial tissue. In one embodiment, reconstituted organ tissue can express a variety of markers that distinguish them as, for example, prostate epithelial tissue, or bladder epithelial tissue. These markers include, but are not limited to p63, CK5, AR, CK8, NKX3.1, PSA, Probasin, uroplakins or a combination thereof.

Expression of markers can be evaluated by a variety of methods known in the art. The presence of markers can be determined at the DNA, RNA or polypeptide level. In one embodiment, the method can comprise detecting the presence of a marker gene polypeptide expression. Polypeptide expression includes the presence of a marker gene polypeptide sequence, or the presence of an elevated quantity of marker gene polypeptide as compared to non-epithelial cells. These can be detected by various techniques known in the art, including by sequencing and/or binding to specific ligands (such as antibodies). For example, polypeptide expression maybe evaluated by methods including, but not limited to, immunostaining, FACS analysis, or Western blot. These methods are well known in the art (for example, U.S. Pat. No. 8,004,661, U.S. Pat. No. 5,367,474, U.S. Pat. No. 4,347,935) and are described in T. S. Hawley & R. G. Hawley, 2005, Methods in Molecular Biology Volume 263: Flow Cytometry Protocols, Humana Press Inc; I. B. Buchwalow & W. BoEcker, 2010, Immunohistochemistry: Basics & Methods, Springer, Medford, Mass.; O. J. Bjerrum & N. H. H. Heegaard, 2009, Western Blotting: Immunoblotting, John Wiley & Sons, Chichester, UK.

In another embodiment, the method can comprise detecting the presence of marker gene (such as, p63, CK5, AR, CK8, Probasin, or a combination thereof) RNA expression, for example in reconstituted organ tissue. RNA expression includes the presence of an RNA sequence, the presence of an RNA splicing or processing, or the presence of a quantity of RNA. These can be detected by various techniques known in the art, including by sequencing all or part of the marker gene RNA, or by selective hybridization or selective amplification of all or part of the RNA.

In another embodiment, reconstituted organ tissue can express markers that reveal reconstituted organ tissue architecture and are localized to specific areas. For example, the method can comprise detecting the presence of a marker gene (for example, p63, CK5, or a combination thereof) in the basal layer of prostate epithelial tissue, or bladder epithelial tissue. In another example, the method can comprise detecting the presence of a marker gene (for example, AR, CK8, or a combination thereof) in the luminal layer of prostate epithelial tissue. In a further example, the method can comprise detecting the presence of a marker gene (for example, CK8) in the luminal layer of bladder epithelial tissue. These can be detected by various techniques known in the art, including by sequencing and/or binding to specific ligands (such as antibodies). For example, marker gene expression can be evaluated by immunostaining. Other markers that known in the art that reveal reconstituted organ tissue architecture can also be used.

In one embodiment, reconstituted organ tissue can express markers that reveal reconstituted organ tissue functionality. For example, the method can comprise detecting the presence of a marker gene (for example, Probasin) in prostate epithelial tissue. These can be detected by various techniques known in the art, including by sequencing and/or binding to specific ligands (such as antibodies). For example, marker gene expression can be evaluated by immunostaining.

In one embodiment, reconstituted organ tissue can display characteristic tissue architecture. For example, reconstituted bladder epithelium can stain positive for the presence of the sub-epithelial connective tissue layer (lamina propria) surrounding the urothelium with Gomori's trichrome. The method can comprise detecting other characteristic tissue architecture in reconstituted organ tissue using various techniques known in the art, including staining of tissue with various stains including, but not limited to, Gomori's trichrome, haematoxylin and eosin, periodic acid-Schiff, Masson's trichrome, Silver staining, or Sudan staining.

An aspect of the invention is directed to a method for transdifferentiation of embryonic fibroblast cells into an organ tissue, the method comprising: (a) isolating embryonic fibroblasts (EFs); (b) transducing EFs with a retrovirus comprising a reprogramming factor; (c) culturing the infected EFs in stem cell media for at least 24 hours at about 37° C. to generate induced pluripotent stem cells (iPSCs); (d) isolating iPSCs; (e) recombining the cells of (d) with mesenchymal cells; and (f) performing a graft of the recombined cells of (e) into an immunodeficient subject. In one embodiment, the stem cell media comprises LIF. In one embodiment, the graft is maintained in the subject for about 6 to 8 weeks. In one embodiment, the mesenchymal cells comprise urogenital mesenchyme. In one embodiment, the mesenchymal cells comprise bladder mesenchyme. In one embodiment, the graft is a renal graft. In one embodiment, the organ tissue is prostate epithelial tissue. In one embodiment, the organ tissue is bladder epithelial tissue. In one embodiment, the prostate tissue expresses p63, CK5, or a combination thereof, in the basal layer. In one embodiment, the bladder tissue expresses p63, CK5, or a combination thereof, in the basal layer. In one embodiment, the prostate tissue expresses AR, CK8, or a combination thereof, in the luminal layer. In one embodiment, the prostate tissue expresses Probasin, PSA, or a combination thereof. In one embodiment, the bladder tissue expresses CK8, uroplakins, or a combination thereof. In one embodiment, the bladder tissue stains positive for the presence of the sub-epithelial connective tissue layer (lamina propria) surrounding the urothelium with Gomori's trichrome. In one embodiment, the retrovirus is a lentivirus. In one embodiment, the lentivirus is doxycycline regulated.

An aspect of the invention is directed to a method for differentiation of induced pluripotent stem cells (iPSCs) into an organ tissue, the method comprising: (a) isolating iPSCs; (b) recombining the cells of (a) with mesenchymal cells; and (c) performing a graft of the recombined cells of (b) into an immunodeficient subject. In one embodiment, the graft is maintained in the subject for about 6 to 8 weeks. In one embodiment, the mesenchymal cells comprise urogenital mesenchyme. In one embodiment, the mesenchymal cells comprise bladder mesenchyme. In one embodiment, the graft is a renal graft. In one embodiment, the organ tissue is prostate epithelial tissue. In one embodiment, the organ tissue is bladder epithelial tissue. In one embodiment, the prostate tissue expresses p63, CK5, or a combination thereof, in the basal layer. In one embodiment, the bladder tissue expresses p63, CK5, or a combination thereof, in the basal layer. In one embodiment, the prostate tissue expresses AR, CK8, or a combination thereof, in the luminal layer. In one embodiment, the prostate tissue expresses Probasin, PSA, or a combination thereof. In one embodiment, the bladder tissue expresses CK8, uroplakins, or a combination thereof. In one embodiment, the bladder tissue stains positive for the presence of the sub-epithelial connective tissue layer (lamina propria) surrounding the urothelium with Gomori's trichrome.

An aspect of the invention is directed to a method for differentiation of induced pluripotent stem cells (iPSCs) into an organ tissue, the method comprising: (a) isolating iPSCs; (b) culturing iPSCs in endodermal differentiation media; (c) isolating iPSCs that express an endodermal marker; (d) recombining the cells of (c) with mesenchymal cells; and (e) performing a graft of the recombined cells of (d) into an immunodeficient subject. In one embodiment, the endodermal differentiation media contains Activin A, Noggin, and a GSK3β inhibitor. In another embodiment, the endodermal marker is GATA6. In one embodiment, the iPSCs are cultured in a three-dimensional culture. In one embodiment, the iPSCs are cultured in Matrigel. In another embodiment, the graft is maintained in the subject for about 6 to 8 weeks. In another embodiment, the mesenchymal cells comprise urogenital mesenchyme. In another embodiment, the mesenchymal cells comprise bladder mesenchyme. In another embodiment, the graft is a renal graft. In another embodiment, the organ tissue is prostate epithelial tissue. In another embodiment, the organ tissue is bladder epithelial tissue. In another embodiment, the prostate tissue expresses p63, CK5, or a combination thereof, in the basal layer. In another embodiment, the bladder tissue expresses p63, CK5, or a combination thereof, in the basal layer. In another embodiment, the prostate tissue expresses AR, CK8, or a combination thereof, in the luminal layer. In another embodiment, the prostate tissue expresses Probasin, PSA, or a combination thereof. In another embodiment, the bladder tissue expresses CK8, uroplakins, or a combination thereof. In another embodiment, the bladder tissue stains positive for the presence of the sub-epithelial connective tissue layer (lamina propria) surrounding the urothelium with Gomori's trichrome.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Human and Mouse Prostate Interactomes

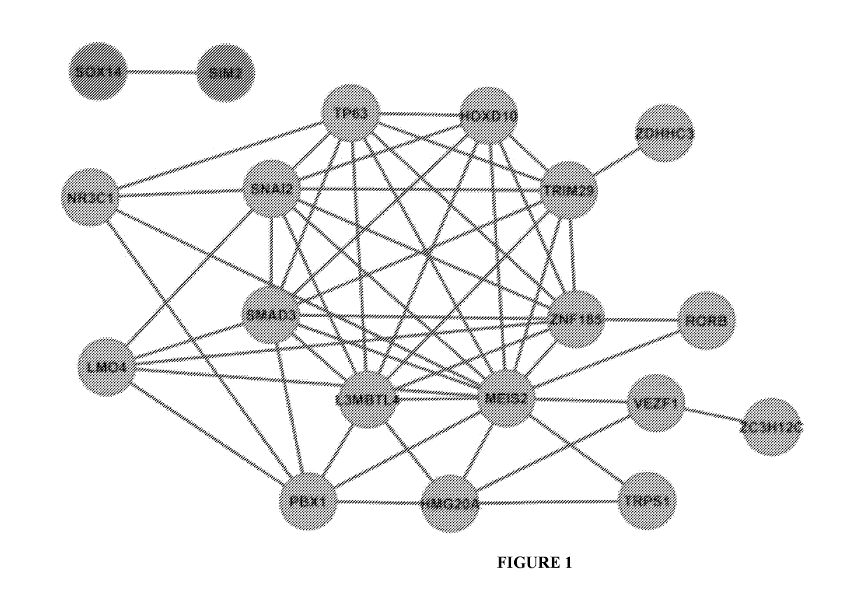

Interactomes have been generated for mouse and human prostate tissue, using an established algorithm for reverse engineering, such as ARACNe [15-17]. The mouse prostate interactome was constructed using a large collection of gene expression profiles from drug-induced perturbation of several transgenic models, with phenotypes ranging from normal tissue to advanced prostate cancer. The human prostate cancer interactome was constructed from a large published dataset comprised of prostate cancer specimens and adjacent normal tissue [37]. These interactomes, which are being validated using cell culture assays, have been interrogated to identify master regulator genes for prostate cancer initiation, using the MARINa algorithm [18, 19] (FIG. 1).

### Example 2

### Generation of Stable “Primitive” Epithelial Cells from Fibroblasts In Vitro without an Intervening Pluripotent State

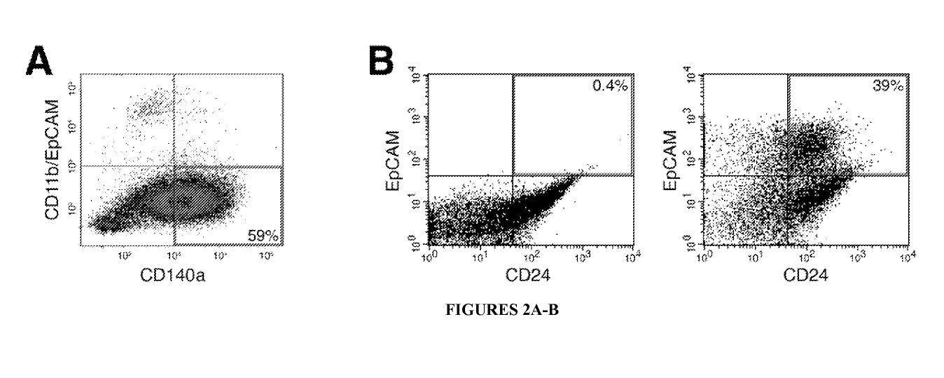

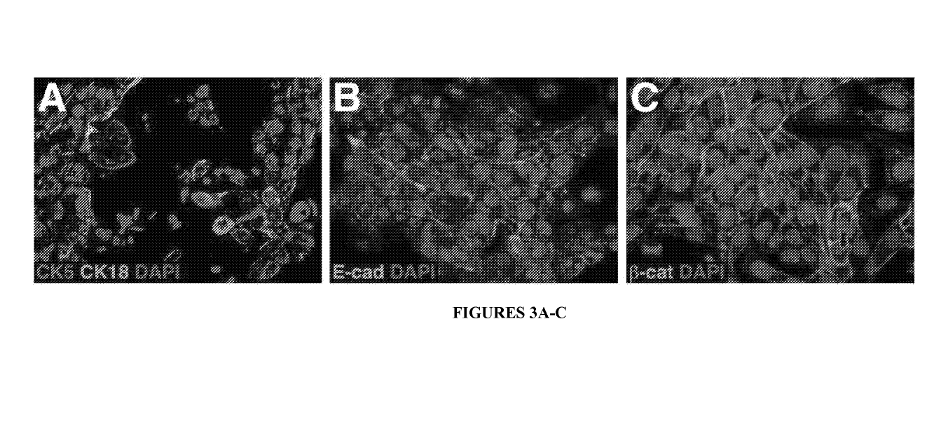

Expression of reprogramming factors have been used in fibroblasts to generate cells with epithelial morphologies in culture. Mouse embryonic fibroblasts (MEFs) of distinct genotypes (wild-type, Oct4-GFP knock-in, and Nkx3.1-lacZ knock-in) have been derived from E13.5 mouse embryos after the head and pelvis were removed to exclude neural and prostate progenitors. These MEFs were used after sorting for the mesenchymal marker CD140 or sorting against Lin/Mac-1(CD11b)/EpCAM markers to exclude blood, endothelial, and epithelial contaminants, thereby reducing the heterogeneity of the primary fibroblast population (FIG. 2A). Following infection of MEFs with Rebna retroviruses conferring high-level stable expression of reprogramming factors (Oct4, Sox2, Klf4, and c-Myc=OSKM); morphological changes were observed at 48 hours post-infection, at which time the culture medium was switched to serum-free basal epithelial medium containing EGF and FGF. Under these conditions, approximately 40% of cells were EpCAM⁺CD24⁺ (FIG. 2B), displayed epithelial morphology and positive immunoreactivity for cytokeratin 5 (CK5), CK8, CK14, CK18, beta-catenin, and E-cadherin, and could be stably maintained for multiple passages (FIG. 3). Thus, these reprogrammed epithelial cells display phenotypes that are likely to be distinct from those of the transient cells generated by a mesenchymal-to-epithelial transition (MET) at early phases of induced pluripotent stem cell (iPSC) formation [38, 39]. In addition, to exclude the possibility that the mouse embryonic fibroblasts (MEFs) had been reprogrammed to a pluripotent state followed by differentiation to epithelial fates, a control experiment was performed using Oct4-GFP knock-in MEFs. Following retroviral infection of these MEFs, GFP⁺ cells were not observed in epithelial basal medium, while the same cultures placed in mESC/LIF medium showed rapid formation of GFP⁺ colonies with the morphological features of iPSC, indicating that the reprogrammed epithelial cells did not transit through a pluripotent state.

### Example 3

### Directed Differentiation of “Primitive” Epithelial Cells to Prostate Epithelium

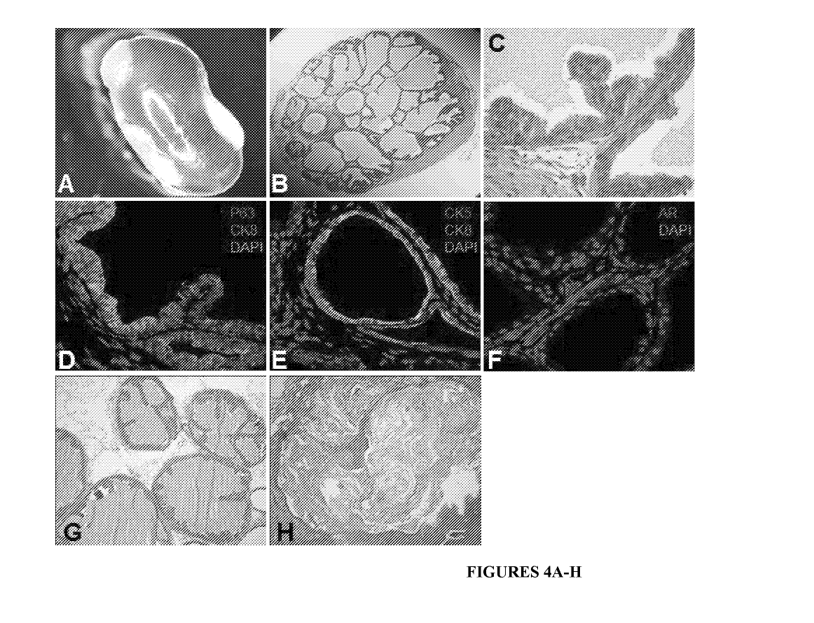

The “primitive” epithelial cells were further stably transduced with Nkx3.1 and AR-known master regulators of prostate development followed by tissue recombination assays with rat UGM in renal grafts (FIG. 4A). The combination of prostate specific master regulators and prostate inductive mesenchyme was able to determine complete differentiation of the iEpi into prostatic tissue (FIGS. 4B-C). Immunostaining revealed proper tissue architecture with a basal layer positive for p63 and CK5 and a luminal layer positive for CK8/CK18 and AR (FIGS. 4D-F). Freshly isolated mouse prostate epithelial cells were used as controls (FIG. 4G). In contrast, in the absence of the prostate specific genes, OSKM-induced primitive epithelial cells assumed a more general epithelial fate and produced teratomas which were 90% composed of epithelial cells generating large amounts of keratin (FIG. 4H). This experiment validates the approach to generate prostate and bladder epithelium through direct conversion of fibroblasts without an intervening pluripotent state.

### Example 4

### Differentiation of Mouse iPSC into Prostate and Bladder Epithelium

Without being bound by theory, these studies can identify master regulator genes for the normal prostate epithelium by regulatory network analysis using existing or newly generated interactomes for mouse and human prostate and bladder tissue. Together with master regulators identified by the candidate gene approach, these genes can be used in gain- or loss-of-function experiments to promote prostate differentiation by mouse iPSC using an in vivo tissue recombination/renal grafting system.

Experimental Design:

To identify master regulators of prostate and bladder epithelium, expression signatures can first be generated for adult and embryonic mouse prostate epithelium and bladder urothelium as well as mammary epithelium as control comparisons. These signatures can be produced by gene expression profiling of six biological replicate samples using standard protocols and hybridization to Illumina BeadArrays. Alternatively, transcriptomes can be generated in a more comprehensive way through RNA-seq. These expression signatures can be used to interrogate the mouse prostate and bladder interactomes using the MARINa and MINDy algorithms to identify master regulator (MR) genes and their modulators, as previously reported [18, 19]. The algorithms infer direct and indirect interactions among specific gene products, mRNA and DNA sequences from statistically significant co-regulation data. The power of this approach lies in its basis on genome-wide gene expression profiles data gathered from biological samples and consideration for all genes equally. Thus it is unbiased, unlike other approaches relying on a priori knowledge and probabilistic assumptions about how genes interact. Without being bound by theory, additional putative master regulators can be inferred by a candidate gene approach (e.g., Nkx3.1, FoxA1, androgen receptor, KLF5, Pparγ and Grhl3), based upon biological and biochemical identification of key transcription factors for prostate and bladder development (e.g., [40]).

In the next step, validation of the identified candidate MRs can be performed. The ability of each candidate to affect the propensity for epithelial differentiation of induced pluripotent stem cell (iPSCs) can be tested. To determine whether these master regulators can enhance the differentiation of mouse iPSC, lentiviral infection can be used to overexpress positive master regulators or knock-down negative regulators, as appropriate. Synergistic master regulators can be identified using the approach described in [18, 19], and experimentally tested. To assess the ability of these iPSCs to differentiate into mature prostate epithelium in vivo, a tissue recombination system can be employed in which these cells can be combined with dissociated rat embryonic urogenital mesenchyme, followed by renal grafting into immunodeficient nude mice. This basic strategy was successfully used previously to explore prostate differentiation and stem cell function ([4, 41-43]). As positive controls, mouse ESC can be used as well as human ESC, since human ESC have been shown to generate prostate epithelial cells under similar conditions [5]. For induction of bladder urothelium, embryonic bladder mesenchyme can be used in a similar experimental setting. Immunostaining for specific tissue markers can be performed to confirm the prostatic (mouse Nkx3.1, mouse AR, prostate secretions) or urothelial (uroplakins) phenotype. Epithelial tissue architecture can be confirmed with immunostaining for basal (p63, CK5) and luminal (CK8) markers. Gomori's trichrome staining can be used to demonstrate the presence of the sub-epithelial connective tissue layer (lamina propria) surrounding the urothelium. SMA immunolocalization can be performed to visualize the outer smooth muscle layer. Prostate epithelium and bladder urothelium can be used as controls for both tissue recombination experiments and immunostainings. In addition, the transcriptional profile of the induced tissues can be compared with normal mouse tissues through DNA microarray analysis.

Without being bound by theory, the interactome analysis can highlight known regulators of tissue development, such as AR or KLF5 pathways, as well as new, context-specific gene regulatory networks. For example, new master regulatory genes involved in early stages of tissue commitment and differentiation can be uncovered and validated. Prostate and bladder epithelia can be generated in vivo in renal grafts. Uncontrolled cell proliferation determined by the positive master regulators in different cell compartments resulting in an unbalanced basal:luminal cell ratio and improper epithelial-mesenchymal interactions can result. For instance, overexpression of KLF5 in stratified epithelium determines proliferation of the basal compartment [3]. If this event would occur in the urothelium, a lentiviral tet-on/tet-off system can be used to transduce the tissue master regulators and downregulate them in vivo in renal grafts.

### Example 5

### Direct Conversion of Mouse Fibroblasts into Prostate and Bladder Epithelium

These studies can employ expression of pluripotency factors to promote the reprogramming of mouse embryonic fibroblasts (MEFs) to normal prostate epithelial cells without undergoing an intermediate pluripotent state followed by expression of tissue specific master regulators. One approach relies on retroviral expression of Oct4, Sox2, Klf4, and c-Myc in MEFs, while a second approach uses transient doxycycline-inducible expression of pluripotency factors in MEFs. In both cases, reprogrammed cells with epithelial characteristics can be isolated by flow cytometry and used for tissue recombination and renal grafting to assess prostate and bladder differentiation. In addition, these studies can seek to optimize reprogramming conditions in the absence of c-Myc to reduce oncogenic transformation of the resulting epithelial cells.

Experimental Design:

In initial studies, a system can be used in which the expression of reprogramming factors is regulated by administration of doxycycline, which allows temporal control over their expression and avoid issues associated with their continuous expression. In one approach, mouse embryonic fibroblasts (MEFs) can be derived, as well as dermal fibroblasts and keratinocytes, from mice carrying a doxycycline-regulated single-copy transgene expressing Oct4, Sox2, Klf4, and c-Myc as a polycistronic transcript [44]. In a second approach, doxycycline-regulated lentiviruses can be used for each of the reprogramming factors, which can allow their use of desired combinations of interest (for example, Oct4, Sox2, and Klf4, without c-Myc). Without being bound by theory, additional 1-factor and 2-factor combinations can allow systematic investigation of the mechanisms by which the epithelial switch is activated.

Following these initial studies, the functional properties of the reprogrammed epithelial cells can be examined. In particular, it can be determined whether they display characteristic features of epithelial growth using in vitro assays, such as growth in three-dimensional culture in Matrigel, in the presence or absence of stromal cells. Their growth can also be examined in anchorage-independent conditions promoting the growth of spheres or organoids, as have been previously described for prostate epithelial cells [45, 46]. Finally, gene expression profiling of these reprogrammed epithelial cells can be performed to determine their similarity to immature epithelial cell types (e.g. primitive urogenital epithelium). The gene signatures of the reprogrammed epithelial cells can also be compared under a variety of culture conditions and ascertain their similarity to signatures of mature epithelium from mouse prostate, bladder, and breast, using Principal Components Analysis (PCA) and Gene Set Enrichment Analysis (GSEA) [36, 47], which have previously been used in other studies [48].

To determine whether the master regulators can enhance the differentiation of reprogrammed epithelial cells in culture, lentiviral infection can be used to overexpress positive master regulators or knock-down negative regulators. The resulting reprogrammed cells can be assayed for their morphological features and marker expression, and cells with promising phenotypes can be analyzed by expression profiling for comparison to the gene signatures of normal prostate and bladder epithelium. To assess prostate and bladder differentiation, flow cytometry can be used to isolate EpCAM⁺/CD24⁺ reprogrammed epithelial cells that have been maintained in prostate basal medium, followed by lentiviral infection with master regulators, tissue recombination, and renal grafting. Renal grafts can be harvested at various time points post-implantation and the epithelial cells can be dissociated and FACS sorted. Expression profiles of epithelial cells can be generated in order to identify new factors involved in terminal differentiation of prostate and bladder tissue.

Without being bound by theory, reprogrammed epithelial cells can display properties of a “primitive” epithelial cell. Although it may be found that specific culture conditions do not promote their terminal differentiation or formation of organoid structures, tissue recombination assays provide an in vivo microenvironment that is more conducive to cellular differentiation.

### Example 6

### Generation of Induced Epithelial Cells from Reprogrammed Fibroblasts, and Terminal Differentiation in Prostate Tissue in Renal Grafts

Expression of reprogramming factors have been used in fibroblasts to generate cells with epithelial morphologies in culture. For this purpose, mouse embryonic fibroblasts (MEFs) of distinct genotypes (wild-type, Oct4-GFP knock-in, and Nkx3.1-lacZ knock-in) were derived from E13.5 mouse embryos after the head and pelvis were removed to exclude neural and prostate progenitors. These MEFs were used after sorting for the mesenchymal marker CD140 or sorting against Lin/Mac-1(CD11b)/EpCAM markers to exclude blood, endothelial, and epithelial contaminants, thereby reducing the heterogeneity of the primary fibroblast population (FIG. 1A). The MEFs were then infected with retroviruses conferring high-level stable expression of reprogramming factors (Oct4, Sox2, Klf4, and c-Myc=OSKM; these are contained in Rebna retroviruses). Morphological changes were observed at 48 hours post-infection, at which time the culture medium was switched to serum-free basal epithelial medium containing EGF and FGF (commercially available from CellnTech, cat. No CnT-12). Under these conditions, approximately 40% of cells were EpCAM⁺CD24⁺ (FIG. 1B), displayed epithelial morphology and positive immunoreactivity for cytokeratin 5 (CK5), CK8, CK14, CK18, beta-catenin, and E-cadherin, and could be stably maintained for multiple passages (FIG. 2).

These induced epithelial cells were further stably transduced with viruses expressing Nkx3.1 and AR or NKX3.1, AR and FOXA1, which are known master regulatory genes for prostate development, followed by tissue recombination assays with rat urogenital mesenchyme (UGM) in renal grafts in immunodeficient male mice (FIG. 3A). The combination of prostate specific master regulators and prostate inductive mesenchyme was able to specify complete differentiation of the induced epithelial cells into prostate tissue (FIG. 3B-C). Immunostaining revealed proper prostate tissue architecture with a basal layer positive for p63 and CK5 and a luminal layer positive for CK8, CK18, and AR (FIG. 3D-F). The tissue was also positive for Probasin (a prostate-specific secreted protein) indicating that the tissue was functional (FIG. 3G).

### Example 7

### Investigation of Direct Conversion of Mouse and Human Fibroblasts into Prostate Epithelium

A goal of stem cell biology is the creation of desired cell types and tissues, which can be achieved by directed differentiation from pluripotent cells, or alternatively by direct lineage conversion in which transdifferentiation of cell types occurs. While these approaches are utilized for applications in regenerative medicine, they can also be used as the basis for genetically-engineered models of human disease, including cancer. Without being bound by theory, direct lineage conversion can be used in combination with gene targeting methods for the creation of genetically-engineered human models of cancer. In this application, direct conversion and tissue recombination can be used to generate mouse and human prostate tissue, and this reprogramming methodology can be applied to generate human tumor tissue for modeling of prostate cancer. Mouse and human fibroblasts can be directly converted to prostate tissue using a three-step process involving transient induction of pluripotency factors, expression of master regulators of prostate epithelium, and tissue recombination with urogenital mesenchyme followed by renal grafting. This direct conversion approach can be used to analyze the molecular mechanisms of reprogramming to prostate tissue as well as to generate genetically-engineered human models of prostate cancer.

Without being bound by theory, the mechanisms of direct conversion and the generation of human models of prostate cancer can be investigated. For example, the direct conversion of mouse and human fibroblasts into prostate epithelium can be investigated by systems analyses to identify optimal master regulators of prostate epithelial differentiation and by molecular analyses of reprogrammed prostate tissue. Mechanisms of direct conversion to prostate epithelium can be analyzed by investigating the multiple steps of cellular reprogramming. These studies can determine whether there is a transient intermediate pluripotent state, identify the cell(s) of origin for reprogrammed prostate epithelium, and analyze the reprogramming activity of urogenital mesenchyme. Modeling of human prostate cancer initiation by gene targeting and direct conversion can be investigated using Transcription Activator-Like Effector nucleases (TALENs) for the specific alteration of tumor suppressor genes that are mutated in human prostate cancer, followed by generation of reprogrammed human prostate tissue. In combination, these studies can provide the basis for an innovative approach for human cancer modeling, which can yield insights into the molecular mechanisms of human prostate cancer initiation.

Without being bound by theory, the proposed studies can yield insights into the basis for direct lineage conversion and cellular reprogramming, which have multiple applications in regenerative medicine and disease modeling. For example, this can also provide the basis for an approach for generating genetically-engineered human models of prostate cancer, which can have important implications for understanding the molecular mechanisms of prostate cancer initiation and progression.

Mouse as well as human fibroblasts can be directly converted into epithelial cells in culture following transient expression of the four “pluripotency factors” (Oct4, Sox2, Klf4, c-Myc). Following expression of prostate regulatory genes such as androgen receptor (AR), FoxA1, and Nkx3.1 in these induced epithelial cells, and recombination with embryonic urogenital mesenchyme, the resulting renal grafts can generate histologically normal prostate tissue with appropriate expression of tissue-specific markers. TALENs have also been used for gene targeting in prostate epithelial cell lines. Computational/systems biology approaches have been used to construct genome-wide regulatory networks (interactomes) for mouse and human prostate tissue, which can allow identification of master regulator (MR) genes that govern prostate epithelial cell fates, and thereby promote optimization of the reprogramming process.

Based on these findings, and without being bound by theory, this direct conversion/transdifferentiation approach can be used successfully to generate normal human prostate tissue, and in combination with gene targeting approaches, can be used to generate genetically-engineered human models of prostate cancer. This experimental methodology can be validated and the mechanistic basis for the direct conversion process can be investigated. For example, the direct conversion of mouse and human fibroblasts into prostate epithelium can be investigated by the identification of master regulators (MRs) of prostate epithelial differentiation, and molecular analyses of the reprogrammed prostate tissue. These studies can employ systems analyses of mouse and human prostate gene regulatory networks to identify candidate MRs, followed by functional assessment of their ability to promote direct conversion. These studies can provide a comprehensive analysis of MR combinations for optimization of reprogramming to prostate epithelium.

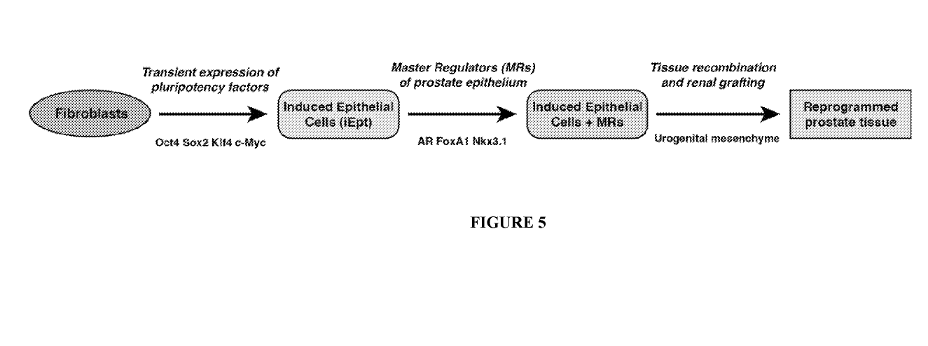

A general strategy for reprogramming to generate mouse and human prostate tissue has been developed (FIG. 5). As detailed herein, this strategy involves a three-step procedure in which: 1) transient expression of pluripotency factors is used to generate induced epithelial cells; 2) retroviral infection is used to express candidate master regulators of prostate epithelium; and 3) tissue recombination with embryonic urogenital mesenchyme followed by renal grafting is used to generate prostate tissue. Systems analyses of master regulators of prostate epithelium has been initiated, gene targeting in human cells using TALENs has been established.

Generation of Induced Epithelial Cells by Transient Expression of Pluripotency Factors:

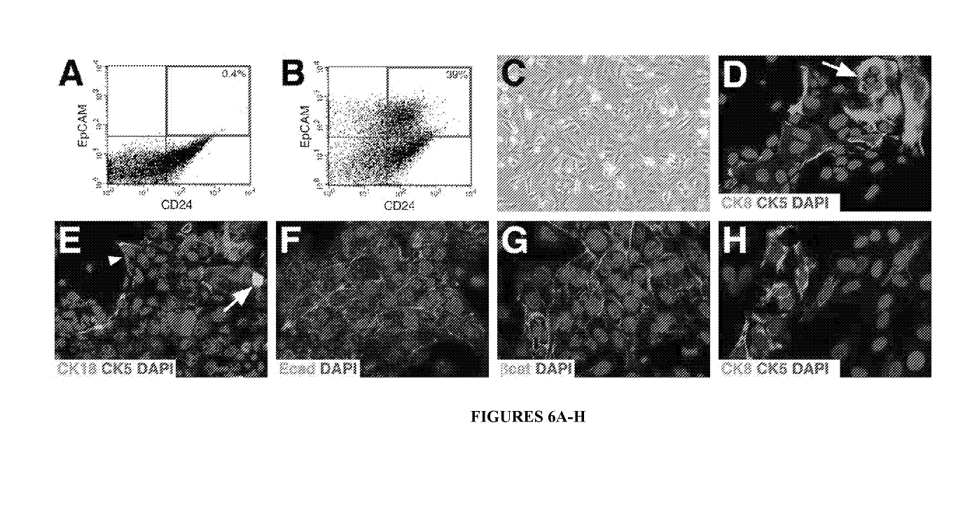

Expression of pluripotency factors in fibroblasts can induce the formation of cells with epithelial morphologies in culture, termed induced epithelial cells (iEpt) cells. Mouse embryonic fibroblasts (MEFs), generated from E13.5 limb buds of wild-type mice to exclude neural and prostate progenitors, as well as dermal fibroblasts (MDFs) from P0 mice, were used. These MEFs and MDFs were then flow-sorted for the mesenchymal marker CD140a and against Lin/Mac-1(CD11b)/EpCAM markers to exclude blood, endothelial, and epithelial contaminants, thereby reducing the heterogeneity of the fibroblast population (FIG. 6A). These sorted MEFs were infected with REBNA retroviruses [A41] conferring high-level constitutive expression of the Yamanaka reprogramming factors (OSKM: Oct4, Sox2, Klf4, and c-Myc). Morphological changes were observed in the infected fibroblasts at 48 hours post-infection, at which time the culture medium was switched to chemically-defined basal epithelial medium containing EGF and FGF (CellnTec). Under these conditions, approximately 40% of cells were EpCAM⁺CD24⁺ (FIG. 6B,C), displayed epithelial morphology and positive immunoreactivity for cytokeratin 5 (CK5), CK8, CK18, E-cadherin, and β-catenin, and could be stably maintained for several passages (FIG. 6D-G). Thus, these reprogrammed iEpt cells are distinct from the transient cells generated by a mesenchymal-to-epithelial transition (MET) at early phases of iPSC formation [A42, A43].

The system for the expression of reprogramming factors was changed to one that is regulated by administration of doxycycline, which allows temporal control over their expression and avoids issues associated with their continuous expression. In this approach, MEFs and MDFs were derived using the same strategy as above from mice carrying a doxycycline-regulated single-copy transgene expressing Oct4, Sox2, Klf4, and c-Myc as a polycistronic transcript [A44]. These fibroblast cultures were treated with doxycycline for 5-9 days to induce pluripotency factor expression, followed by 10 days in the absence of doxycycline to select for OSKM-independent iEpt cells. Under these conditions, approximately 10% of cells were EpCAM⁺CD24⁺ and displayed a stable epithelial morphology. The transient expression of OSKM can induce iEpt cells to form in basal epithelial medium.

Production of Mouse Prostate Tissue from Reprogrammed Fibroblasts by Tissue Recombination:

iEpt cells were investigated for their ability to be further reprogrammed to generate prostate tissue. The expression of putative master regulators (MRs) of prostate differentiation was combined with a tissue recombination assay. A candidate gene approach was used to select putative prostate epithelial MRs based upon biological and biochemical identification of key transcription factors for prostate development (e.g., [A45]). Androgen receptor (AR) was selected due to its central roles in prostate specification, organogenesis, and adult homeostasis and regeneration [A40, A46]. FoxA1 was selected because it is known to be critical for prostate development and functions as a pioneer factor in opening chromatin for AR binding [A45, A47-A50]. Nkx3.1 was selected due to its role in prostate development and luminal epithelial differentiation, and its participation in many AR transcriptional complexes [A16, A45, A51, A52].

Using retroviruses that constitutively express AR, FoxA1, and Nkx3.1 [A19, A53], the ability of iEpt cells to form prostate tissue following recombination with urogenital mesenchyme was investigated. Urogenital mesenchyme from E18.5 rat embryos and renal grafting in immunodeficient NCR nude mice (Taconic), using between 50,000 and 250,000 iEpt cells together with 250,000 mesenchymal cells, was used. To determine the contribution of each MR to prostate tissue formation, iEpt cells that received different combinations and proportions of these factors were used. iEpt cells were generated using the constitutively-expressed OSKM factors with retroviruses expressing AR, FoxA1, or Nkx3.1 individually, or in combination. The resulting renal grafts were harvested after 6-8 weeks, and analyzed by hematoxylin-eosin staining and immunostaining for specific markers. As positive controls, adult mouse prostate epithelial cells in tissue recombinations performed in parallel were used. As negative controls, renal grafts were generated from iEpt cells in the absence of urogenital mesenchyme, which never formed prostate tissue, with or without prostate MR expression (n=0/11); instead, 9 of these grafts only formed teratomas, while the remaining 2 grafts formed teratomas with areas of endoderm differentiation, but no prostate formation. As another negative control, 17 grafts were generated from iEpt cells that were not infected by retroviruses expressing candidate MRs. Of these, 6 grafts formed teratomas, while an additional 11 grafts formed teratomas with areas of endodermal epithelial differentiation, characterized by formation of large ducts as well as tubular and glandular structures, but not prostate differentiation.

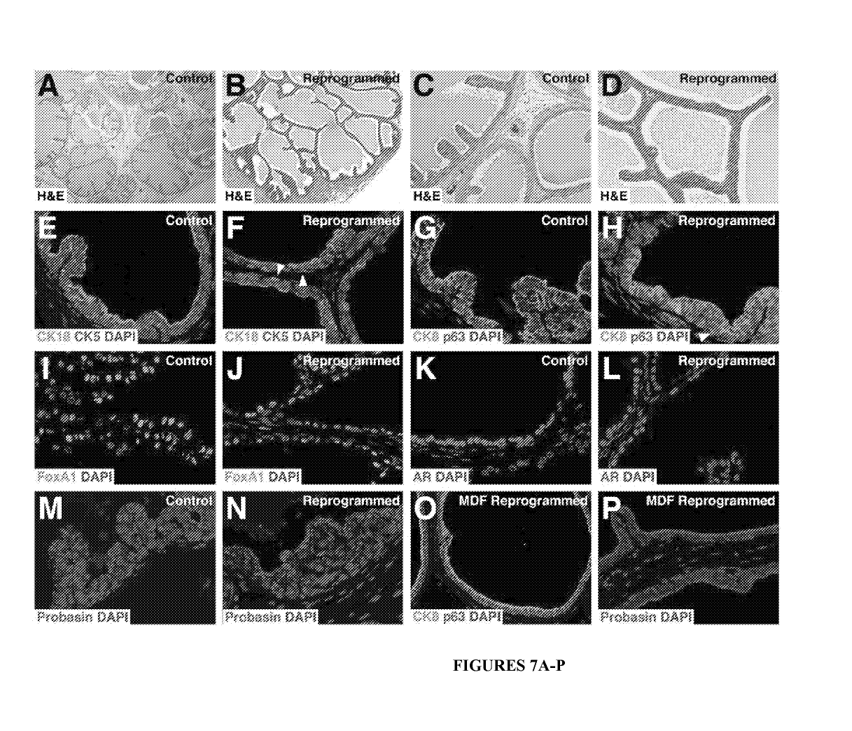

Overall, 13% (n=6/47) of the successful tissue grafts formed tissue structures that histologically resembled prostate tissue, as shown by hematoxylin-eosin staining of paraffin sections (FIG. 7A-D). Of the six successful grafts, five resulted from infection with a combination of AR and Nkx3.1 (3 grafts), or AR, Nkx3.1, and FoxA1 (2 grafts); only one successful graft grew from infection with a single candidate prostate MR (AR). Among the remaining grafts that grew from iEpt cells infected by candidate prostate MRs, 8 formed teratomas, while an additional 28 grafts formed teratomas with regions of endoderm epithelial differentiation, and an additional 6 grafts formed teratomas with apparent areas of prostate differentiation. These results indicate that the candidate MRs can be insufficient in these tissue recombinants to promote full prostate differentiation.

To confirm that the successful grafts reconstituted prostate tissue, immunostaining for specific markers of basal and luminal epithelial cells was performed. These marker analyses revealed a proper tissue architecture containing a basal epithelial layer expressing p63 and CK5, as well as a luminal epithelial layer expressing CK8, CK18, and AR (FIG. 7E-L). Luminal expression of probasin, a prostate-specific secretory protein, was also found, indicating that the reprogrammed prostate tissue was functional (FIG. 7M,N). Notably, iEpt cells formed from mouse dermal fibroblasts (MDFs) by transient doxycycline-regulated expression of an OSKM transgene can also be reprogrammed to form prostate tissue with proper expression of basal and luminal markers (FIG. 7O,P), with 9% (n=2/22) of the grafts generated from retroviral expression of AR and FoxA1 forming prostate tissue (and none with teratoma formation), indicating that iEpt cells generated by different methods can be reprogrammed successfully. Formation of prostate tissue in the direct conversion process is dependent on the expression of one or more prostate epithelial MRs, as well as the presence of embryonic urogenital mesenchyme.

Production of Human Prostate Tissue from Reprogrammed Fibroblasts by Tissue Recombination:

The ability of fibroblasts to generate human prostate tissue was investigated using a similar direct conversion approach. For this purpose, lentiviruses expressing doxycycline-inducible human OSKM was used together with the reverse tetracycline transactivator rtTA (Stemgent) to infect BJ normal human foreskin fibroblasts. Doxycycline was added at 2 days post-infection, and cells were cultured for 8 days in basal epithelial media, which resulted in approximately 15% frequency of conversion into iEpt cells. These human iEpt cells resembled the mouse iEpt cells in their expression of CK5, CK8, CK18, and beta-catenin (FIG. 6H). At this point, the human iEpt cells were transduced with human AR, FOXA1, and NKX3.1 retroviruses [A19, A54] in various combinations, followed by culture for an additional 10 days in the presence of doxycycline. At 20 days from the start of the experiment, these reprogrammed cells were recombined with rat embryonic urogenital mesenchyme and used for renal grafting, followed by harvesting after 8-10 weeks for analysis. This direct conversion protocol was highly efficient, since 69% (n=9/13) of the grafts grew exclusively as prostate tissue, while the remaining grafts did not grow at all.

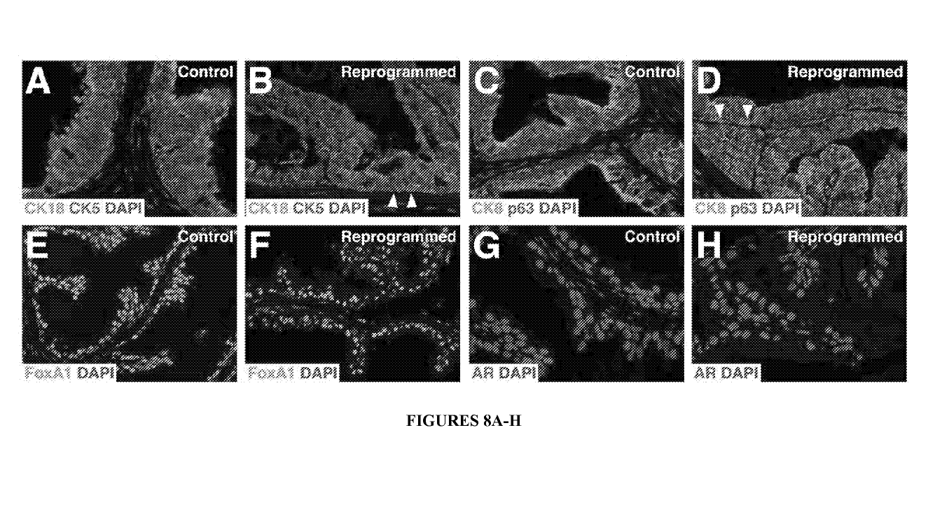

The resulting grafts were analyzed by H&E staining and immunostaining for specific epithelial markers, which showed their strong similarity to normal human prostate tissue (FIG. 8). Previous studies have reported that recombination of human prostate epithelium with rodent urogenital mesenchyme resulted in prostate tissue with human phenotypic characteristics, including a high basal/luminal ratio due to the presence of a continuous basal layer, unlike the mouse prostate [A55]. The reprogrammed human prostate tissue that was generated displayed a nearly continuous basal layer (FIG. 8B,D), unlike the reprogrammed mouse prostate (FIG. 6F,H), consistent with human tissue morphology.

The direct conversion process can be investigated using the optimization of direct conversion to prostate tissue using systems approaches to identify candidate master regulators for prostate epithelium. The mechanisms of direct conversion can be investigated, including analyses of potential intermediate pluripotent states, lineage-tracing of iEpt cells to identify potential progenitor cells, and molecular analyses of the reprogramming activity of urogenital mesenchyme. Direct conversion can be combined with gene targeting to establish genetically-engineered models of human prostate cancer.

Optimization of Direct Conversion into Prostate Epithelium:

Using candidate MRs identified by systems analyses, functional validation assays can be performed to identify successful reprogramming MR combinations for optimization of the direct conversion process. The quality of the reprogrammed mouse and human prostate tissue can be assessed using histopathological and molecular analyses. The efficiency of the reprogramming process can be assessed to determine the number of iEpt cells necessary for successful graft formation.

Experimental Design:

To determine whether candidate MRs can improve the reprogramming of iEpt cells in culture, lentiviral infection can be used to overexpress positive MRs or knock-down negative MRs in mouse and human iEpt cells, followed by tissue recombination and renal grafting. These experiments can be performed using synergistic combinations of candidate MRs identified bioinformatically, as well as using combinations of candidate MRs together with AR, Nkx3.1 and FoxA1, or individually as a control. If new MR combinations that appear to greatly enhance the efficiency or quality of direct conversion are identified, limiting dilution analyses can be performed as well as detailed marker studies of the reprogrammed prostate tissue.

For reprogrammed prostate tissues, H&E staining and immunostaining for specific markers can be performed (FIGS. 7, 8). In the case of reprogrammed human prostate tissues, the histological differences with mouse prostate can be assessed, including the basal/luminal ratio and the thickness of the stromal smooth muscle layer [A55]. Mouse prostate grafts can display similar morphologies at different time points, prostate grafts generated with human epithelial cells display a gradual time course of growth and differentiation over six months [A55]. The morphology of the reprogrammed human prostate tissue over time can be assessed by performing direct conversion and analyzing the resulting tissue at 1, 2, 4, and 6 months after grafting.

To assess the efficiency of direct conversion, limiting dilution analyses can be performed to determine the number of iEpt cells required for successful formation of prostate grafts. The number of urogenital mesenchyme cells remains constant at 250,000/graft, while the number of iEpt cells can be varied from 100 to 50,000. The results can then be analyzed by the extreme limiting dilution algorithm (ELDA) [A59], which has been used previously for analyses of graft formation by isolated prostate basal cells [A21]. In each experiment, the number of iEpt cells co-expressing prostate lineage master regulators can be determined retrospectively by immunostaining to adjust the cell numbers for the starting iEpt population.

Without being bound by theory, molecular analyses to investigate the similarity of reprogrammed prostate tissue to native mouse and human prostate tissue can be performed. Control mouse and human tissue grafts produced by tissue recombination of normal mouse and human prostate tissue with rat urogenital mesenchyme can also be analyzed. For example, expression profiles from at least six independent reprogrammed prostate grafts can be generated, as well as control grafts by RNA-sequencing. RNA-seq can then be performed using 30 million single-end reads generated on a high-throughput sequencing platform, such as the Illumina HiSeq 2000 platform. Expression profiles of normal adult mouse prostate tissue can be obtained by RNA-seq, while expression profiles of normal human prostate tissue can be obtained from publically available datasets [A57] and by RNA-seq analysis. The resulting expression profiles can be analyzed by Principal Components Analysis (PCA) and unsupervised hierarchical clustering to determine the overall similarity of these expression profiles [A21, A60]. Gene expression signatures of the reprogrammed tissue grafts versus normal control grafts can be generated to investigate their similarity to native mouse and human prostate tissue using Gene Set Enrichment Analysis (GSEA) [A21, A60].

Normal adult human prostate tissue can be obtained from primary cystectomy samples in which normal prostate tissue is surgically excised in conjunction with the removal of bladder tumors. The normal histology of the prostate tissue can be verified by pathological analysis.

In one embodiment, it is conceivable that these analyses can identify putative MR combinations that can promote direct conversion of fibroblasts to prostate tissue in the absence of transient expression of pluripotency factors. The properties of efficient reprogramming combinations can be investigated using alternative methods for direct conversion.

### Example 8

### Computational Systems Analysis for the Prediction of Master Regulators

An interactome for human prostate tissue has been generated, using the ARACNe algorithm for reverse engineering [A29, A30, A56]. This human prostate interactome was constructed from a large published dataset comprised of prostate cancer specimens and adjacent normal tissue [A57], and was validated by computational analysis of published genome-wide chromatin immunoprecipitation (ChIP) data for transcription factors such as c-Myc, AR, and BCL6, showing consistently high statistical significance.

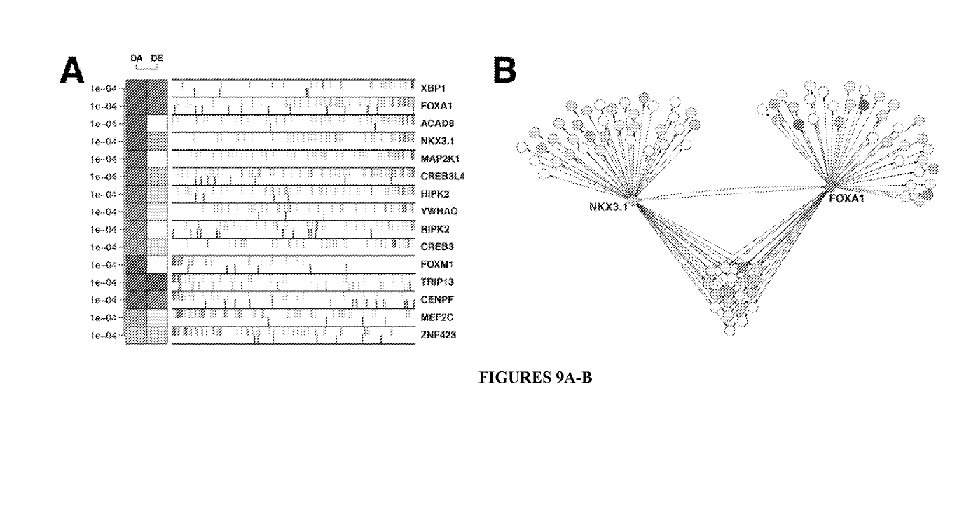

To identify master regulators (MRs) for normal prostate epithelium, the human prostate interactome was used for analysis using the MARINa algorithm [A32, A33]. Published gene expression profiles were used for mouse prostate tissue during organogenesis as well as adulthood [A58] to generate gene signatures for normal prostate tissue. Cross-species interrogation of the human prostate interactome using signatures for normal prostate differentiation during organogenesis (comparing embryonic to adult prostate) consistently identified both FoxA1 and Nkx3.1 among the top candidate MRs (FIG. 9A). The MARINa algorithm was used to identify synergistic pairs of MRs [A32, A33], which were defined as displaying a significantly stronger enrichment on the signature for co-regulated target genes than for the individually-regulated targets. FoxA1 and Nkx3.1 were computationally identified as a potential synergistic MR pair by this analysis (FIG. 9B). Without being bound by theory, these findings suggest that further computational systems analysis can identify additional candidate MRs for normal prostate epithelium as well as potential synergistic pairs to promote reprogramming to prostate tissue.

Successful reprogramming mouse and human fibroblasts into prostate tissue has been shown. A candidate gene approach has been used to identify putative master regulators (MRs) that promote direct conversion to prostate epithelium. A systems approach for the unbiased identification of such master regulators and their potential synergistic interactions can be used, and functional validation of the top candidate master regulators can be performed in the direct conversion assay. The direct conversion process can then be optimized by performing detailed histological and molecular analyses of the quality and efficiency of reprogramming by these MRs.

Experimental Design:

Published array data has been used for the identification of candidate MRs using the MARINa algorithm to interrogate the human prostate interactome, and has identified FOXA1 and NKX3.1, among others, as candidate MRs for prostate epithelium (FIG. 9). The outcomes of this algorithm are significantly more robust with expression signatures generated by RNA-sequencing. Compared to microarray platforms, RNA-seq analyses result in higher signal-to-noise ratio, display greatly enhanced transcript detection, and lack probe-derived bias.

To identify additional candidate MRs of prostate epithelium, gene expression profiling of adult mouse prostate tissue can be performed, as well as from embryonic (18.5 dpc) and neonatal (postnatal day 4 and day 12) prostate, with at least six samples for each time point. These tissues can be dissociated and used in flow cytometry using EpCAM antibodies to purify epithelial cells, followed by RNA-seq analysis. The resulting expression profiles can be used to generate signatures corresponding to embryonic, neonatal, and adult prostate epithelium. These expression signatures can be used to interrogate the human prostate interactome using the MARINa algorithm to identify candidate MR genes [A32, A33]; in parallel, similar analyses can be performed using a recently constructed mouse prostate interactome. Without being bound by theory, this approach can be used to identify potential synergistic pairs of candidate MRs [A32, A33].

Without being bound by theory, new candidate master regulators of prostate epithelium can be identified by these systems analyses. These candidate MRs can function synergistically with other prostate reprogramming factors to induce direct conversion to prostate epithelium. These system analyses can also identify negative MRs whose expression needs to be down-regulated to facilitate direct conversion; such reprogramming inhibitors are difficult to identify with candidate gene approaches. In one embodiment, candidate MRs can require co-expression in combination with several other reprogramming factors to induce prostate reprogramming.

### Example 9

### Analysis of Mechanisms of Direct Conversion to Prostate Epithelium

Without being bound by theory, the mechanisms of direct conversion to prostate epithelium can be analyzed by investigation of the steps of cellular reprogramming involved in the multi-step conversion process. For example, these studies can use lineage-tracing to identify the induced epithelial cell type(s) that are most amenable for reprogramming by prostate MRs, can examine whether successful reprogramming requires traversal through a transient pluripotent state, and can address the role of embryonic urogenital mesenchyme in promoting prostate transdifferentiation.

To understand the cellular and molecular mechanisms of direct conversion, the key features of the reprogramming process can be investigated. These studies can examine whether direct conversion proceeds through a pluripotent state, identify the cell type that gives rise to the prostate epithelial cells, and analyze the secreted factor(s) in the urogenital mesenchyme that is involved in prostate specification. These studies can provide important mechanistic insights into the reprogramming process.

Analysis of Traversal of the Pluripotent State:

Previous analyses of direct conversion protocols have concluded that the reprogramming process does not traverse a pluripotent state during the transdifferentiation process [A61-A63]. These analyses have not addressed the possibility that this pluripotent state may be extremely transient, and can only occur in a small percentage of the cell population that gives rise to the reprogrammed cells/tissue. Sporadic and transient expression of pluripotency markers in a small population of cells can be detected using a sensitive reporter. A mouse reagent that allows detection of Nanog expression, even if it occurs very transiently in a limited cell population has been developed.

Experimental Design:

Whether fibroblasts traverse the pluripotent state during generation of iEpt cells in culture can be investigated. MEFs from a mouse line carrying an IRES-GFP knock-in within the 3′ untranslated region of Oct4 [A64] can be generated. These Oct4-GFP MEFs can be used to determine whether rare GFP-positive cells can be identified during the formation of iEpt cells in basal medium. As a positive control, parallel cultures in mESC/LIF medium to generate iPSC colonies (GFP-positive) can be performed.

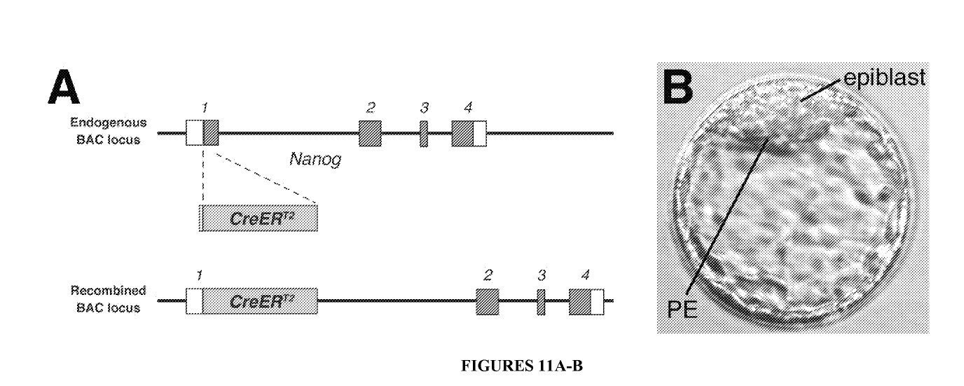

An inducible Nanog-CreER^{T2} transgene can be used in combination with the fluorescent Cre-reporter R26R-Tomato to perform lineage-marking of cells that express Nanog during direct conversion. MEFs containing the Nanog-CreER^{T2} transgene can only express the Tomato reporter if the Nanog promoter is activated by 4-hydroxy-tamoxifen (4-OHT), but continue to express Tomato even if Nanog is no longer expressed. (It is essential to use an inducible Cre driver under the control of the Nanog promoter, since a constitutively active Cre would promote Cre-reporter expression in pluripotent epiblast cells and thus all of the cells of the resulting mouse.) Two independent BAC (bacterial artificial chromosome) transgenic mouse lines that express CreER^{T2} under the control of the endogenous Nanog promoter (FIG. 11A) have been generated. To confirm that Cre-reporter expression recapitulates the expression pattern of Nanog, inducible lineage-marking of epiblast cells in Nanog-CreER^{T2}; R26R-Tomato/+ pre-implantation blastocysts has been successfully performed by administration of 4-hydroxy-tamoxifen (4-OHT) in culture (FIG. 11B).

MEFs from Nanog-CreER^{T2}; R26R-Tomato/+ mouse embryos can be generated, using the protocols that have been followed previously for MEF isolation and culture. The resulting MEFs can be utilized for the direct conversion protocol using doxycycline-inducible lentiviruses expressing human OSKM and rtTA for transient expression of pluripotency factors as described previously, but also cultured in the presence of 4-OHT. As a positive control, parallel reprogramming experiments can be performed using cell culture conditions that promote iPSC formation. Finally, if such traversal is observed, the contribution of Tomato-positive cells to the formation of reprogrammed prostate tissue can be investigated.

Without being bound by theory, Nanog-CreER^{T2} MEFs represent a sensitive reagent, since transient Nanog expression can be detected no matter when it occurs in the culture due to the indelible lineage-mark, and the level of Cre expression only needs to be sufficient to induce a single recombination event at the ROSA26 locus. Upon detection of Tomato expression in our cultures, the time point at which Cre-mediated recombination occurs can be identified, and the expression of Nanog and other pluripotency markers can be examined by quantitative RT-PCR and RNA-seq approaches. If reprogramming to prostate epithelium traverses a transient pluripotent state, as detected using the Nanog-CreER^{T2} mice, other direct conversion processes that have been reported in the literature can be investigated to determine whether a similar transient pluripotent state may occur.

Lineage-Tracing of the Cell of Origin for Converted Prostate Epithelium:

To determine whether the formation of reprogrammed prostate tissue in renal grafts recapitulates processes of normal organogenesis, or whether instead it mimics features of adult tissue homeostasis and/or regeneration, the cell type that gives rise to reprogrammed prostate epithelium can be investigated. During organogenesis, the basal epithelium contains progenitors for both basal and luminal cell types, whereas the luminal epithelium appears to be unipotent [A65]. In the adult prostate, bipotential progenitors exist in the basal epithelium during homeostasis and regeneration, but are relatively rare [A21], while luminal stem/progenitors have been identified during regeneration [A20]. Lineage-tracing of the iEpt cells in culture can be performed to determine which cell type(s) within this heterogeneous cell population can generate prostate epithelium in renal grafts. Specifically, inducible Cre drivers can be used to mark iEpt cells expressing basal or luminal markers to determine whether either or both cell populations can generate reprogrammed prostate epithelium in tissue recombinants. These studies can also be relevant for understanding the cell of origin for the human prostate tumors.

Experimental Design:

Lineage-tracing can be performed using inducible Cre drivers that mark basal or luminal subpopulations of the iEpt cells, which display heterogeneous marker phenotypes in culture (FIG. 6). To mark basal epithelial cells, the CK5-CreER^{T2} transgenic line that has been previously employed for lineage-tracing of prostate basal cells [A21] can be used. To mark luminal epithelial cells, the CK8-CreER^{T2} and CK18-CreER^{T2} transgenic lines that have been used for lineage-tracing of prostate epithelial cells during organogenesis [A65] can be used. Using these lines, MEFs from CK5-CreER^{T2}; R26R-YFP, CK8-CreER^{T2}; R26R-YFP, and CK18-CreER^{T2}; R26R-YFP mice can be generated. After generation of iEpt cells by infection with doxycycline-inducible OSKM lentiviruses, 4-OHT can be used to induce YFP expression in the corresponding CK5, CK8, or CK18 expressing iEpt population. The resulting lineage-marked iEpt population can then be isolated by flow-sorting, and used for lentiviral infection with prostate MRs and tissue recombination, followed by analysis of the resulting grafts to determine the distribution of YFP-expressing cells. Alternatively, the iEpt cells can be flow-sorted to isolate YFP-positive cells prior to prostate MR expression and tissue recombination, followed by analysis of grafts.

Without being bound by theory, if the reprogrammed prostate epithelium is derived from basal iEpt cells, lineage-tracing using the CK5-CreER^{T2} transgenic line would reveal extensive contribution of YFP-positive cells to the renal grafts. If luminal iEpt cells give rise to reprogrammed prostate tissue, lineage-tracing using the CK8-CreER^{T2} and CK18-CreER^{T2} mice would generate extensive YFP-positive contribution in the grafts. An interaction between basal and luminal iEpt cells can be necessary for generation of reprogrammed prostate tissue, which in this case would not be clonally derived. This interpretation would be suggested if flow-sorted basal and luminal iEpt cells are unable to form prostate tissue as purified populations, but can do so if mixed together prior to tissue recombination with urogenital mesenchyme. It may be the case that reprogrammed prostate tissue is generated from “intermediate” cells that co-express basal and luminal markers (such as CK5⁺CK8⁺ cells), which would be suggested if both purified populations of basal (CK5⁺) and luminal (CK8⁺) iEpt cells are able to generate prostate tissue. Further flow-sorting studies using cell-surface markers can be performed, such as the basal cell marker CD49f, in combination with CK8-CreER^{T2} lineage-tracing to isolate intermediate cells co-expressing basal and luminal markers. The ability of iEpt population(s) that generate reprogrammed prostate tissue to display stem cell properties, can be determined using assays that have been previously employed to identify stem cell populations in the adult prostate epithelium [A20, A21].

Systems Analysis of Embryonic Urogenital Mesenchyme:

Without being bound by theory, to identify the critical factor(s) responsible for the reprogramming properties of embryonic urogenital mesenchyme, a candidate pathway approach can be pursued, in combination with an unbiased systems analysis. For example, specific signaling pathways known to be active in embryonic urogenital mesenchyme can be tested for their necessity for reprogramming. Gene signatures of urogenital mesenchyme can be generated to interrogate the prostate interactomes.

Experimental Design:

In a candidate pathway approach, signaling pathways that have been implicated in prostate specification can be focused on, these include the canonical Wnt, FGF, and BMP pathways [A66]. To test whether these pathways are critical for prostate tissue reprogramming, lentiviral infection can be used to express secreted inhibitors of these pathways in mouse urogenital mesenchyme or to knock-down candidate signaling factors. For example, to test the role of canonical Wnt signaling, lentiviral overexpression of Dkk1 can be used to inhibit Wnt signaling, and as a control for its effects, the sensitive TCF/LefH2B-GFP transgenic reporter for canonical Wnt signaling activity [A67] can be used to monitor the consequences of Dkk1 overexpression. Similar approaches have been used to investigate the role of canonical Wnt signaling in early stages of prostate organogenesis [A51].

In the systems approach, differentially expressed genes as well as candidate master regulators can be identified. For this purpose, RNA-seq analyses can be performed to generate expression profiles of mouse embryonic urogenital mesenchyme as well as the neighboring bladder mesenchyme, which lacks reprogramming activity. Differentially expressed genes between urogenital mesenchyme and bladder mesenchyme can be identified, and gene ontology-biological process (GO-BP) analyses can be performed to identify differentially active signaling pathways. Expression signatures can be generated for urogenital mesenchyme to interrogate the mouse prostate interactome (which is based upon samples containing stromal tissue) for the identification of candidate MRs and synergistic MRs. These analyses can provide insights into signaling pathways and candidate ligands that can correspond to the reprogramming activity of the urogenital mesenchyme. Such candidate ligands can then be further investigated by lentiviral knock-down in the urogenital mesenchyme to determine whether their loss-of-function reduces or eliminates reprogramming activity.

For both approaches, if a candidate signaling ligand/pathway is identified as being critical for reprogramming activity using loss-of-function approaches, gain-of-function approaches to validate this finding can be used. Lentiviral infection can be performed to overexpress candidate ligands in rodent stromal cell lines that are derived from urogenital mesenchyme, but lack reprogramming activity, such as UGSM-2 [A68]. The resulting stromal cells can be investigated for its ability to support growth of normal prostate epithelium in tissue recombinants, as well as its ability to participate in direct conversion to prostate tissue.

Without being bound by theory, among the signaling pathways that have been investigated in prostate formation, there is evidence supporting a central role for canonical Wnt signaling [A51, A69-A71], and the candidate pathway approach can initially focus on canonical Wnt signaling. The reprogramming activity of urogenital mesenchyme can be at least partially unrelated to its inductive activity during prostate formation, and all candidate signaling pathways identified by systems analysis can be analyzed. In some embodiments, there can be cooperative effects and/or functional redundancy of multiple signaling factors that correspond to the reprogramming activity, analyses of synergistic MRs and GO biological processes can provide insights into the activities and identities of such cooperative signaling factors.

### Example 10

### Modeling of Human Prostate Cancer Initiation by Gene Targeting and Direct Conversion

An objective in stem cell biology is the development of therapies based on the generation of clinically relevant human cell types and tissues. In the context of disease, such approaches can also be harnessed for the creation of genetically engineered models of human cancer. Without being bound by theory, direct conversion/transdifferentiation methodologies can be employed to generate desired cell types and tissues from fibroblasts in culture, followed by their oncogenic transformation. In combination with gene targeting technologies, such approaches can be used to create precise genetically-engineered models of human cancer.

Despite the widespread use of mouse models of cancer, such models can be limited by their inability to fully recapitulate the physiological processes underlying human cancer, and can be limited for applications such as preclinical testing of candidate therapeutics. For example, analogous mouse and human tissues can have important anatomical and/or physiological differences, such as the strictly ductal histology of the mouse prostate gland versus the ductal-acinar structure of the human prostate. Consequently, it is essential to develop model systems using human tissue that can accurately recapitulate cancer, yet are amenable to gene targeting approaches and other genetic manipulations.

Without being bound by theory, cellular reprogramming methods can be used to develop a new generation of models of human cancer, using prostate cancer as a model system. For example, the direct conversion of mouse and human fibroblasts into prostate epithelium together with tissue recombination approaches can be used to generate histologically normal prostate tissue in renal grafts. In combination with gene targeting of tumor suppressors using Transcription Activator-Like Effector nucleases (TALENs), this approach can generate oncogenically transformed prostate tissue, which can have considerable clinical relevance for the generation of prostate cancer models.

Human prostate cancer initiation can be modeled by gene targeting and direct conversion using TALENs for the specific alteration of tumor suppressor genes that are mutated in human prostate cancer, followed by the generation of prostate tissue using the direct conversion methodology. Histopathological and molecular analysis of the resulting transformed prostate tissue can allow functional analysis of the roles of these tumor suppressors in human prostate cancer initiation and progression.

Without being bound by theory, these studies can provide the basis for an approach to human cancer modeling, which can lead to new insights into the molecular basis of human cancer initiation and progression as well as improved pre-clinical studies of candidate therapeutics.

TALEN-Mediated Gene Targeting in Human Fibroblasts and Prostate Epithelial Cells:

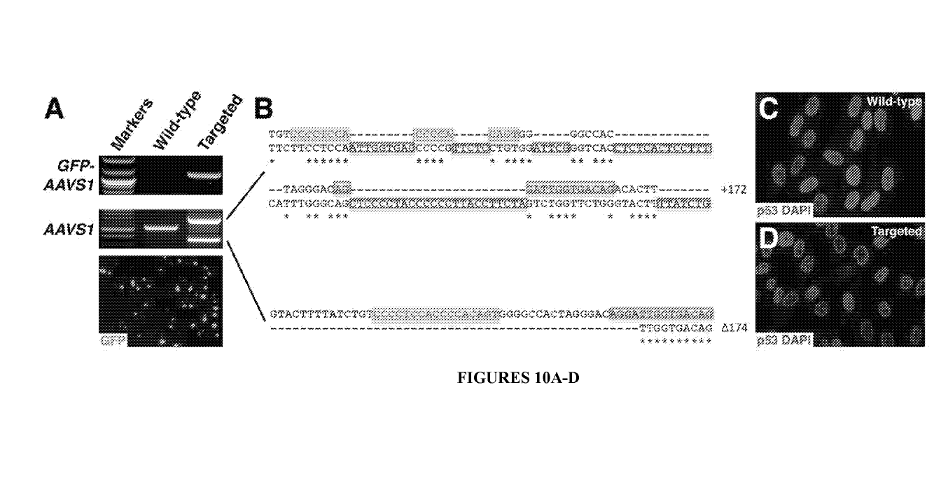

To demonstrate the feasibility of gene targeting in combination with direct conversion, TALENs have been used for gene targeting in the RWPE-1 human prostate epithelial cell line as well as in BJ foreskin fibroblasts. AAVS1, which encodes the PPR1R12C gene has been targeted and is a well-characterized locus used previously for gene targeting in human embryonic stem cells [A37]. Using published TALEN pairs and a GFP-expressing puromycin-resistance donor cassette [A37], AAVS1 was successfully targeted in both cell lines. To eliminate non-specific targeting, the cells were selected in puromycin followed by clonal growth by limiting dilution. Analysis of the AAVS1 locus showed proper targeting and integration of the donor GFP cassette (FIG. 10A). Sequence analysis showed that both AAVS1 alleles were mutated in the clones analyzed, indicating the high efficiency of targeting (FIG. 10B). TALENs have been used to target the TP53 locus in human BJ fibroblasts. Analyses are consistent with efficient targeting, as p53 expression is not up-regulated following adriamycin treatment, in comparison with control fibroblasts (FIG. 10C,D).

To generate genetically-engineered models of human prostate cancer initiation and early progression, gene targeting using TALE nucleases can be performed in human fibroblasts followed by direct conversion into prostate tissue. Straightforward targeting mediated by non-homologous end joining to generate loss-of-function alleles, or a two-step homologous recombination approach to create specific point mutations, can be used. These studies can permit the analysis of early events in cancer initiation in human prostate, which has previously been inaccessible to molecular genetic analysis.

Experimental design: Gene targeting of PTEN and TP53 in human fibroblasts can be performed. These tumor suppressors have been selected since their loss-of-function can yield prostate cancer phenotypes. Notably, in mouse models, loss of PTEN function results in high-grade PIN and eventually adenocarcinoma [A72-A75], while TP53 loss does not have a cancer phenotype, but deletion of both genes results in aggressive adenocarcinoma [A76]. To introduce deletions at the start codon of these two genes, published TALENs (Addgene) that cleave near the N-terminus of the protein coding sequence [A38] can be used. Targeting of PTEN and TP53 in human BJ fibroblasts can be performed, followed by the direct conversion protocol to form prostate tissue in renal grafts using immunodeficient NCR nude mice. These studies can be performed using targeting of PTEN or TP53 individually, or can use sequential targeting of both tumor suppressors. The resulting tissue grafts can be analyzed histologically for a PIN and/or adenocarcinoma phenotype. Basal (p63, CK5, CK14) and luminal (CK8, CK18) markers can be analyzed to ascertain whether the PIN/tumor lesions have a strong luminal phenotype that is typical of human prostate adenocarcinoma. The expression of alpha-methylacyl-CoA racemase (AMACR), which is up-regulated in human prostate cancer [A77], can be assessed. If robust tumor formation is observed, these tumors can then be propagated by renal or orthotopic grafting in immunodeficient mice.

The creation of a specific point mutation in TP53 can be performed, using an approach similar to that employed for genetic-engineering in mouse ES cells. TALENs can mediate gene targeting in human cells by homologous recombination with insertion vectors, analogous to conventional approaches in mouse ES cells, including two-step procedures that can introduce point mutations followed by Cre-loxP recombination to remove inserted drug-selection cassettes [A37]. These studies can use a two-step targeting approach to introduce a specific missense mutation, R273H, into the TP53 coding region in fibroblast cells that are either wild-type or contain a homozygous PTEN null mutation, followed by phenotypic analysis of reprogrammed prostate tissue. The TP53 residue 8273 is a mutational hotspot in human cancer, including prostate cancer [A78]. Studies in genetically engineered mice show that the corresponding Tp53^{R27OH} mutation has a prostate cancer phenotype distinct from that of Tp53 null mutants, suggesting a potential role for TP53 in prostate cancer initiation rather than in advanced disease [A79].

The creation of mutations in genes that have recently been identified in whole-genome and exome sequencing projects as mutated in human prostate cancer can be performed. Although human prostate cancer displays a relatively low mutation rate in general, particularly for many known tumor suppressor genes, a significant number of genes have been found to be mutated that have not been functionally characterized to any significant degree, including genes such as SPOP, MED12, and HOXB13 [A57, A78, A80-A83]. To address the functional significance of these genes in human prostate cancer progression, these genes can be mutated either individually or in combination with PTEN or other tumor suppressors in human fibroblasts to investigate the phenotype of the resulting reprogrammed prostate tissue. TALENs can be created to mutate the desired target sites using currently available reagents (Addgene) [A38], and use non-homologous end joining to mutate genes to create simple loss-of-function alleles (e.g., for SPOP mutations) or homologous recombination to create specific point mutations (e.g., for the HOXB13 G48E allele).

Without being bound by theory, these studies can provide the foundation for new genetically-engineered models of human prostate cancer. Studies of the cell of origin of reprogrammed prostate tissue can be relevant for understanding the cell of origin for prostate cancer, which can originate either from luminal or basal cells in mouse models [A21, A84]. In some embodiments, there may be intrinsic variability in the extent of reprogramming that can complicate the interpretation of tumor phenotype. Continued development of the TALEN technology can undoubtedly lead to its application for chromosomal engineering, as is now commonly performed using Cre-loxP technology [A85], and allow for the recapitulation of the extensive genomic rearrangements that typically take place in prostate cancer, such as the frequent TMPRSS2-ERG gene fusion. In other embodiments, targeting of certain tumor suppressor genes may affect the efficiency and possibly the outcome of direct conversion, since reduced function of the p53-p21 pathway greatly increases efficiency of fibroblast reprogramming to iPSC [A86-A89]. The generation of human prostate tumor models using TALEN-mediated gene targeting, allows for future studies that can extend the applicability of this approach. Chromosomal engineering approaches can be used to generate the TMPRSS2-ERG fusion and other genomic rearrangements in reprogrammed prostate tumors. The molecular mechanisms of castration-resistance in this system can also be investigated, including the possibility of endogenous androgen biosynthesis by reprogrammed tumors.

Without being bound by theory, the direct conversion/transdifferentiation to prostate epithelium can provide the basis for many future studies of reprogramming. In particular, the approaches developed herein can be generally applicable for reprogramming to other tissues of interest, and for creating genetically-engineered models for a range of human cancers. The systems analyses coupled with mechanistic and functional studies can yield insights into normal processes of prostate organogenesis and stem cell biology. The use of xenograft-based genetically-engineered models of human cancer permits the extension to analyses of candidate therapeutics and drug response.

### Example 11

### Production of Mouse Prostate Tissue from Reprogrammed Fibroblasts by Tissue Recombination and Lentiviral Expression of Prostate Master Regulators

Doxycycline-inducible lentiviral pluripotency factors, OSKM, were used to reprogram mouse embryonic fibroblasts (MEFs) to induced epithelial (iEpt) cells in culture. This allows precise timing of expression of the pluripotency factors, OSKM. Lentiviruses were produced in 293FT packaging cells using established protocols. Lentiviruses were pooled and filtered prior to infection. 2 days after infection, MEFs were treated with Dox for 7-9 days to induce the pluripotency factors in 10% FBS/DMEM or 10% KSR/DMEM, no LIF was added to the media. After 7-9 days, Dox was withdrawn from the media and cells were infected with lentiviruses expressing human NKX3.1 (pLOC NKX3.1 iresGFP), human AR (pLentiV6.2 HA-AR), and human FOXA1 (pSIN-EF2 Foxa1-puro) (NAF cocktail) and cultured in prostate basal media (Cnt-12, Cnt-Prime media, CellnTEC) for 7 days. To avoid confusion with host derived cells, prior to tissue recombination, an additional infection with pLOC RFP lentiviruses was performed to color-mark the iEpt-NAF cells.

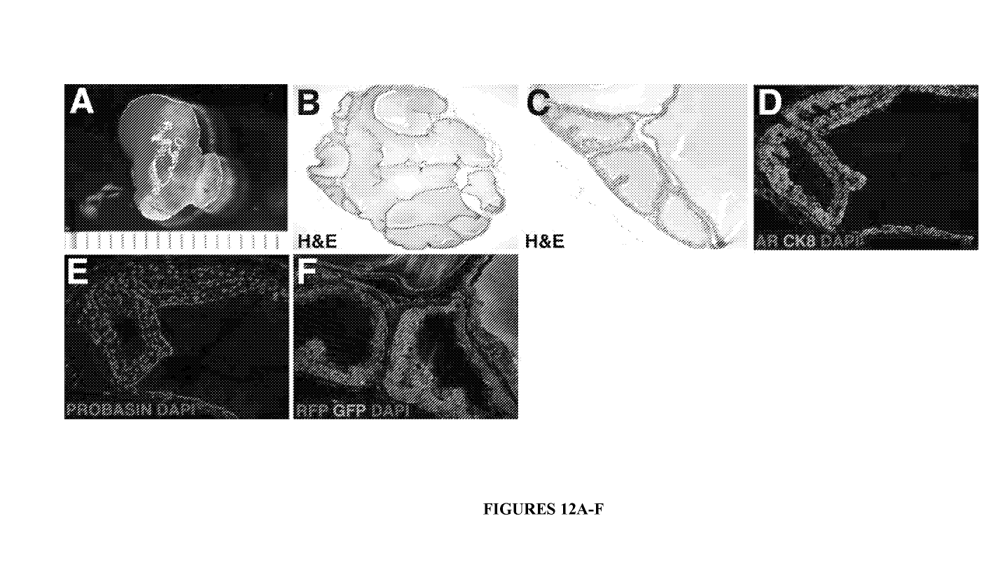

In the next step, the iEpt-NAF cells were recombined with rat embryonic urogenital sinus mesenchyme (UGM) and grafted under the renal capsule of athymic nude mice. The tissue recombinants were harvested after 6-8 weeks and analyzed by hematoxylin-eosin staining and immunostaining for prostate tissue specific markers. Similar to our experimental set-up, this combination of transient expression of lentiviral pluripotency factors and lentiviral transduced master regulators of prostate development were able to reprogram MEFs to iEpt cells which were able to grow into prostate tissue under the inductive force of UGM (FIG. 12A-C). The induced prostate tissue expresses AR (FIG. 12D) and it is functional as shown by immunostaining with Probasin, a prostate secretion specific marker (FIG. 12E). We confirmed that the induced tissue was indeed generated from our reprogrammed iEpt cells by positive immunostaining for GFP (from hNKX3.1 ires GFP vector) and RFP (from the pLOC RFP infections).

### Example 12

### Production of Mouse Bladder Tissue from Reprogrammed Fibroblasts by Tissue Recombination

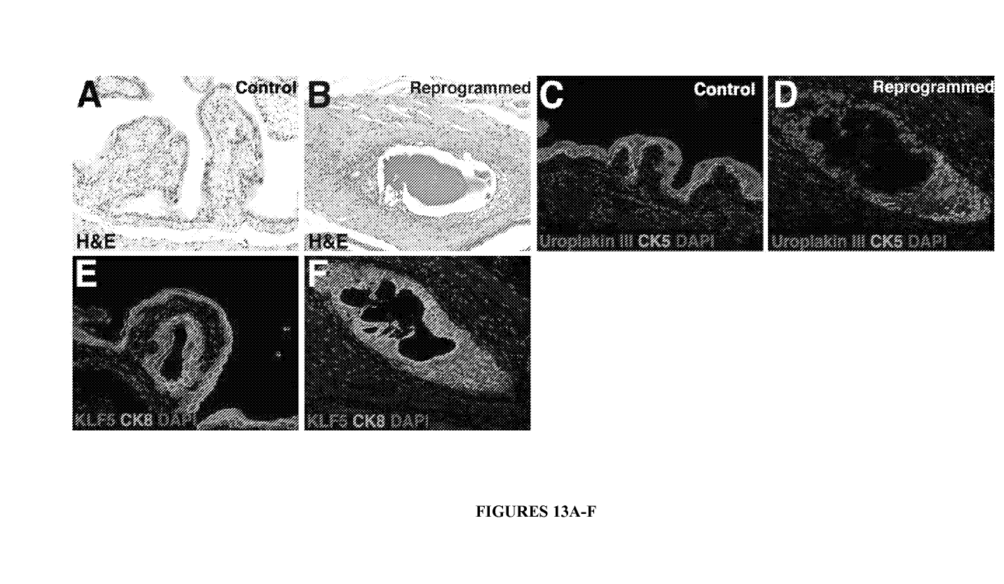

KLF5 has been used as a master regulator of bladder development [B1] to re-specify iEpt cells towards bladder epithelia in tissue recombination experiments with rat embryonic bladder mesenchyme. When KLF5 is missing from the bladder epithelial cells, urothelial precursor cells remain in an undifferentiated state and the resulting urothelium fails to stratify and to express terminal differentiation markers (e.g. uroplakins). Similar to the reprogramming to prostate tissue experiments, we have used KLF5 expressing lentiviruses to infect iEpt cells. iEpt-KLF5 cells were further recombined with rat embryonic bladder mesenchyme and grafted under the renal capsule. In this set-up, 4/4 renal grafts grew (FIG. 13B) and contained uroplakin-positive areas (FIG. 13D) similar to WT bladder tissue (FIG. 13C). In addition, the reprogrammed uroplakin-positive areas shown a proper distribution of the CK5 and CK8 epithelial layers and were positive for KLF5 (FIG. 13C-F).

### Example 13

### Production of Mouse Bladder and Prostate Tissue from iPS

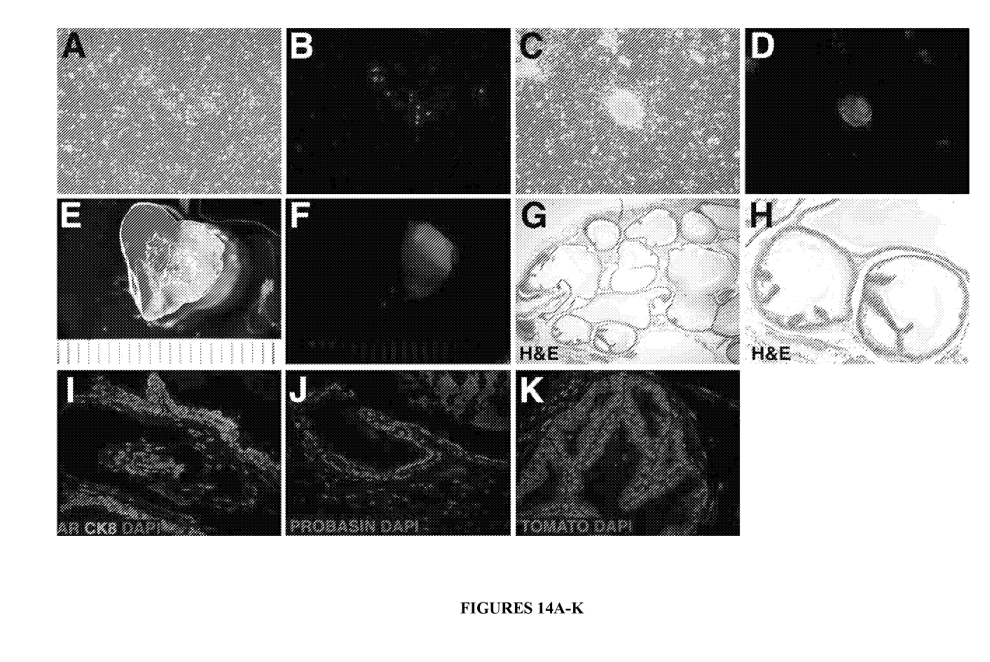

The same doxycycline-inducible pluripotency factors, OSKM, were used to reprogram MEFs from CK18CreERT2/Rosa26-Tomato to induced pluripotent cells (iPS) cells in culture. Cells of the above genotypes were infected with OSKM and rtTA lentiviruses and cultured in mouse embryonic stem cell media in the presence of LIF. According to iPS published protocols, Dox was added to the media for 11 days to induce the pluripotency factors, followed by Dox-free media for another 5-7 days when iPS colonies were picked and moved on a mitomycin-treated fibroblast feeder layer. 1 μM 4-hydroxy Tamoxifen (4-OHT, (Z)-4-Hydroxytamoxifen, H7904, Sigma) was also added to the media after the OSKM infection until the iPS colonies picking to lineage-trace cells which expressed CK18 or Gata6. In accord with previous literature, upon OSKM activation, a proportion of the MEFs undergo a transition to an CK18+ epithelial phenotype and express Tomato in the presence of 4-OHT (FIG. 14A,B). Some of these Tomato-positive cells developed into iPS colonies after 11 days of Dox induction (FIG. 14C,D). A single Tomato-positive iPS colony was picked from the plate at Day 12 and recombined undissociated with rat UGM in collagen. The resulting cell recombinant was grafted under the renal capsule of an athymic nude mouse. The renal graft was harvested at 8 weeks post-grafting and analyzed by gross microscopy (FIG. 14 E,F), H&E for histology (FIG. 14G,H) and by immunostaining for epithelial (CK8) and prostate specific markers (AR, Probasin) (FIG. 14I,J). The resulting graft was Tomato-positive (FIG. 14 F,K) demonstrating that it originated from the CK18CreERT2/R26r-Tomato iPS colony and had histology and tissue specific markers similar to native prostate tissue.

A similar strategy can be employed to generate bladder tissue from a single iPS colony after recombination with rat embryonic bladder mesenchyme.

### Example 14

### Production of Mouse Bladder and Prostate Tissue from iPS-Derived Endodermal Cells

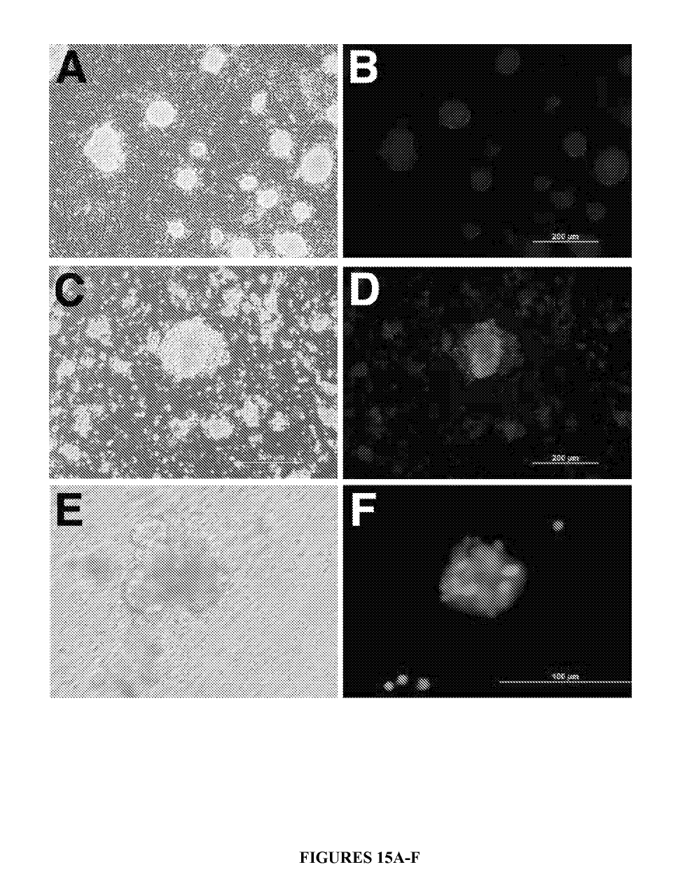

Using the same Dox-inducible reprogramming protocol, iPS cells were generated from Gata6CreERT2/Rosa26-caggEYFP MEFs. Passaged 2 iPS colonies (FIG. 15A,B) (4 independent colonies) were replated on 0.1% gelatin coated plates and the mES media was changed to endodermal differentiation media containing Activin A (50 ng/ml; RnD Systems, Minneapolis, USA), Noggin (200 ng/ml; RnD Systems) and a GSK3β inhibitor (1 μM of 6-bromo indirubin-3-oxine, BIO; Merck KGaA, Darmstadt, Germany) in 25% F-12/75% IMDM/2 mM Glutamax/0.55 mM beta-mercaptoethanol/N2 supplement [2]. 4-OHT was added to the differentiation media to mark endodermal differentiated cells. Numerous YFP+ colonies were observed at 4-6 days of culturing in this media indicating that these cells express or passed through a GATA6-positive state (FIG. 15C,D). The YFP+ cells were sorted after 6 days of differentiation and analyzed for expression of endodermal markers by RT-PCR. As expected, these cells expressed GATA6 and SOX7 mRNA at high levels compared with MEFs. For the differentiation towards prostate and bladder lineages, YFP+ endodermal cells were plated in 3D-culture conditions in matrigel with (for prostate) or without (for bladder) dihydrotestosterone propionate (DHT, Sigma). In these culture conditions, spherical growth of some of the YFP+ cells was observed (FIG. 15E,F). These endodermal 3D-structures can be grafted under the renal capsule of nude mice after recombination with rat embryonic UGM or bladder mesenchyme.

### Example 15

### Protocol for Direct Transdifferentiation of Mouse Fibroblasts to Induced Prostate and Bladder Tissue Using Lentiviral Vectors

As an alternative to continuous activation of the pluripotency factors, our reprogramming protocols were switched to a lentiviral OSKM cocktail. Specifically, doxycycline-inducible lentiviral vectors expressing the pluripotency factors, Oct4, Sox2, KLF4 and cMyc together with the vector expressing the reverse tetracycline transactivator (rtTA) were acquired from Addgene (FU-tet-o-hOct4, cat.no 19778; FU-tet-o-hSox2, cat.no 19779; FU-tet-o-hKLF4, cat.no 19777; FU-tet-o-hc-myc, cat.no 19775; FUdeltaGW-rtTA, cat.no 19780). Lentiviruses were produced in 293FT packaging cells using established protocols for second generation lentiviral system based on the packaging plasmids pMD2.G (VSV-G envelope expressing plasmid, cat. no 12259) and psPAX2 (Addgene cat. no 12260). Briefly, 293FT cells were transfected with the packaging plasmids and the OSKM and rtTA encoding plasmids using Lipofectamine 2000 (Invitrogen, cat.no 11668-019). Each lentivirus was produced separately. Lentiviruses were collected at 48 hrs and 72 hrs post-transfection, pooled and filtered prior to infection. Thus, mouse embryonic fibroblasts derived from WT 129Sv mice, Oct4-GFP knock-in, Nkx3.1 Lacz+/−, CK18CreERT2/Rosa26-Tomato, Gata6CreERT2/Rosa26-caggEYFP mice were infected twice at 6 hours interval with a pool of lentiviruses encoding OSKM and rtTA. 48 hours after the last infection, MEFs cultured in 10% FBS/DMEM or 10% KSR/DMEM (FBS from Gemini, KSR and DMEM from Invitrogen) were treated with doxycycline (Dox) for 7-9 days to induce the pluripotency factors OSKM.

For generation of prostate tissue: After 7-9 days, Dox was withdrawn from the media and induced epithelial cells (iEpt) cells were infected twice at 6 hrs interval with lentiviruses expressing human NKX3.1 (pLOC NKX3.1 iresGFP; human AR (pLentiV6.2 HA-AR), and human FOXA1 (pSIN-EF2 Foxa1-puro) (NAF cocktail). The lentiviruses were produced in 293FT cells using the same packaging plasmid system as above. After the last NAF infection, the cell media was switched to prostate basal epithelial media (Cnt-12, CellnTEC) or generic basal epithelial media (Cnt-Prime media, CellnTEC) for 7 days. In some experiments, to avoid confusion with host-derived cells, prior to tissue recombination, an additional infection with pLOC RFP lentiviruses (derived from the pLOC RFP ires GFP vector obtained from the Califano Lab by removing the ires GFP cassette) was performed to color-mark the iEpt-NAF cells.

For generation of bladder tissue: After 7-9 days, Dox was withdrawn from the media and induced epithelial cells (iEpt) cells were infected twice at 6 hrs interval with lentiviruses expressing human KLF5 (pSIN-EF2 KLF5-puro). The KLF5 lentiviruses were produced in 293FT cells using the same packaging plasmid system as above. After the last KLF5 infection, the cell media was switched to generic basal epithelial media (Cnt-Prime media, CellnTEC) for 7 days. In some experiments, to avoid confusion with host-derived cells, prior to tissue recombination, an additional infection with pLOC RFP lentiviruses was performed to color-mark the iEpt-KLF5 cells.

In the next step, the iEpt-NAF and iEpt-KLF5 cells were recombined with rat embryonic urogenital sinus mesenchyme (UGM) and rat embryonic bladder mesenchyme, respectively in collagen. The recombined cells in collagen were grafted under the renal capsule of athymic nude mice. The tissue recombinants were harvested after 6-8 weeks and analyzed by hematoxylin-eosin staining and immunostaining for epithelial (CK5, CK8, CK18); endodermal (Foxa1, KLF5); prostate tissue specific (AR, Probasin) or bladder specific markers (Uroplakin III). The cultured origin of the tissues in the grafts was verified by GFP (for Nkx3.1 ires GFP) and RFP (for pLOC RFP) immunostaining.

Two further new approaches to generate prostate and bladder epithelial tissues in vivo are described. In the first instance, prostate tissue was generated from CK18CREert2/R26r-Tomato iPS after recombination with rat embryonic UGM. In the second instance, endodermal differentiation experiments with Gata6CreERT2/R26r-caggYFP iPS were performed. The endodermal cells can be recombined with tissue specific mesenchyme and renal grafted.

### REFERENCES

1) Efe, J. A., Hilcove, S., Kim, J., Zhou, H., Ouyang, K., Wang, G., Chen, J. and Ding, S. (2011). Conversion of mouse fibroblasts into cardiomyocytes using a direct reprogramming strategy. Nature cell biology 13, 215-222.
2) Kim, J., Efe, J. A., Zhu, S., Talantova, M., Yuan, X., Wang, S., Lipton, S. A., Zhang, K. and Ding, S. (2011). Direct reprogramming of mouse fibroblasts to neural progenitors. Proceedings of the National Academy of Sciences of the United States of America 108, 7838-7843.
3) Bell, S. M., Zhang, L., Mendell, A., Xu, Y., Haitchi, H. M., Lessard, J. L. and Whitsett, J. A. (2011). Kruppel-like factor 5 is required for formation and differentiation of the bladder urothelium. Developmental biology
4) Wang, X., Kruithof-de Julio, M., Economides, K. D., Walker, D., Yu, H., Halili, M. V., Hu, Y.-P., Price, S. M., Abate-Shen, C. and Shen, M. M. (2009). A luminal epithelial stem cell that is a cell of origin for prostate cancer. Nature 461, 495-500.
5) Taylor, R. A., Cowin, P. A., Cunha, G. R., Pera, M., Trounson, A. O., Pedersen, J. and Risbridger, G. P. (2006). Formation of human prostate tissue from embryonic stem cells. Nat Methods 3, 179-181.
6) Cunha, G. R., Fujii, H., Neubauer, B. L., Shannon, J. M., Sawyer, L. and Reese, B. A. (1983). Epithelial-mesenchymal interactions in prostatic development. I. morphological observations of prostatic induction by urogenital sinus mesenchyme in epithelium of the adult rodent urinary bladder. The Journal of cell biology 96, 1662-1670.
7) Baskin, L. S., Hayward, S. W., Sutherland, R. A., DiSandro, M. J., Thomson, A. A., Goodman, J. and Cunha, G. R. (1996). Mesenchymal-epithelial interactions in the bladder. World journal of urology 14, 301-309.
8) Baskin, L. S., Hayward, S. W., Young, P. and Cunha, G. R. (1996). Role of mesenchymal-epithelial interactions in normal bladder development. The Journal of urology 156, 1820-1827.
9) DiSandro, M. J., Li, Y., Baskin, L. S., Hayward, S. and Cunha, G. (1998). Mesenchymal-epithelial interactions in bladder smooth muscle development: epithelial specificity. The Journal of urology 160, 1040-1046; discussion 1079.
10) Liu, W., Li, Y., Cunha, S., Hayward, G. and Baskin, L. (2000). Diffusable growth factors induce bladder smooth muscle differentiation. In vitro cellular & developmental biology. Animal 36, 476-484.
11) Oottamasathien, S., Wang, Y., Williams, K., Franco, O. E., Wills, M. L., Thomas, J. C., Saba, K., Sharif-Afshar, A. R., Makari, J. H., Bhowmick, N. A., DeMarco, R. T., Hipkens, S., Magnuson, M., Brock, J. W., 3rd, Hayward, S. W., Pope, J. C. t. and Matusik, R. J. (2007). Directed differentiation of embryonic stem cells into bladder tissue. Dev Biol 304, 556-566.
12) Baskin, L. S., Hayward, S. W., Young, P. and Cunha, G. R. (1996). Role of mesenchymal-epithelial interactions in normal bladder development. J Urol 156, 1820-1827.
13) Anumanthan, G., Makari, J. H., Honea, L., Thomas, J. C., Wills, M. L., Bhowmick, N. A., Adams, M. C., Hayward, S. W., Matusik, R. J., Brock, J. W., 3rd and Pope, J. C. t. (2008). Directed differentiation of bone marrow derived mesenchymal stem cells into bladder urothelium. J Urol 180, 1778-1783.
14) Neubauer, B. L., Chung, L. W., McCormick, K. A., Taguchi, 0., Thompson, T. C. and Cunha, G. R. (1983). Epithelial-mesenchymal interactions in prostatic development. II. Biochemical observations of prostatic induction by urogenital sinus mesenchyme in epithelium of the adult rodent urinary bladder. The Journal of cell biology 96, 1671-1676.
15) Margolin, A. A., Wang, K., Lim, W. K., Kustagi, M., Nemenman, I. and Califano, A. (2006). Reverse engineering cellular networks. Nat Protoc 1, 662-671.
16) Margolin, A. A., Nemenman, I., Basso, K., Wiggins, C., Stolovitzky, G., Dalla Favera, R. and Califano, A. (2006). ARACNE: an algorithm for the reconstruction of gene regulatory networks in a mammalian cellular context. BMC Bioinformatics 7 Suppl 1, S7.
17) Basso, K., Margolin, A. A., Stolovitzky, G., Klein, U., Dalla-Favera, R. and Califano, A. (2005). Reverse engineering of regulatory networks in human B cells. Nat Genet 37, 382-390.
18) Lefebvre, C., Rajbhandari, P., Alvarez, M. J., Bandaru, P., Lim, W. K., Sato, M., Wang, K., Sumazin, P., Kustagi, M., Bisikirska, B. C., Basso, K., Beltrao, P., Krogan, N., Gautier, J., Dalla-Favera, R. and Califano, A. (2010). A human B-cell interactome identifies MYB and FOXM1 as master regulators of proliferation in germinal centers. Mol Syst Biol 6, 377.
19) Carro, M. S., Lim, W. K., Alvarez, M. J., Bollo, R. J., Zhao, X., Snyder, E. Y., Sulman, E. P., Anne, S. L., Doetsch, F., Colman, H., Lasorella, A., Aldape, K., Califano, A. and Iavarone, A. (2010). The transcriptional network for mesenchymal transformation of brain tumours. Nature 463, 318-325.
20) Zhou, Q., Brown, J., Kanarek, A., Rajagopal, J. and Melton, D. A. (2008). In vivo reprogramming of adult pancreatic exocrine cells to beta-cells. Nature 455, 627-632.
21) Ieda, M., Fu, J. D., Delgado-Olguin, P., Vedantham, V., Hayashi, Y., Bruneau, B. G. and Srivastava, D. (2010). Direct reprogramming of fibroblasts into functional cardiomyocytes by defined factors. Cell 142, 375-386.
22) Vierbuchen, T., Ostermeier, A., Pang, Z. P., Kokubu, Y., Sudhof, T. C. and Wernig, M. (2010). Direct conversion of fibroblasts to functional neurons by defined factors. Nature 463, 1035-1041.
23) Pang, Z. P., Yang, N., Vierbuchen, T., Ostermeier, A., Fuentes, D. R., Yang, T. Q., Citri, A., Sebastiano, V., Marro, S., Sudhof, T. C. and Wernig, M. (2011). Induction of human neuronal cells by defined transcription factors. Nature
24) Szabo, E., Rampalli, S., Risueno, R. M., Schnerch, A., Mitchell, R., Fiebig-Comyn, A., Levadoux-Martin, M. and Bhatia, M. (2010). Direct conversion of human fibroblasts to multilineage blood progenitors. Nature 468, 521-526.
25) Huang, P., He, Z., Ji, S., Sun, H., Xiang, D., Liu, C., Hu, Y., Wang, X. and Hui, L. (2011). Induction of functional hepatocyte-like cells from mouse fibroblasts by defined factors. Nature 475, 386-389.
26) Sekiya, S. and Suzuki, A. (2011). Direct conversion of mouse fibroblasts to hepatocyte-like cells by defined factors. Nature 475, 390-393.
27) Efe, J. A., Hilcove, S., Kim, J., Zhou, H., Ouyang, K., Wang, G., Chen, J. and Ding, S. (2011). Conversion of mouse fibroblasts into cardiomyocytes using a direct reprogramming strategy. Nat Cell Biol 13, 215-222.
28) Kim, J., Efe, J. A., Zhu, S., Talantova, M., Yuan, X., Wang, S., Lipton, S. A., Zhang, K. and Ding, S. (2011). Direct reprogramming of mouse fibroblasts to neural progenitors. Proc Natl Acad Sci USA 108, 7838-7843.
29) Wang, X., Kruithof-de Julio, M., Economides, K. D., Walker, D., Yu, H., Halili, M. V., Hu, Y. P., Price, S. M., Abate-Shen, C. and Shen, M. M. (2009). A luminal epithelial stem cell that is a cell of origin for prostate cancer. Nature 461, 495-500.
30) Cunha, G. R., Chung, L. W., Shannon, J. M., Taguchi, 0. and Fujii, H. (1983). Hormone-induced morphogenesis and growth: role of mesenchymal-epithelial interactions. Recent progress in hormone research 39, 559-598.
31) Niu, Y., Wang, J., Shang, Z., Huang, S. P., Shyr, C. R., Yeh, S. and Chang, C. (2011). Increased CK5/CK8-positive intermediate cells with stromal smooth muscle cell atrophy in the mice lacking prostate epithelial androgen receptor. PloS one 6, e20202.
32) Bhatia-Gaur, R., Donjacour, A. A., Sciavolino, P. J., Kim, M., Desai, N., Young, P., Norton, C. R., Gridley, T., Cardiff, R. D., Cunha, G. R., Abate-Shen, C. and Shen, M. M. (1999). Roles for Nkx3.1 in prostate development and cancer. Genes & development 13, 966-977.
33) Gao, N., Ishii, K., Mirosevich, J., Kuwajima, S., Oppenheimer, S. R., Roberts, R. L., Jiang, M., Yu, X., Shappell, S. B., Caprioli, R. M., Stoffel, M., Hayward, S. W. and Matusik, R. J. (2005). Forkhead box A1 regulates prostate ductal morphogenesis and promotes epithelial cell maturation. Development 132, 3431-3443.
34) Wang, K., Saito, M., Bisikirska, B. C., Alvarez, M. J., Lim, W. K., Rajbhandari, P., Shen, Q., Nemenman, I., Basso, K., Margolin, A. A., Klein, U., Dalla-Favera, R. and Califano, A. (2009). Genome-wide identification of post-translational modulators of transcription factor activity in human B cells. Nat Biotechnol 27, 829-839.
35) Wang, K., Alvarez, M. J., Bisikirska, B. C., Linding, R., Basso, K., Dalla Favera, R. and Califano, A. (2009). Dissecting the interface between signaling and transcriptional regulation in human B cells. Pac Symp Biocomput 264-275.
36) Lim, W. K., Lyashenko, E. and Califano, A. (2009). Master regulators used as breast cancer metastasis classifier. Pac Symp Biocomput 504-515.
37) Taylor, B. S., Schultz, N., Hieronymus, H., Gopalan, A., Xiao, Y., Carver, B. S., Arora, V. K., Kaushik, P., Cerami, E., Reva, B., Antipin, Y., Mitsiades, N., Landers, T., Dolgalev, I., Major, J. E., Wilson, M., Socci, N. D., Lash, A. E., Heguy, A., Eastham, J. A., Scher, H. I., Reuter, V. E., Scardino, P. T., Sander, C., Sawyers, C. L. and Gerald, W. L. (2010). Integrative genomic profiling of human prostate cancer. Cancer Cell 18, 11-22.
38) Li, R., Liang, J., Ni, S., Zhou, T., Qing, X., Li, H., He, W., Chen, J., Li, F., Zhuang, Q., Qin, B., Xu, J., Li, W., Yang, J., Gan, Y., Qin, D., Feng, S., Song, H., Yang, D., Zhang, B., Zeng, L., Lai, L., Esteban, M. A. and Pei, D. (2010). A mesenchymal-to-epithelial transition initiates and is required for the nuclear reprogramming of mouse fibroblasts. Cell Stem Cell 7, 51-63.
39) Samavarchi-Tehrani, P., Golipour, A., David, L., Sung, H. K., Beyer, T. A., Datti, A., Woltjen, K., Nagy, A. and Wrana, J. L. (2010). Functional genomics reveals a BMP-driven mesenchymal-to-epithelial transition in the initiation of somatic cell reprogramming. Cell Stem Cell 7, 64-77.
40) He, H. H., Meyer, C. A., Shin, H., Bailey, S. T., Wei, G., Wang, Q., Zhang, Y., Xu, K., Ni, M., Lupien, M., Mieczkowski, P., Lieb, J. D., Zhao, K., Brown, M. and Liu, X. S. (2010). Nucleosome dynamics define transcriptional enhancers. Nat Genet 42, 343-347.
41) Berman, D. M., Desai, N., Wang, X., Karhadkar, S. S., Reynon, M., Abate-Shen, C., Beachy, P. A. and Shen, M. M. (2004). Roles for Hedgehog signaling in androgen production and prostate ductal morphogenesis. Dev Biol 267, 387-398.
42) Gao, H., Ouyang, X., Banach-Petrosky, W. A., Gerald, W. L., Shen, M. M. and Abate-Shen, C. (2006). Combinatorial activities of Akt and B-Raf/Erk signaling in a mouse model of androgen-independent prostate cancer. Proc Natl Acad Sci USA 103, 14477-14482.
43) Kim, M. J., Bhatia-Gaur, R., Banach-Petrosky, W. A., Desai, N., Wang, Y., Hayward, S. W., Cunha, G. R., Cardiff, R. D., Shen, M. M. and Abate-Shen, C. (2002). Nkx3.1 mutant mice recapitulate early stages of prostate carcinogenesis. Cancer Res. 62, 2999-3004.
44) Carey, B. W., Markoulaki, S., Beard, C., Hanna, J. and Jaenisch, R. (2010). Single-gene transgenic mouse strains for reprogramming adult somatic cells. Nat Methods 7, 56-59.
45) Shi, X., Gipp, J. and Bushman, W. (2007). Anchorage-independent culture maintains prostate stem cells. Dev Biol 312, 396-406.
46) Lukacs, R. U., Goldstein, A. S., Lawson, D. A., Cheng, D. and Witte, O. N. (2010). Isolation, cultivation and characterization of adult murine prostate stem cells. Nat Protoc 5, 702-713.
47) Subramanian, A., Tamayo, P., Mootha, V. K., Mukherjee, S., Ebert, B. L., Gillette, M. A., Paulovich, A., Pomeroy, S. L., Golub, T. R., Lander, E. S. and Mesirov, J. P. (2005). Gene set enrichment analysis: a knowledge-based approach for interpreting genome-wide expression profiles. Proc Natl Acad Sci USA 102, 15545-15550.
48) Julio, M. K., Alvarez, M. J., Galli, A., Chu, J., Price, S. M., Califano, A. and Shen, M. M. (2011). Regulation of extra-embryonic endoderm stem cell differentiation by Nodal and Cripto signaling. Development 138, 3885-3895.
49) Shen, M. M. and Abate-Shen, C. (2010). Molecular genetics of prostate cancer: new prospects for old challenges. Genes Dev 24, 1967-2000.

### A-REFERENCES CITED

1) Sancho-Martinez, I., Baek, S. H. and Izpisua Belmonte, J. C. (2012). Lineage conversion methodologies meet the reprogramming toolbox. Nat Cell Biol 14, 892-899.
A2) Morris, S. A. and Daley, G. Q. (2013). A blueprint for engineering cell fate: current technologies to reprogram cell identity. Cell Res 23, 33-48.
A3) Davis, R. L., Weintraub, H. and Lassar, A. B. (1987). Expression of a single transfected cDNA converts fibroblasts to myoblasts. Cell 51, 987-1000.
A4) Takahashi, K. and Yamanaka, S. (2006). Induction of pluripotent stem cells from mouse embryonic and adult fibroblast cultures by defined factors. Cell 126, 663-676.
A5) Ieda, M., Fu, J. D., Delgado-Olguin, P., Vedantham, V., Hayashi, Y., Bruneau, B. G. and Srivastava, D. (2010). Direct reprogramming of fibroblasts into functional cardiomyocytes by defined factors. Cell 142, 375-386.
A6) Vierbuchen, T., Ostermeier, A., Pang, Z. P., Kokubu, Y., Sudhof, T. C. and Wernig, M. (2010). Direct conversion of fibroblasts to functional neurons by defined factors. Nature 463, 1035-1041.
A7) Pang, Z. P., Yang, N., Vierbuchen, T., Ostermeier, A., Fuentes, D. R., Yang, T. Q., Citri, A., Sebastiano, V., Marro, S., Sudhof, T. C. and Wernig, M. (2011). Induction of human neuronal cells by defined transcription factors. Nature
A8) Caiazzo, M., Dell'Anno, M. T., Dvoretskova, E., Lazarevic, D., Taverna, S., Leo, D., Sotnikova, T. D., Menegon, A., Roncaglia, P., Colciago, G., Russo, G., Carninci, P., Pezzoli, G., Gainetdinov, R. R., Gustincich, S., Dityatev, A. and Broccoli, V. (2011). Direct generation of functional dopaminergic neurons from mouse and human fibroblasts. Nature 476, 224-227.
A9) Qiang, L., Fujita, R., Yamashita, T., Angulo, S., Rhinn, H., Rhee, D., Doege, C., Chau, L., Aubry, L., Vanti, W. B., Moreno, H. and Abeliovich, A. (2011). Directed conversion of Alzheimer's disease patient skin fibroblasts into functional neurons. Cell 146, 359-371.
A10) Szabo, E., Rampalli, S., Risueno, R. M., Schnerch, A., Mitchell, R., Fiebig-Comyn, A., Levadoux-Martin, M. and Bhatia, M. (2010). Direct conversion of human fibroblasts to multilineage blood progenitors. Nature 468, 521-526.
A11) Efe, J. A., Hilcove, S., Kim, J., Zhou, H., Ouyang, K., Wang, G., Chen, J. and Ding, S. (2011). Conversion of mouse fibroblasts into cardiomyocytes using a direct reprogramming strategy. Nat Cell Biol 13, 215-222.
A12) Kim, J., Efe, J. A., Zhu, S., Talantova, M., Yuan, X., Wang, S., Lipton, S. A., Zhang, K. and Ding, S. (2011). Direct reprogramming of mouse fibroblasts to neural progenitors. Proc Natl Acad Sci USA 108, 7838-7843.
A13) Thier, M., Worsdorfer, P., Lakes, Y. B., Gorris, R., Herms, S., Opitz, T., Seiferling, D., Quandel, T., Hoffmann, P., Nothen, M. M., Brustle, O. and Edenhofer, F. (2012). Direct conversion of fibroblasts into stably expandable neural stem cells. Cell Stem Cell 10, 473-479.
A14) Cunha, G. R. (2008). Mesenchymal-epithelial interactions: past, present, and future. Differentiation 76, 578-586.
A15) Cunha, G. R., Donjacour, A. A., Cooke, P. S., Mee, S., Bigsby, R. M., Higgins, S. J. and Sugimura, Y. (1987). The endocrinology and developmental biology of the prostate. Endocrine Rev. 8, 338-362.
A16) Bhatia-Gaur, R., Donjacour, A. A., Sciavolino, P. J., Kim, M., Desai, N., Young, P., Norton, C. R., Gridley, T., Cardiff, R. D., Cunha, G. R., Abate-Shen, C. and Shen, M. M. (1999). Roles for Nkx3.1 in prostate development and cancer. Genes Dev. 13, 966-977.
A17) Berman, D. M., Desai, N., Wang, X., Karhadkar, S. S., Reynon, M., Abate-Shen, C., Beachy, P. A. and Shen, M. M. (2004). Roles for Hedgehog signaling in androgen production and prostate ductal morphogenesis. Dev Biol 267, 387-398.
A18) Gao, H., Ouyang, X., Banach-Petrosky, W. A., Gerald, W. L., Shen, M. M. and Abate-Shen, C. (2006). Combinatorial activities of Akt and B-Raf/Erk signaling in a mouse model of androgen-independent prostate cancer. Proc Natl Acad Sci USA 103, 14477-14482.
A19) Kim, M. J., Bhatia-Gaur, R., Banach-Petrosky, W. A., Desai, N., Wang, Y., Hayward, S. W., Cunha, G. R., Cardiff, R. D., Shen, M. M. and Abate-Shen, C. (2002). Nkx3.1 mutant mice recapitulate early stages of prostate carcinogenesis. Cancer Res. 62, 2999-3004.
A20) Wang, X., Kruithof-de Julio, M., Economides, K. D., Walker, D., Yu, H., Halili, M. V., Hu, Y.-P., Price, S. M., Abate-Shen, C. and Shen, M. M. (2009). A luminal epithelial stem cell that is a cell of origin for prostate cancer. Nature 461, 495-500.
A21) Wang, Z. A., Mitrofanova, A., Bergren, S. K., Abate-Shen, C., Cardiff, R. D., Califano, A. and Shen, M. M. (2013). Lineage analysis of basal epithelial cells reveals their unexpected plasticity and supports a cell of origin model for prostate cancer heterogeneity. Nat Cell Biol, in press.
A22) Goldstein, A. S., Lawson, D. A., Cheng, D., Sun, W., Garraway, I. P. and Witte, O. N. (2008). Trop2 identifies a subpopulation of murine and human prostate basal cells with stem cell characteristics. Proc Natl Acad Sci USA 105, 20882-20887.
A23) Lawson, D. A., Xin, L., Lukacs, R. U., Cheng, D. and Witte, O. N. (2007). Isolation and functional characterization of murine prostate stem cells. Proc Natl Acad Sci USA 104, 181-186.
A24) Taylor, R. A., Cowin, P. A., Cunha, G. R., Pera, M., Trounson, A. 0., Pedersen, J. and Risbridger, G. P. (2006). Formation of human prostate tissue from embryonic stem cells. Nat Methods 3, 179-181.
A25) Cunha, G. R. (1975). Age-dependent loss of sensitivity of female urogenital sinus to androgenic conditions as a function of the epithelia-stromal interaction in mice. Endocrinology 97, 665-673.
A26) Cunha, G. R., Fujii, H., Neubauer, B. L., Shannon, J. M., Sawyer, L. and Reese, B. A. (1983). Epithelial-mesenchymal interactions in prostatic development. I. Morphological observations of prostatic induction by urogenital sinus mesenchyme in epithelium of the adult rodent urinary bladder. J Cell Biol 96, 1662-1670.
A27) Taylor, R. A., Wang, H., Wilkinson, S. E., Richards, M. G., Britt, K. L., Vaillant, F., Lindeman, G. J., Visvader, J. E., Cunha, G. R., St John, J. and Risbridger, G. P. (2009). Lineage enforcement by inductive mesenchyme on adult epithelial stem cells across developmental germ layers. Stem Cells 27, 3032-3042.
A28) Sneddon, J. B., Borowiak, M. and Melton, D. A. (2012). Self-renewal of embryonic-stem-cell-derived progenitors by organ-matched mesenchyme. Nature 491, 765-768.
A29) Margolin, A. A., Nemenman, I., Basso, K., Wiggins, C., Stolovitzky, G., Dalla Favera, R. and Califano, A. (2006). ARACNE: an algorithm for the reconstruction of gene regulatory networks in a mammalian cellular context. BMC Bioinformatics 7 Suppl 1, S7.
A30) Basso, K., Margolin, A. A., Stolovitzky, G., Klein, U., Dalla-Favera, R. and Califano, A. (2005). Reverse engineering of regulatory networks in human B cells. Nat Genet 37, 382-390.
A31) Wang, K., Saito, M., Bisikirska, B. C., Alvarez, M. J., Lim, W. K., Rajbhandari, P., Shen, Q., Nemenman, I., Basso, K., Margolin, A. A., Klein, U., Dalla-Favera, R. and Califano, A. (2009). Genome-wide identification of post-translational modulators of transcription factor activity in human B cells. Nat Biotechnol 27, 829-839.
A32) Carro, M. S., Lim, W. K., Alvarez, M. J., Bollo, R. J., Zhao, X., Snyder, E. Y., Sulman, E. P., Anne, S. L., Doetsch, F., Colman, H., Lasorella, A., Aldape, K., Califano, A. and Iavarone, A. (2010). The transcriptional network for mesenchymal transformation of brain tumours. Nature 463, 318-325.
A33) Lefebvre, C., Rajbhandari, P., Alvarez, M. J., Bandaru, P., Lim, W. K., Sato, M., Wang, K., Sumazin, P., Kustagi, M., Bisikirska, B. C., Basso, K., Beltrao, P., Krogan, N., Gautier, J., Dalla-Favera, R. and Califano, A. (2010). A human B-cell interactome identifies MYB and FOXM1 as master regulators of proliferation in germinal centers. Mol Syst Biol 6, 377.
A34) Zhao, X., D, D. A., Lim, W. K., Brahmachary, M., Carro, M. S., Ludwig, T., Cardo, C. C., Guillemot, F., Aldape, K., Califano, A., Iavarone, A. and Lasorella, A. (2009). The N-Myc-DLL3 cascade is suppressed by the ubiquitin ligase Huwe1 to inhibit proliferation and promote neurogenesis in the developing brain. Dev Cell 17, 210-221.
A35) Perez-Pinera, P., Ousterout, D. G. and Gersbach, C. A. (2012). Advances in targeted genome editing. Curr Opin Chem Biol 16, 268-277.
A36) Miller, J. C., Tan, S., Qiao, G., Barlow, K. A., Wang, J., Xia, D. F., Meng, X., Paschon, D. E., Leung, E., Hinkley, S. J., Dulay, G. P., Hua, K. L., Ankoudinova, I., Cost, G. J., Urnov, F. D., Zhang, H. S., Holmes, M. C., Zhang, L., Gregory, P. D. and Rebar, E. J. (2011). A TALE nuclease architecture for efficient genome editing. Nat Biotechnol 29, 143-148.
A37) Hockemeyer, D., Wang, H., Kiani, S., Lai, C. S., Gao, Q., Cassady, J. P., Cost, G. J., Zhang, L., Santiago, Y., Miller, J. C., Zeitler, B., Cherone, J. M., Meng, X., Hinkley, S. J., Rebar, E. J., Gregory, P. D., Urnov, F. D. and Jaenisch, R. (2011). Genetic engineering of human pluripotent cells using TALE nucleases. Nat Biotechnol 29, 731-734.
A38) Reyon, D., Tsai, S. Q., Khayter, C., Foden, J. A., Sander, J. D. and Joung, J. K. (2012). FLASH assembly of TALENs for high-throughput genome editing. Nat Biotechnol 30, 460-465.
A39) Goldstein, A. S., Huang, J., Guo, C., Garraway, I. P. and Witte, O. N. (2010). Identification of a cell of origin for human prostate cancer. Science 329, 568-571.
A40) Shen, M. M. and Abate-Shen, C. (2010). Molecular genetics of prostate cancer: new prospects for old challenges. Genes Dev 24, 1967-2000.
A41) Nemajerova, A., Kim, S. Y., Petrenko, O. and Moll, U. M. (2012). Two-factor reprogramming of somatic cells to pluripotent stem cells reveals partial functional redundancy of Sox2 and Klf4. Cell Death Differ 19, 1268-1276.
A42) Li, R., Liang, J., Ni, S., Zhou, T., Qing, X., Li, H., He, W., Chen, J., Li, F., Zhuang, Q., Qin, B., Xu, J., Li, W., Yang, J., Gan, Y., Qin, D., Feng, S., Song, H., Yang, D., Zhang, B., Zeng, L., Lai, L., Esteban, M. A. and Pei, D. (2010). A mesenchymal-to-epithelial transition initiates and is required for the nuclear reprogramming of mouse fibroblasts. Cell Stem Cell 7, 51-63.
A43) Samavarchi-Tehrani, P., Golipour, A., David, L., Sung, H. K., Beyer, T. A., Datti, A., Woltjen, K., Nagy, A. and Wrana, J. L. (2010). Functional genomics reveals a BMP-driven mesenchymal-to-epithelial transition in the initiation of somatic cell reprogramming. Cell Stem Cell 7, 64-77.
A44) Stadtfeld, M., Maherali, N., Borkent, M. and Hochedlinger, K. (2010). A reprogrammable mouse strain from gene-targeted embryonic stem cells. Nat Methods 7, 53-55.
A45) He, H. H., Meyer, C. A., Shin, H., Bailey, S. T., Wei, G., Wang, Q., Zhang, Y., Xu, K., Ni, M., Lupien, M., Mieczkowski, P., Lieb, J. D., Zhao, K., Brown, M. and Liu, X. S. (2010). Nucleosome dynamics define transcriptional enhancers. Nat Genet 42, 343-347.
A46) Marker, P. C., Donjacour, A. A., Dahiya, R. and Cunha, G. R. (2003). Hormonal, cellular, and molecular control of prostatic development. Dev Biol 253, 165-174.
A47) Gao, N., Ishii, K., Mirosevich, J., Kuwajima, S., Oppenheimer, S. R., Roberts, R. L., Jiang, M., Yu, X., Shappell, S. B., Caprioli, R. M., Stoffel, M., Hayward, S. W. and Matusik, R. J. (2005). Forkhead box A1 regulates prostate ductal morphogenesis and promotes epithelial cell maturation. Development 132, 3431-3443.
A48) Wang, Q., Li, W., Zhang, Y., Yuan, X., Xu, K., Yu, J., Chen, Z., Beroukhim, R., Wang, H., Lupien, M., Wu, T., Regan, M. M., Meyer, C. A., Carroll, J. S., Manrai, A. K., Janne, O. A., Balk, S. P., Mehra, R., Han, B., Chinnaiyan, A. M., Rubin, M. A., True, L., Fiorentino, M., Fiore, C., Loda, M., Kantoff, P. W., Liu, X. S. and Brown, M. (2009). Androgen receptor regulates a distinct transcription program in androgen-independent prostate cancer. Cell 138, 245-256.
A49) Sahu, B., Laakso, M., Ovaska, K., Mirtti, T., Lundin, J., Rannikko, A., Sankila, A., Turunen, J. P., Lundin, M., Konsti, J., Vesterinen, T., Nordling, S., Kallioniemi, O., Hautaniemi, S. and Janne, O. A. (2011). Dual role of FoxA1 in androgen receptor binding to chromatin, androgen signalling and prostate cancer. EMBO J 30, 3962-3976.
A50) Lupien, M., Eeckhoute, J., Meyer, C. A., Wang, Q., Zhang, Y., Li, W., Carroll, J. S., Liu, X. S. and Brown, M. (2008). FoxA1 translates epigenetic signatures into enhancer-driven lineage-specific transcription. Cell 132, 958-970.
A51) Kruithof-de Julio, M., Shibata, M., Desai, N., Reynon, M., Halili, M. V., Hu, Y.-P., Price, S. M., Abate-Shen, C. and Shen, M. M. Canonical Wnt signaling regulates Nkx3.1 expression and luminal epithelial differentiation during prostate organogenesis. submitted.
A52) Tan, P. Y., Chang, C. W., Chng, K. R., Wansa, K. D., Sung, W. K. and Cheung, E. (2012). Integration of regulatory networks by NKX3-1 promotes androgen-dependent prostate cancer survival. Mol Cell Biol 32, 399-414.
A53) Xu, J., Watts, J. A., Pope, S. D., Gadue, P., Kamps, M., Plath, K., Zaret, K. S. and Smale, S. T. (2009). Transcriptional competence and the active marking of tissue-specific enhancers by defined transcription factors in embryonic and induced pluripotent stem cells. Genes Dev 23, 2824-2838.
A54) DeGraff, D. J., Clark, P. E., Cates, J. M., Yamashita, H., Robinson, V. L., Yu, X., Smolkin, M. E., Chang, S. S., Cookson, M. S., Herrick, M. K., Shariat, S. F., Steinberg, G. D., Frierson, H. F., Wu, X. R., Theodorescu, D. and Matusik, R. J. (2012). Loss of the urothelial differentiation marker FOXA1 is associated with high grade, late stage bladder cancer and increased tumor proliferation. PLoS One 7, e36669.
A55) Hayward, S. W., Haughney, P. C., Rosen, M. A., Greulich, K. M., Weier, H. U., Dahiya, R. and Cunha, G. R. (1998). Interactions between adult human prostatic epithelium and rat urogenital sinus mesenchyme in a tissue recombination model. Differentiation 63, 131-140.
A56) Margolin, A. A., Wang, K., Lim, W. K., Kustagi, M., Nemenman, I. and Califano, A. (2006). Reverse engineering cellular networks. Nat Protoc 1, 662-671.
A57) Taylor, B. S., Schultz, N., Hieronymus, H., Gopalan, A., Xiao, Y., Carver, B. S., Arora, V. K., Kaushik, P., Cerami, E., Reva, B., Antipin, Y., Mitsiades, N., Landers, T., Dolgalev, I., Major, J. E., Wilson, M., Socci, N. D., Lash, A. E., Heguy, A., Eastham, J. A., Scher, H. I., Reuter, V. E., Scardino, P. T., Sander, C., Sawyers, C. L. and Gerald, W. L. (2010). Integrative genomic profiling of human prostate cancer. Cancer Cell 18, 11-22.
A58) Pritchard, C., Mecham, B., Dumpit, R., Coleman, I., Bhattacharjee, M., Chen, Q., Sikes, R. A. and Nelson, P. S. (2009). Conserved gene expression programs integrate mammalian prostate development and tumorigenesis. Cancer Res 69, 1739-1747.
A59) Hu, Y. and Smyth, G. K. (2009). ELDA: extreme limiting dilution analysis for comparing depleted and enriched populations in stem cell and other assays. J Immunol Methods 347, 70-78.
A60) Kruithof-de Julio, M., Alvarez, M. J., Galli, A., Chu, J., Price, S. M., Califano, A. and Shen, M. M. (2011). Regulation of extra-embryonic endoderm stem cell differentiation by Nodal and Cripto signaling. Development 138, 3885-3895.
A61) Brambrink, T., Foreman, R., Welstead, G. G., Lengner, C. J., Wernig, M., Suh, H. and Jaenisch, R. (2008). Sequential expression of pluripotency markers during direct reprogramming of mouse somatic cells. Cell Stem Cell 2, 151-159.
A62) Nakagawa, M., Koyanagi, M., Tanabe, K., Takahashi, K., Ichisaka, T., Aoi, T., Okita, K., Mochiduki, Y., Takizawa, N. and Yamanaka, S. (2008). Generation of induced pluripotent stem cells without Myc from mouse and human fibroblasts. Nat Biotechnol 26, 101-106.
A63) Wernig, M., Meissner, A., Cassady, J. P. and Jaenisch, R. (2008). c-Myc is dispensable for direct reprogramming of mouse fibroblasts. Cell Stem Cell 2, 10-12.
A64) Lengner, C. J., Camargo, F. D., Hochedlinger, K., Welstead, G. G., Zaidi, S., Gokhale, S., Scholer, H. R., Tomilin, A. and Jaenisch, R. (2007). Oct4 expression is not required for mouse somatic stem cell self-renewal. Cell Stem Cell 1, 403-415.
A65) Ousset, M., Van Keymeulen, A., Bouvencourt, G., Sharma, N., Achouri, Y., Simons, B. D. and Blanpain, C. (2012). Multipotent and unipotent progenitors contribute to prostate postnatal development. Nat Cell Biol 14, 1131-1138.
A66) Prins, G. S. and Putz, O. (2008). Molecular signaling pathways that regulate prostate gland development. Differentiation 76, 641-659.
A67) Ferrer-Vaquer, A., Piliszek, A., Tian, G., Aho, R. J., Dufort, D. and Hadjantonakis, A. K. (2010). A sensitive and bright single-cell resolution live imaging reporter of Wnt/beta-catenin signaling in the mouse. BMC Dev Biol 10, 121.
A68) Shaw, A., Papadopoulos, J., Johnson, C. and Bushman, W. (2006). Isolation and characterization of an immortalized mouse urogenital sinus mesenchyme cell line. Prostate 66, 1347-1358.
A69) Mehta, V., Abler, L. L., Keil, K. P., Schmitz, C. T., Joshi, P. S. and Vezina, C. M. (2011). Atlas of Wnt and R-spondin gene expression in the developing male mouse lower urogenital tract. Dev Dyn 240, 2548-2560.
A70) Simons, B. W., Hurley, P. J., Huang, Z., Ross, A. E., Miller, R., Marchionni, L., Berman, D. M. and Schaeffer, E. M. (2012). Wnt signaling though beta-catenin is required for prostate lineage specification. Dev Biol 371, 246-255.
A71) Francis, J. C., Thomsen, M. K., Taketo, M. M. and Swain, A. (2013). beta-Catenin Is Required for Prostate Development and Cooperates with Pten Loss to Drive Invasive Carcinoma. PLoS Genet 9, e1003180.
A72) Di Cristofano, A., De Acetis, M., Koff, A., Cordon-Cardo, C. and Pandolfi, P. P. (2001). Pten and p27KIP1 cooperate in prostate cancer tumor suppression in the mouse. Nat. Genet. 27, 222-224.
A73) Kim, M. J., Cardiff, R. D., Desai, N., Banach-Petrosky, W. A., Parsons, R., Shen, M. M. and Abate-Shen, C. (2002). Cooperativity of Nkx3.1 and Pten loss of function in a mouse model of prostate carcinogenesis. Proc. Natl. Acad. Sci. USA 99, 2884-2889.
A74) Abate-Shen, C., Banach-Petrosky, W. A., Sun, X., Economides, K. D., Desai, N., Gregg, J. P., Borowsky, A. D., Cardiff, R. D. and Shen, M. M. (2003). Nkx3.1; Pten mutant mice develop invasive prostate adenocarcinoma and lymph node metastases. Cancer Res. 63, 3886-3890.
A75) Wang, S., Gao, J., Lei, Q., Rozengurt, N., Pritchard, C., Jiao, J., Thomas, G. V., Li, G., Roy-Burman, P., Nelson, P. S., Liu, X. and Wu, H. (2003). Prostate-specific deletion of the murine Pten tumor suppressor gene leads to metastatic prostate cancer. Cancer Cell 4, 209-221.
A76) Chen, Z., Trotman, L. C., Shaffer, D., Lin, H. K., Dotan, Z. A., Niki, M., Koutcher, J. A., Scher, H. I., Ludwig, T., Gerald, W., Cordon-Cardo, C. and Pandolfi, P. P. (2005). Crucial role of p53-dependent cellular senescence in suppression of Pten-deficient tumorigenesis. Nature 436, 725-730.
A77) Luo, J., Zha, S., Gage, W. R., Dunn, T. A., Hicks, J. L., Bennett, C. J., Ewing, C. M., Platz, E. A., Ferdinandusse, S., Wanders, R. J., Trent, J. M., Isaacs, W. B. and De Marzo, A. M. (2002). Alpha-methylacyl-CoA racemase: a new molecular marker for prostate cancer. Cancer Res 62, 2220-2226.
A78) Barbieri, C. E., Baca, S. C., Lawrence, M. S., Demichelis, F., Blattner, M., Theurillat, J. P., White, T. A., Stojanov, P., Van Allen, E., Stransky, N., Nickerson, E., Chae, S. S., Boysen, G., Auclair, D., Onofrio, R. C., Park, K., Kitabayashi, N., Macdonald, T. Y., Sheikh, K., Vuong, T., Guiducci, C., Cibulskis, K., Sivachenko, A., Carter, S. L., Saksena, G., Voet, D., Hussain, W. M., Ramos, A. H., Winckler, W., Redman, M. C., Ardlie, K., Tewari, A. K., Mosquera, J. M., Rupp, N., Wild, P. J., Moch, H., Morrissey, C., Nelson, P. S., Kantoff, P. W., Gabriel, S. B., Golub, T. R., Meyerson, M., Lander, E. S., Getz, G., Rubin, M. A. and Garraway, L. A. (2012). Exome sequencing identifies recurrent SPOP, FOXA1 and MED12 mutations in prostate cancer. Nat Genet 44, 685-689.
A79) Vinall, R. L., Chen, J. Q., Hubbard, N. E., Sulaimon, S. S., Shen, M. M., Devere White, R. W. and Borowsky, A. D. (2012). Initiation of prostate cancer in mice by Tp53R270H: evidence for an alternative molecular progression. Dis Model Mech 5, 914-920.
A80) Berger, M. F., Lawrence, M. S., Demichelis, F., Drier, Y., Cibulskis, K., Sivachenko, A. Y., Sboner, A., Esgueva, R., Pflueger, D., Sougnez, C., Onofrio, R., Carter, S. L., Park, K., Habegger, L., Ambrogio, L., Fennell, T., Parkin, M., Saksena, G., Voet, D., Ramos, A. H., Pugh, T. J., Wilkinson, J., Fisher, S., Winckler, W., Mahan, S., Ardlie, K., Baldwin, J., Simons, J. W., Kitabayashi, N., MacDonald, T. Y., Kantoff, P. W., Chin, L., Gabriel, S. B., Gerstein, M. B., Golub, T. R., Meyerson, M., Tewari, A., Lander, E. S., Getz, G., Rubin, M. A. and Garraway, L. A. (2011). The genomic complexity of primary human prostate cancer. Nature 470, 214-220.
A81) Kumar, A., White, T. A., MacKenzie, A. P., Clegg, N., Lee, C., Dumpit, R. F., Coleman, I., Ng, S. B., Salipante, S. J., Rieder, M. J., Nickerson, D. A., Corey, E., Lange, P. H., Morrissey, C., Vessella, R. L., Nelson, P. S. and Shendure, J. (2011). Exome sequencing identifies a spectrum of mutation frequencies in advanced and lethal prostate cancers. Proc Natl Acad Sci USA 108, 17087-17092.
A82) Grasso, C. S., Wu, Y. M., Robinson, D. R., Cao, X., Dhanasekaran, S. M., Khan, A. P., Quist, M. J., Jing, X., Lonigro, R. J., Brenner, J. C., Asangani, I. A., Ateeq, B., Chun, S. Y., Siddiqui, J., Sam, L., Anstett, M., Mehra, R., Prensner, J. R., Palanisamy, N., Ryslik, G. A., Vandin, F., Raphael, B. J., Kunju, L. P., Rhodes, D. R., Pienta, K. J., Chinnaiyan, A. M. and Tomlins, S. A. (2012). The mutational landscape of lethal castration-resistant prostate cancer. Nature 487, 239-243.
A83) Ewing, C. M., Ray, A. M., Lange, E. M., Zuhlke, K. A., Robbins, C. M., Tembe, W. D., Wiley, K. E., Isaacs, S. D., Johng, D., Wang, Y., Bizon, C., Yan, G., Gielzak, M., Partin, A. W., Shanmugam, V., Izatt, T., Sinari, S., Craig, D. W., Zheng, S. L., Walsh, P. C., Montie, J. E., Xu, J., Carpten, J. D., Isaacs, W. B. and Cooney, K. A. (2012). Germline mutations in HOXB13 and prostate-cancer risk. N Engl J Med 366, 141-149.
A84) Choi, N., Zhang, B., Zhang, L., Ittmann, M. and Xin, L. (2012). Adult murine prostate basal and luminal cells are self-sustained lineages that can both serve as targets for prostate cancer initiation. Cancer Cell 21, 253-265.
A85) van der Weyden, L., Shaw-Smith, C. and Bradley, A. (2009). Chromosome engineering in ES cells. Methods Mol Biol 530, 49-77.
A86) Hanna, J., Saha, K., Pando, B., van Zon, J., Lengner, C. J., Creyghton, M. P., van Oudenaarden, A. and Jaenisch, R. (2009). Direct cell reprogramming is a stochastic process amenable to acceleration. Nature 462, 595-601.
A87) Hong, H., Takahashi, K., Ichisaka, T., Aoi, T., Kanagawa, O., Nakagawa, M., Okita, K. and Yamanaka, S. (2009). Suppression of induced pluripotent stem cell generation by the p53-p21 pathway. Nature 460, 1132-1135.
A88) Utikal, J., Polo, J. M., Stadtfeld, M., Maherali, N., Kulalert, W., Walsh, R. M., Khalil, A., Rheinwald, J. G. and Hochedlinger, K. (2009). Immortalization eliminates a roadblock during cellular reprogramming into iPS cells. Nature 460, 1145-1148.
A89) Kawamura, T., Suzuki, J., Wang, Y. V., Menendez, S., Morera, L. B., Raya, A., Wahl, G. M. and Izpisua Belmonte, J. C. (2009) Linking the p53 tumour suppressor pathway to somatic cell reprogramming. Nature 460, 1140-1144.

### B-REFERENCES CITED

B1. Bell, S. M., L. Zhang, A. Mendell, Y. Xu, H. M. Haitchi, J. L. Lessard, and J. A. Whitsett, Kruppel-like factor 5 is required for formation and differentiation of the bladder urothelium. Developmental biology, 2011.
B2. Mfopou, J. K., M. Geeraerts, R. Dejene, S. Van Langenhoven, A. Aberkane, L. A. Van Grunsven, and L. Bouwens, Efficient definitive endoderm induction from mouse embryonic stem cell adherent cultures: a rapid screening model for differentiation studies. Stem Cell Res, 2014. 12(1): p. 166-77.

What is claimed:

1. A method for reprogramming embryonic fibroblast cells in culture to induced epithelial cells, the method comprising:
(a) isolating embryonic fibroblasts (EFs);
(b) transducing EFs with a retrovirus comprising a reprogramming factor;
(c) culturing the transduced EFs for at least 24 hours at about 37° C.; and
(d) culturing the transduced EFs in a serum-free basal epithelial medium to generate induced epithelial cells.

2. The method of claim 1, wherein step (b) results in expression of the reprogramming factor in the EFs.

3. The method of claim 2, wherein the reprogramming factor is transiently expressed.

4. The method of claim 2, wherein the reprogramming factor is constitutively expressed.

5. The method of claim 1, wherein the basal epithelial medium contains EGF, FGF, or a combination thereof.

6. The method of claim 1, wherein (d) is performed about 48 hours after (c).

7. The method of claim 1, wherein the EF has a wild-type genotype, an Oct4-GFP knock-in genotype, or a Nkx3.1-lacZ knock-in genotype.

8. The method of claim 1, wherein the retrovirus is a Rebna retrovirus

9. The method of claim 1, wherein the reprogramming factor is Oct4, Sox2, Klf4, c-Myc, or a combination thereof.

10. The method of claim 1, wherein the induced epithelial cells express cytokeratin 5 (CK5), CK8, CK14, CK18, beta-catenin, E-cadherin, or a combination thereof.

11. The method of claim 1, wherein the induced epithelial cells express EpCAM, CD24, or a combination thereof.

12. The method of claim 1, wherein the induced epithelial cells are stably maintained for at least 3 passages, at least 4 passages, at least 5 passages, at least 6 passages, at least 7 passages, at least 8 passages, at least 9 passages, at least 10 passages, at least 11 passages, at least 12 passages, at least 13 passages, at least 14 passages, or at least 15 passages.

13. The method of claim 1, wherein the induced epithelial cells are further differentiated in prostate epithelia or bladder epithelia.

14. The method of claim 1, wherein the retrovirus is a lentivirus.

15. The method of claim 14, wherein the lentivirus is doxycycline regulated.

16. The method of claim 15, wherein the culturing of (c) is in the presence of doxycycline.

17. The method of claim 16, wherein (d) is performed about 5 to 9 days after (c).

18. An isolated population of induced epithelial cells obtained from the method of claim 1 or 16.

19. The population of induced epithelial cells of claim 18, wherein the cells express cytokeratin 5 (CK5), CK8, CK14, CK18, beta-catenin, E-cadherin, or a combination thereof.

20. A method for reconstituting induced epithelial cells into an organ tissue, the method comprising:
(a) isolating the induced epithelial cells of claim 1 or 16;
(b) transducing the induced epithelial cells with a retrovirus comprising a master regulatory gene;
(c) culturing the transduced epithelial cells;
(d) recombining the transduced epithelial cells with mesenchymal cells; and
(e) performing a graft of the recombined cells of (d) into an immunodeficient subject.

21. The method of claim 20, wherein the transduced epithelial cells are cultured in serum free epithelial media.

22. The method of claim 20, wherein the master regulatory gene is a master regulatory gene for prostate development.

23. The method of claim 22, wherein the master regulatory gene for prostate development comprises NKX3.1, Androgen receptor (AR), FOXA1, FOXA2, or a combination thereof.

24. The method of claim 20, wherein the master regulatory gene is a master regulatory gene for bladder development.

25. The method of claim 24, wherein the master regulatory gene for bladder development comprises KLF5, PPARγ, GRHL3, OVO1, FOXA1, ELF3, EHF, or a combination thereof.

26. The method of claim 20, wherein the graft is maintained in the subject for about 6 to 8 weeks.

27. The method of claim 20, wherein the mesenchymal cells comprise urogenital mesenchyme.

28. The method of claim 20, wherein the mesenchymal cells comprise bladder mesenchyme.

29. The method of claim 20, wherein the graft is a renal graft.

30. The method of claim 20, wherein the organ tissue is prostate epithelial tissue.

31. The method of claim 20, wherein the organ tissue is bladder epithelial tissue.

32. The method of claim 30, wherein the prostate tissue expresses p63, CK5, or a combination thereof, in the basal layer.

33. The method of claim 31, wherein the bladder tissue expresses p63, CK5, or a combination thereof, in the basal layer.

34. The method of claim 30, wherein the prostate tissue expresses AR, CK8, or a combination thereof, in the luminal layer.

35. The method of claim 30, wherein the prostate tissue expresses Probasin, PSA, or a combination thereof.

36. The method of claim 31, wherein the bladder tissue expresses CK8, uroplakins, or a combination thereof.

37. The method of claim 31, wherein the bladder tissue stains positive for the presence of the sub-epithelial connective tissue layer (lamina propria) surrounding the urothelium with Gomori's trichrome.

38. The method of claim 20, wherein the retrovirus is a lentivirus.

39. The method of claim 38, wherein the lentivirus is doxycycline regulated.

40. A method for transdifferentiation of embryonic fibroblast cells into prostate or bladder epithelial tissue, the method comprising:
(a) isolating embryonic fibroblasts (EFs);
(b) transducing EFs with a doxycycline regulated lentivirus comprising Oct4, Sox2, Klf4, c-Myc, or a combination thereof;
(c) culturing the transduced EFs for about 5 to 9 days in serum containing media in the presence of doxycycline;
(d) culturing the transduced EFs in a serum-free basal epithelial medium to generate induced epithelial cells;
(e) transducing the induced epithelial cells with a lentivirus comprising NKX3.1, Androgen receptor (AR), FOXA1, KLF5, or a combination thereof;
(f) recombining the transduced cells of (e) with urogenital or bladder mesenchymal cells, wherein (f) is performed about 5 to 9 days after (e); and
(g) performing a renal graft of the recombined cells of (f) into an immunodeficient subject, wherein (g) is performed about 24 hours after (f).

41. The method of claim 40, wherein the induced epithelial cells express cytokeratin 5 (CK5), CK8, CK14, CK18, beta-catenin, E-cadherin, EpCAM, CD24, or a combination thereof.
